SEQUENCE LISTING (59 sequences)

SEQ ID NO: 1 - protein, 10 aa, synthetic construct
SLYQLENYCN

SEQ ID NO: 2 - protein, 10 aa, synthetic construct
  SITE 3: note = X is dihydroxyphenylalanine
  SITE 9: note = X is Cysteate
SLXQLENYXN

SEQ ID NO: 3 - protein, 10 aa, synthetic construct
  SITE 3: note = X is dihydroxyphenylalanine
  SITE 8: note = X is dihydroxyphenylalanine
  SITE 9: note = X is Cysteate
SLXQLENXXN

SEQ ID NO: 4 - protein, 20 aa, synthetic construct
LVEALYLVCGERGFFYTPKT

SEQ ID NO: 5 - protein, 10 aa, synthetic construct
ERGFFYTPKT

SEQ ID NO: 6 - protein, 10 aa, synthetic construct
ERGYYYTPKT

SEQ ID NO: 7 - protein, 10 aa, synthetic construct
  SITE 6: note = X is dihydroxyphenylalanine
ERGYYXTPKT

SEQ ID NO: 8 - protein, 11 aa, synthetic construct
ERGFFYTPKTR

SEQ ID NO: 9 - protein, 11 aa, synthetic construct
ERGYYYTPKTR

SEQ ID NO: 10 - protein, 10 aa, synthetic construct
YLVCGERGFF

SEQ ID NO: 11 - protein, 12 aa, synthetic construct
LVEALYLVCGER

SEQ ID NO: 12 - protein, 7 aa, synthetic construct
FVNQHLC

SEQ ID NO: 13 - protein, 21 aa, Homo sapiens
GIVEQCCTSICSLYQLENYCN

SEQ ID NO: 14 - protein, 30 aa, Homo sapiens
FVNQHLCGSHLVEALYLVCGERGFFYTPKT

SEQ ID NO: 15 - protein, 10 aa, synthetic construct
  SITE 1: note = N-terminal acetylation
  SITE 3: note = X is dihydroxyphenylalanine
  SITE 9: note = X is Cysteate
SLXQLENYXN

SEQ ID NO: 16 - protein, 10 aa, synthetic construct
  SITE 1: note = N-terminal acetylation
  SITE 3: note = X is dihydroxyphenylalanine
  SITE 8: note = X is dihydroxyphenylalanine
  SITE 9: note = X is Cysteate
SLXQLENXXN

SEQ ID NO: 17 - protein, 10 aa, synthetic construct
DNRDGNVYYF

SEQ ID NO: 18 - protein, 20 aa, synthetic construct
GRKAETELLVYPTCVYLFFG

SEQ ID NO: 19 - protein, 10 aa, synthetic construct
LVEALYLVCG

SEQ ID NO: 20 - protein, 10 aa, synthetic construct
  SITE 1: note = N-terminal acetylation
ERGFFYTPKT

SEQ ID NO: 21 - protein, 11 aa, synthetic construct
  SITE 1: note = N-terminal acetylation
ERGFFYTPKTR

SEQ ID NO: 22 - protein, 9 aa, synthetic construct
AAFVNQHLC

SEQ ID NO: 23 - protein, 9 aa, synthetic construct
  SITE 4: note = X is Cysteate
YLVXGERGF

SEQ ID NO: 24 - protein, 9 aa, synthetic construct
  SITE 3: note = X is Cysteate
LVXGERGFF

SEQ ID NO: 25 - protein, 10 aa, synthetic construct
CGSHLVEALY

SEQ ID NO: 26 - protein, 9 aa, synthetic construct
GFFYTPKTR

SEQ ID NO: 27 - protein, 10 aa, synthetic construct
  SITE 4: note = X is Cysteate
YLVXGERGFF

SEQ ID NO: 28 - protein, 8 aa, synthetic construct
  SITE 7: note = X is Cysteate
YQLENYXN

SEQ ID NO: 29 - protein, 10 aa, synthetic construct
  SITE 5: note = X is Cysteate
LYLVXGERGF

SEQ ID NO: 30 - protein, 10 aa, synthetic construct
RGFFYTPKTR

SEQ ID NO: 31 - protein, 9 aa, synthetic construct
  SITE 8: note = X is Cysteate
LYQLENYXN

SEQ ID NO: 32 - protein, 12 aa, synthetic construct
  SITE 7: note = X is Cysteate
EALYLVXGERGF

SEQ ID NO: 33 - protein, 12 aa, synthetic construct
  SITE 8: note = X is Cysteate
VEALYLVXGERG

SEQ ID NO: 34 - protein, 13 aa, synthetic construct
  SITE 8: note = X is Cysteate
VEALYLVXGERGF

SEQ ID NO: 35 - protein, 11 aa, synthetic construct
  SITE 10: note = X is Cysteate
CSLYQLENYXN

SEQ ID NO: 36 - protein, 13 aa, synthetic construct
  SITE 9: note = X is Cysteate
LVEALYLVXGERG

SEQ ID NO: 37 - protein, 13 aa, synthetic construct
CGERGFFYTPKTR

SEQ ID NO: 38 - protein, 14 aa, synthetic construct
  SITE 9: note = X is Cysteate
LVEALYLVXGERGF

SEQ ID NO: 39 - protein, 15 aa, synthetic construct
  SITE 3: note = X is Cysteate
LVXGERGFFYTPKTR

SEQ ID NO: 40 - protein, 16 aa, synthetic construct
  SITE 4: note = X is Cysteate
YLVXGERGFFYTPKTR

SEQ ID NO: 41 - protein, 10 aa, synthetic construct
RVATVSLPRS

SEQ ID NO: 42 - protein, 12 aa, synthetic construct
KLSSPATLNSRV

SEQ ID NO: 43 - protein, 9 aa, synthetic construct
DSIIAEVKA

SEQ ID NO: 44 - protein, 10 aa, synthetic construct
KSISISVARG

SEQ ID NO: 45 - protein, 9 aa, synthetic construct
GFFYTPKTR

SEQ ID NO: 46 - protein, 10 aa, synthetic construct
RGFFYTPKTR

SEQ ID NO: 47 - protein, 9 aa, synthetic construct
  SITE 8: note = X is Cysteate
LYQLENYXN

SEQ ID NO: 48 - protein, 12 aa, synthetic construct
  SITE 7: note = X is Cysteate
EALYLVXGERGF

SEQ ID NO: 49 - protein, 12 aa, synthetic construct
  SITE 8: note = X is Cysteate
VEALYLVXGERG

SEQ ID NO: 50 - protein, 13 aa, synthetic construct
  SITE 8: note = X is Cysteate
VEALYLVXGERGF

SEQ ID NO: 51 - protein, 11 aa, synthetic construct
  SITE 10: note = X is Cysteate
CSLYQLENYXN

SEQ ID NO: 52 - protein, 13 aa, synthetic construct
  SITE 9: note = X is Cysteate
LVEALYLVXGERG

SEQ ID NO: 53 - protein, 13 aa, synthetic construct
CGERGFFYTPKTR

SEQ ID NO: 54 - protein, 14 aa, synthetic construct
  SITE 9: note = X is Cysteate
LVEALYLVXGERGF

SEQ ID NO: 55 - protein, 9 aa, synthetic construct
  SITE 7: note = H is Oxidised
AAFVNQHLC

SEQ ID NO: 56 - protein, 10 aa, synthetic construct
  SITE 1: note = N-terminal acetylation
SLYQLENYCN

SEQ ID NO: 57 - protein, 10 aa, synthetic construct
  SITE 1: note = N-terminal acetylation
ERGYYYTPKT

SEQ ID NO: 58 - protein, 10 aa, synthetic construct
  SITE 6: note = X is dihydroxyphenylalanine
  SITE 1: note = N-terminal acetylation
ERGYYXTPKT

SEQ ID NO: 59 - protein, 11 aa, synthetic construct
  SITE 1: note = N-terminal acetylation
ERGYYYTPKTR

(19) United States
(12) Patent Application Publication
(10) Pub. No.: US 2026/0072027 A1
(43) Pub. Date: Mar. 12, 2026

(54) Peptides And Method

(71) Applicants: Queen Mary University of London, London (GB); University of Exeter, Exeter (GB)

(72) Inventors: Ahuva Nissim, London (GB); Rocky Strollo, London (GB); Chiara Vinci, London (GB); Paul Winyard, Exeter (GB)

(21) Appl. No.: 19/102,141

(22) PCT Filed: Aug. 10, 2023
(86) PCT No.: PCT/GB2023/052119

§ 371 Date: Feb. 7, 2025

(30) Foreign Application Priority Data
Aug. 10, 2022 (GB) 2211696.6

(51) Int. Cl. G01N 33/564; C07K 14/62; G01N 33/68

(52) U.S. Cl. CPC G01N 33/564; C07K 14/62; G01N 33/6851; G01N 2800/042; G01N 2800/24

(57) ABSTRACT

The invention provides, in some aspects, peptides, kits comprising the same and methods of using such peptides for diagnosing/treating type 1 diabetes (T1D) and latent autoimmune diabetes in adults (LADA) and methods of determining the therapeutic effectiveness of a therapeutic agent in treating T1D/LADA.

### FIELD OF THE INVENTION

The present invention relates to peptide epitopes that are bound by autoantibodies in type 1 diabetes (T1D). The invention specifically relates to the use of such peptides in methods for diagnosing T1D and predicting the response of patients with T1D to drugs. Furthermore, the invention relates to the treatment of patients with T1D.

### BACKGROUND TO THE INVENTION

Type 1 diabetes (T1D) is a condition characterised by a lack of insulin as a result of destruction of insulin producing beta cells in the pancreas. Autoimmunity is believed to be the predominant effector mechanism which leads to the production of a number of autoantibodies that target the beta cells. T1D is primarily associated with children and adults under the age of 40. There is currently no effective therapy available for T1D.

Oxidative stress may be a critical player in the pathogenesis of type 1 diabetes (T1D). Hyperglycemia and high influx of metabolically active immune cells infiltrating the inflamed islets may result in formation of high levels of reactive oxygen species (ROS) within the β-cell microenvironment. Some of the key ROS that are known to be produced are superoxide radical (O₂^{●-}), hydrogen peroxide (H₂O₂), hydroxyl radical (^{●}OH), hypochlorous acid (HOCl), nitric oxide (NO^{●}), peroxynitrite (ONOO⁻), and oxidants derived from glycation as a result of hyperglycemia. High levels of ROS may lead to oxidative posttranslational modification (oxPTM) of β-cell self-proteins and formation of neoepitopes, namely epitopes that were not previously presented to the immune system, and therefore escape immune tolerance and generate autoimmunity. The effect of ROS in inducing autoimmunity toward β-cell specific antigens remains largely unknown. The reason for the breakdown of tolerance to insulin in T1D has so far remained a mystery.

Over 30 years ago, evidence that insulin autoantibodies (IAA) are present in subjects with T1D was published in Science (Palmer et al., Science 222:1337, 1983). IAA are established markers of T1D and may be used for the prediction of this disease. However, they are detected in only half of patients with newly-diagnosed T1D and in fewer patients diagnosed in adult age. Moreover, IAA titres are known to fluctuate during the progression of the disease. There is therefore a need for other biomarkers which can be used to diagnose T1D.

In WO 2016/146979 the present inventors demonstrated that the immune response in type 1 diabetes (T1D) might be directed toward insulin modified by ROS rather than native insulin. Autoantibodies to insulin modified by ROS were shown to be a useful biomarker for T1D prediction, diagnosis and prognosis.

### SUMMARY OF THE INVENTION

Although autoantibodies to insulin modified by ROS were previously shown to be a useful biomarker for T1D prediction, diagnosis and prognosis, the precise epitopes within the oxidised insulin protein to which autoantibodies bind was not known. Determination of the correct epitopes is desirable because it allows for the reliable and consistent production of reagents for detection of autoantibodies. This is particularly important in the case of insulin modified by ROS due to the inherent unpredictability of the ROS modification process. However, this same unpredictability makes epitope determination unusually problematic.

The present inventors have now correctly determined the oxidised peptide sequences that are antigenic in T1D. These oxidised peptides can therefore be used as the basis of an improved method of diagnosis of T1D. Furthermore, the present inventors have determined that the use of multiple oxidised peptides is advantageous, since it allows for a diagnostic method with improved patient coverage.

Accordingly, the present invention provides a peptide comprising an amino acid sequence selected from the group comprising:

(i)
(SEQ ID NO: 1)
SLYQLENYCN
or an oxidised version thereof;
(ii)
(SEQ ID NO: 2)
SL-dihydroxyphenylalanine-QLENY-Cysteate-N;
(iii)
(SEQ ID NO: 3)
SL-dihydroxyphenylalanine-QLEN-
dihydroxyphenylalanine-cysteate-N;
(iv)
(SEQ ID NO: 4)
LVEALYLVCGERGFFYTPKT
or an oxidised version thereof;
(v)
(SEQ ID NO: 5)
ERGFFYTPKT
or an oxidised version thereof;
(vi)
(SEQ ID NO: 6)
ERGYYYTPKT;
(vii)
(SEQ ID NO: 7)
ERGYY-dihydroxyphenylalanine-TPKT;
(viii)
(SEQ ID NO: 8)
ERGFFYTPKTR
or an oxidised version thereof;
(ix)
(SEQ ID NO: 9)
ERGYYYTPKTR;
(x)
(SEQ ID NO: 10)
YLVCGERGFF
or an oxidised version thereof;
(xi)
(SEQ ID NO: 11)
LVEALYLVCGER
or an oxidised version thereof; and
(xii)
(SEQ ID NO: 12)
FVNQHLC
or an oxidised version thereof.

The present invention further provides a method of diagnosing type 1 diabetes (T1D) comprising: testing a sample from a subject for the presence or absence of antibodies against the peptides of the present invention;
wherein the presence of antibodies against the peptides of the present invention in the sample is indicative of T1D in the subject.

### DETAILED DESCRIPTION OF THE INVENTION

In general, the present invention relates to epitopes within the sequence of insulin recognised by autoantibodies in T1D. More specifically, the invention relates to peptides derived from the sequence of insulin to which autoantibodies bind. The present inventors have determined the following peptide sequences to be involved in recognition by autoantibodies in T1D patients:

(a) (Peptide 1)
(SEQ ID NO: 10)
YLVCGERGFF;
(b) (Peptide 2)
(SEQ ID NO: 11)
LVEALYLVCGER;
(c) (Peptide 3)
(SEQ ID NO: 1)
SLYQLENYCN;
(d) (Peptide 4)
(SEQ ID NO: 4)
LVEALYLVCGERGFFYTPKT;
(e) (Peptide 5)
(SEQ ID NO: 12)
FVNQHLC;
or
(f) (Peptide 6)
(SEQ ID NO: 5)
ERGFFYTPKT;
and
(g) (Peptide 6 + R)
(SEQ ID NO: 8)
ERGFFYTPKTR.

Beyond this, the present inventors have identified the exact oxidised forms of these peptides to which auto-antibodies involved in T1D bind. Since a very large number of oxidised peptides derived from ROS-modified insulin is possible, the identification of the specific oxidised peptides involved represents a significant contribution to the art. Surprisingly, it was found that peptides derived from chain A (the alpha chain) of the insulin molecule can also be used. Previously, chain B (the beta chain) of the insulin molecule had been considered more relevant for immunogenicity.

Accordingly, the first aspect the present invention provides a peptide comprising an amino acid sequence selected from the group comprising:

(i)
(SEQ ID NO: 1)
SLYQLENYCN
or an oxidised version thereof;
(ii)
(SEQ ID NO: 2)
SL-dihydroxyphenylalanine-QLENY-Cysteate-N;
(iii)
(SEQ ID NO: 3)
SL-dihydroxyphenylalanine-QLEN-
dihydroxyphenylalanine-cysteate-N;
(iv)
(SEQ ID NO: 4)
LVEALYLVCGERGFFYTPKT
or an oxidised version thereof;
(v)
(SEQ ID NO: 5)
ERGFFYTPKT
or an oxidised version thereof;
(vi)
(SEQ ID NO: 6)
ERGYYYTPKT;
(vii)
(SEQ ID NO: 7)
ERGYY-dihydroxyphenylalanine-TPKT;
(viii)
(SEQ ID NO: 8)
ERGFFYTPKTR
or an oxidised version thereof;
(ix)
(SEQ ID NO: 9)
ERGYYYTPKTR;
(x)
(SEQ ID NO: 10)
YLVCGERGFF
or an oxidised version thereof;
(xi)
(SEQ ID NO: 11)
LVEALYLVCGER
or an oxidised version thereof; and
(xii)
(SEQ ID NO: 12)
FVNQHLC
or an oxidised version thereof.

The peptides of the first aspect of the invention may be referred to herein simply as peptides of the invention. With knowledge of the exact peptide sequences from ROS-modified insulin that bind auto-antibodies in T1D it is possible to produce only the peptides needed for diagnosis. The ability to reliably and consistently produce the precise peptides is advantageous for their use in the methods of the other aspects of the present invention. The peptides may be produced by any suitable method but will typically be synthesised by chemical means. It will be appreciated by the skilled reader that tyrosine is an oxidised form of phenylalanine, dihydroxyphenylalanine (DOPA) is an oxidised form of phenylalanine and that cysteate is an oxidised form of cysteine. In addition, it will be appreciated by the skilled reader that all the above amino acids, including the oxidised forms of amino acids, are typically L-isomers, and that L-cysteate is the conjugate base of L-cysteic acid.

It will be appreciated that the group of peptides of the first aspect may also include the native forms or other oxidised forms of any of peptides 1 to 6 and 6+R identified above, or indeed any other fragments or oxidised fragments of the insulin molecule.

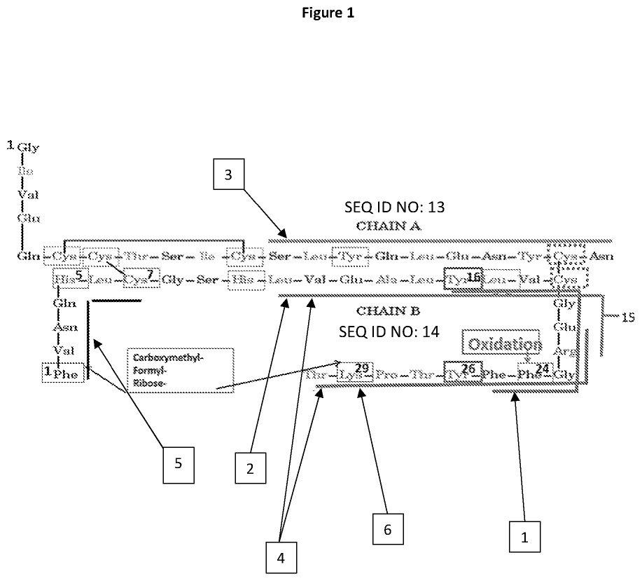

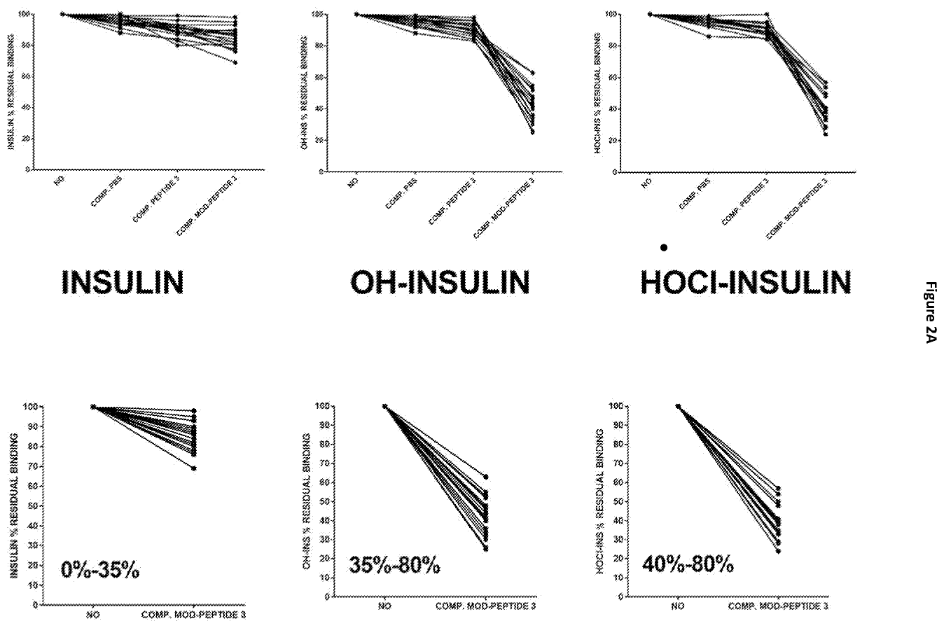

Insulin is a protein dimer of an A-chain and a B-chain (see FIG. 1). FIG. 1 also highlights the peptide identified by the present inventors as involved in binding to auto-antibodies in T1D. The following peptides derive from the B-chain:

(a) (Peptide 1)
(SEQ ID NO: 10)
YLVCGERGFF;
(b) (Peptide 2)
(SEQ ID NO: 11)
LVEALYLVCGER;
(c) (Peptide 4)
(SEQ ID NO: 4)
LVEALYLVCGERGFFYTPKT;
(d) (Peptide 5)
(SEQ ID NO: 12)
FVNQHLC;
(e) (Peptide 6)
(SEQ ID NO: 5)
ERGFFYTPKT;
and
(f) (Peptide 6 + R)
(SEQ ID NO: 8)
ERGFFYTPKTR.

Furthermore, the present inventors have identified the following peptide from the A-chain as involved in auto-antibody binding:

(a) (Peptide 3)
(SEQ ID NO: 1)
SLYQLENYCN.

These peptides may be oxidised in a variety of ways, which makes determination of the precise peptides to which auto-antibodies bind difficult. The present inventors have shown that the following (oxidised) peptides bind to auto-antibodies in T1D:

(i)
(SEQ ID NO: 1)
SLYQLENYCN
or an oxidised version thereof;
(ii)
(SEQ ID NO: 2)
SL-dihydroxyphenylalanine-QLENY-Cysteate-N;
(iii)
(SEQ ID NO: 3)
SL-dihydroxyphenylalanine-QLEN-
dihydroxyphenylalanine-cysteate-N;
(iv)
(SEQ ID NO: 4)
LVEALYLVCGERGFFYTPKT
or an oxidised version thereof;
(v)
(SEQ ID NO: 5)
ERGFFYTPKT
or an oxidised version thereof;
(vi)
(SEQ ID NO: 6)
ERGYYYTPKT;
(vii)
(SEQ ID NO: 7)
ERGYY-dihydroxyphenylalanine-TPKT;
(viii)
(SEQ ID NO: 8)
ERGFFYTPKTR
or an oxidised version thereof;
(ix)
(SEQ ID NO: 9)
ERGYYYTPKTR;
(x)
(SEQ ID NO: 10)
YLVCGERGFF
or an oxidised version thereof;
(xi)
(SEQ ID NO: 11)
LVEALYLVCGER
or an oxidised version thereof; and
(xii)
(SEQ ID NO: 12)
FVNQHLC
or an oxidised version thereof.

As noted previously, tyrosine is an oxidised form of phenylalanine, dihydroxyphenylalanine (DOPA) is an oxidised form of phenylalanine and cysteate is an oxidised form of cysteine.

In a second aspect the present invention relates to a method of diagnosing T1D. In other words, the invention relates to a method of determining whether a subject has T1D or, alternatively, a method of testing for T1D. The method of the invention can also be described as an assay.

The inventors have found that the method of the second aspect of the invention can be used to diagnose patients with new-onset T1D. The method is therefore typically carried out on a sample from a subject who is suspected of having T1D. The subject may, for example, be exhibiting one or more symptoms of T1D such as polydipsia (excessive thirst), polyuria (increased urination), polyphagia (increased appetite), weight loss, fatigue and other symptoms of diabetic ketoacidosis such as nausea, vomiting, abdominal pain, rapid deep sighing and progressive obtundation and loss of consciousness. The method is typically carried out on a sample from a subject who has not previously been treated with insulin or any other therapeutic agent for T1D, for example immunotherapeutics administered as vaccine (e.g. oral insulin, inhaled insulin, etc.) or immunosuppressive drugs.

The method of the second aspect of the invention involves testing a sample from a subject for the presence or absence of antibodies against peptides of the first aspect of the invention. “Peptides of the invention” as used herein refers to peptides of the first aspect of the invention. These peptides are derived from insulin that have been shown to be reactive with auto-antibodies involved in T1D.

The methods of the second and other aspects of the present invention may involve the use of one, two, three, four, five, six, seven, eight, nine, ten, eleven or twelve of the peptides of the first aspect. For example, the method may use any one of the peptide of SEQ ID NOs: 1, 2, 3, 4, 5, 6, 7, 8, 9, 10, 11 or 12. Preferably the method uses two of the peptides, for example the peptides of SEQ ID NOs: 1 and 2; 1 and 3; 1 and 4; 2 and 3; 2 and 4; or 3 and 4. More preferably the method uses three of the peptides, for example the peptides of SEQ ID NOs: 1, 2 and 3; 1, 2 and 4; 1, 3 and 4; or 2, 3 and 4. Most preferably the methods involve the use of four or more, for example one or more peptides of SEQ ID NOs: 1, 2, and 3, the peptide of SEQ ID NO: 4, one or more peptides of SEQ ID NOs: 5, 6 and 7, one or more peptides of SEQ ID NOs: 8 and 9 and optionally the peptides of SEQ ID NOs: 10, 11 and 12. In some instances the methods use all peptides of the first aspect of the invention, for example the peptides of SEQ ID NOs: 1, 2, 3, 4, 5, 6, 7, 8, 9, 10, 11 and 12 in combination.

The method of the second aspect involves testing a sample from a subject for the presence or absence of antibodies against the peptides of the first aspect of the invention. The antibodies to be tested for are auto-antibodies against the peptides of the invention that are present in the subject. As such the antibodies are typically of the immunoglobulin G (IgG) isotype or alternatively of the IgM or IgA isotype. In an embodiment the antibody is IgG. In another embodiment the antibody is IgA.

The sample can be any sample from a subject that could contain auto-antibodies against the peptides of the invention. Typically, the sample is a sample of blood, serum or plasma. In a preferred embodiment, the sample is a blood or serum sample.

The peptides may also find utility as T cell epitopes. In particular, the peptides of the invention may find utility in the detection of T cells which are specific for oxidised forms of insulin. Detection of T cells that are specific for oxidised forms of insulin could be useful in the diagnosis of type 1 diabetes, type 2 diabetes or latent autoimmune diabetes of adulthood.

Accordingly, a first alternative to the second aspect of the invention relates to a method of diagnosing type 1 diabetes (T1D) in a subject comprising: testing a sample from the subject for the presence or absence of T cells that are specific for one or more peptides in accordance with the first aspect of the invention; wherein the presence of T cells that are specific for one or more peptides in accordance with the first aspect of the invention in the sample is indicative of T1D in the subject.

The first alternative to the second aspect of the invention also extends to a method of treating type 1 diabetes (T1D) in a subject in need thereof, comprising:
testing a sample from the subject for the presence or absence of T cells that are specific for peptides of the invention;
identifying the presence of T cells that are specific for peptides of the invention in the sample; and
administering a therapeutic agent for T1D to the subject.

The method according to the first alternative to the second aspect of the invention can be used instead of, or in combination with, the second aspect of the invention.

For example, this can be done in the method of the second aspect of the invention in combination with testing a sample from the subject for the presence or absence of antibodies against one or more peptides of the invention. The invention therefore also encompasses a method of diagnosing type 1 diabetes (T1D) in a subject comprising:
testing a sample from the subject for the presence or absence of antibodies against one or more peptides of the invention and for presence or absence of T cells that are specific for one or more of the peptides of the invention;
wherein the presence of antibodies against one or more peptides of the invention and the presence of said T cells in the sample is indicative of T1D in the subject.

The presence or absence of antibodies against the peptides of the invention can be determined by any suitable method or assay. There is a wide range of different types of immunoassays available that can be used to measure autoantibodies in a sample. Typically, the presence or absence of such antibodies is determined using an assay based on antibody-antigen binding such as an ELISA assay or Western Blotting. In a typical ELISA assay, antigens are attached to a surface. Thus, in the present invention, one or more of the peptides of the invention are attached to a surface. A specific antibody (the primary antibody) is then applied over the surface and binds to the antigen. In this invention, the antibody is in the sample taken from the patient, so the sample is applied over the surface in this step of the method. A second antibody (the secondary antibody) is added, which binds to the primary antibody. The secondary antibody is linked to an enzyme, and then a substance containing the substrate of the enzyme is added. The subsequent reaction produces a detectable signal, typically a colour change in the substrate. The strength of the signal is indicative of the amount of the primary antibody. When the detectable signal is a colour change, a spectrophotometer is often used to give quantitative values for colour strength. ELISA-based assays which employ other types of antibody labels and signal detection methods are available, e.g. labels base on ruthenium complexes, used in conjunction with an electrochemiluminescence-based signal.

In one embodiment, an assay in accordance with the invention comprises: contacting a sample from a subject with one or more peptides of the invention; adding a labelled antibody that binds to anti-modified insulin peptide antibodies to form a complex; and detecting the formation of a complex between anti-modified insulin peptide antibodies and labelled antibody; wherein the detection of said complex is indicative of T1D in the subject.

An antibody-antigen binding assay such as an ELISA assay for use in the present invention is carried out using one or more peptides of the invention as a target for the antibodies in the sample taken from the subject. In such an assay, the peptides of the invention can be prepared by any suitable method. In a suitable ELISA assay, ELISA plates are coated with one or more peptides of the invention as bait to bind auto-antibodies from samples, for example blood or serum samples. Samples are then added to the ELISA plates, optionally with buffer added. Optionally, a non-reacting protein can then be added to block any surface of the ELISA plate that remains uncoated with peptides of the invention. An enzyme such as horseradish peroxidase can then be added. The enzyme is typically conjugated to an anti-human for example IgG but could be IgA or IgM antibody for binding to the antibodies from the sample. Finally, a substrate, for example a chromogenic substrate such as 3,3′,5,5′-tetramethylbenzidine, is added. Activity can then be determined using a suitable method, for example by measuring the optical density (OD). Alternatively, fluorogenic or electrochemiluminescent reporters can be used as appropriate. Suitable ELISA assays for the peptides of the invention are described in the Example herein.

Other suitable assays for peptides of the invention include: the multiplex assays available from Meso Scale Discovery which are based on the MULTI-ARRAY® technology and allow the assaying of many different antigens (for example multiple peptides of the present invention) at the same time; multiplexed bead-based flow cytometry, for example the BD™ Cytometric Bead Array (CBA); and surface plasmon resonance (SPR) based systems, available for example from Biacore. Additional assays include: radiobinding assays (RBA), electrochemiluminescence assays (ECL), and luciferase immuno precipitation system (LIPS) assays.

In some embodiments of the invention, the sample from the subject is compared to a patient control or a sample control. The control sample is typically a sample taken from a subject who is known not to be suffering from T1D, for example a healthy control subject. However, the control sample can also be, for example, from an individual presenting with symptoms of T1D but with no clinical evidence of T1D. The control sample can be from a patient with type 2 diabetes.

In further embodiments, reference positive and/or negative control samples are used. In other embodiments, native insulin (i.e. insulin that has not been modified) may be used as a control. In other embodiments, native human serum albumin (HSA) or bovine serum albumin (BSA) or ROS modified BSA or HSA are used as control antigens. In another embodiment the control antigen is hen egg lysozyme (HEL).

In the methods of the present the invention described herein, the presence or absence of antibodies against peptides of the invention can be determined by comparison to a control or a control sample, typically a control sample that is known not to contain antibodies against peptides of the invention. If the sample from the subject contains significantly higher levels of antibodies against peptides of the invention than in the control sample, this confirms the presence of antibodies against peptides of the invention in the subject. Conversely, if the sample from the subject does not contain significantly higher levels of antibodies against peptides of the invention than in the control sample, this confirms the absence of antibodies against peptides of the invention in the subject. The presence of antibodies against peptides of the invention can be determined, for example, by finding a significant difference between the amounts of antibody in the sample versus a control sample. Statistical tests known in the art can be used, for example the Wilcoxon signed rank sum test or the Mann-Whitney test.

The subject is typically a human subject. However, the methods of the invention also find use in the field of veterinary medicine and can therefore be used to diagnose diabetes in animal subjects, typically mammalian subjects, for example companion animals such as cats and dogs, agricultural animals such as horses, cows, pigs and sheep, or other mammals such as mice, rats, rabbits or monkeys.

The methods of the invention are typically carried out on a sample that has previously been obtained from a subject. Thus, the taking of the sample does not typically form part of the methods of the invention and the methods of the invention are carried out on a sample that has been obtained from a subject. In some embodiments of the invention, however, the method also comprises taking the sample from the subject, for example by taking a blood sample (and optionally then preparing a serum sample from the blood sample).

The main autoantibodies associated with T1D include insulin autoantibodies (IAA), islet cell antibodies (ICA), glutamic acid decarboxylase autoantibodies (GADA), and insulinoma-associated-2 autoantibodies (IA-2A). IAA have become important diagnostic and prognostic tools in T1D, but the diagnostic sensitivity of IAA is not high (˜50%). ICA have been detected in approximately 70% new-onset T1D Caucasian patients. Following diagnosis, ICA frequency decreases, and fewer than 10% of patients still express ICA after 10 years. Approximately 60% onset cases of T1D express GADA. IA-2A and IA-2BA are observed in 60% or more of new-onset type 1 diabetes cases. Zinc transporter-8 (ZnT8) was recently identified as a novel autoantigen in T1D. Autoantibodies to ZnT8 (ZnT8a) have been detected in up to 80% of new-onset T1D.

The inventors have shown that auto-reactivity to insulin is in part due to neo-antigens resulting from oxidative post-translational modification (oxPTM) of insulin by the oxidants present in the β-cell microenvironment. The identity of peptide forms of these neo-antigens has now been confirmed. These peptides form the basis for the present invention.

The present invention therefore provides a new method for improved diagnosis of T1D because it can be used to diagnose patients who test negative for IAA, ICA, GADA, IA2A or ZnT8A.

Accordingly, in one embodiment, the invention provides a method of diagnosing T1D wherein a subject does not express detectable levels of IAA, ICA, GADA, IA2A and/or ZnT8A.

Tests to determine the presence or absence of autoantibodies are known in the art. These include radiobinding assays (RBA), radioimmunoassays (RIA), electrochemiluminescence assays (ECL-IAA), Simoa-based assays and ELISAs.

The method of the second aspect of the invention gives a diagnosis of T1D in a subject. The method can therefore also be used in combination with a method of treating T1D.

Accordingly, the second aspect of the invention also extends to a method of treating type 1 diabetes (T1D) in a subject in need thereof, comprising:
testing a sample from the subject for the presence or absence of antibodies against peptides of the invention;
identifying the presence of antibodies against peptides of the invention in the sample; and administering a therapeutic agent for T1D to the subject.

The therapeutic agent is typically insulin, an immunotherapeutic administered as vaccine (e.g. oral insulin, inhaled insulin, etc.) or an immunosuppressive drug. Most typically, the therapeutic agent is insulin.

This embodiment of the invention also extends to a therapeutic agent for type 1 diabetes (T1D) for use in a method of treating T1D in a subject in need thereof, wherein the method comprises:
testing a sample from the subject for the presence or absence of antibodies against peptides of the invention;
identifying the presence of antibodies against peptides of the invention in the sample; and administering a therapeutic agent for T1D to the subject.

This embodiment of the invention also extends to use of a therapeutic agent for type 1 diabetes (T1D) in the manufacture of a medicament for the treatment of T1D in a subject in need thereof by a method comprising:
testing a sample from the subject for the presence or absence of antibodies against peptides of the invention;
identifying the presence of antibodies against peptides of the invention in the sample; and administering a therapeutic agent for T1D to the subject.

Type 2 diabetes (T2D) is a metabolic disorder usually diagnosed in adults wherein the body produces insufficient amounts of insulin or develops insulin resistance resulting in elevated blood glucose levels. There is no cure for T2D but the condition can be managed through diet, exercise and medication. There is usually no need to take exogenous insulin.

Examples of T2D medication include biguanides (such as metformin), enzyme inhibitors (such as Dipeptidyl peptidase-4 (DPP-4) inhibitors and alpha-glucosidase inhibitors), Sulfonylureas (such as glyburide, glipizide, glimepiride, tolbutamide, chlorpropamide, acetohaxamide and tolazamide), meglitinides (such as repaglinide), thiazolidinediones (such as troglitazone, pioglitazone and rosiglitazone), glucagon-like peptide 1 receptor agonists (such as exenatide, liraglutide, dulaglutide), sodium glucose co-transporter 2 (SGLT2) inhibitors (such as dapagliflozin, empagliflozin, canagliflozin, ertugliflozin) and insulin and insulin analogues (such as lispro).

Although autoimmunity is not typically associated with T2D, up to 12% of people initially diagnosed with T2D have elevated levels of autoantibodies, most commonly glutamic acid decarboxylase antibodies (GADA) (Buzzetti et al., Nature Reviews Endocrinology volume 13, pages 674-686 (2017)). These people, initially diagnosed as having T2D, become unresponsive to diabetes medication and, as in the case of T1D patients, require insulin to control diabetes. This form of diabetes is known as latent autoimmune diabetes of adulthood (LADA) or type 1.5 diabetes because the condition starts off as type 2 diabetes before becoming type 1 diabetes. The presence of autoantibodies is a pre-requisite for diagnosis of LADA and distinguishes this condition from T2D patients who have become insulin-dependent for other reasons.

In a third aspect, the present invention provides a method of diagnosing latent autoimmune diabetes in adults (LADA) in a subject comprising:
testing a sample from the subject for the presence or absence of antibodies against peptides of the invention,
wherein the presence of antibodies against peptides of the invention in the sample is indicative of LADA in the subject.

The method of the third aspect of the invention is typically carried out on a sample from a subject who is known to have T2D. Accordingly, in one embodiment of the third aspect of the invention, the subject has T2D or has been diagnosed with T2D. In another embodiment, the subject is suspected of having T2D, for example because the subject exhibits one or more symptoms of T2D such as polydipsia (excessive thirst), polyuria (increased urination), polyphagia (increased appetite), weight loss and fatigue.

In an embodiment of the third aspect, the sample is a sample of blood or serum or plasma.

The method of the third aspect of the invention gives a diagnosis of LADA in a subject. The method can therefore also be used in combination with a method of treating LADA.

Accordingly, the third aspect of the invention also extends to a method of treating latent autoimmune diabetes in adults (LADA) in a subject in need thereof, comprising:
testing a sample from the subject for the presence or absence of antibodies against peptides of the invention;
   identifying the presence of antibodies against peptides of the invention in the sample; and
   administering a therapeutic agent for LADA to the subject.

The therapeutic agent is typically insulin, an immunotherapeutic administered as vaccine (e.g. oral insulin, inhaled insulin, etc.) or an immunosuppressive drug. Most typically, the therapeutic agent is insulin.

This embodiment of the invention also extends to a therapeutic agent for latent autoimmune diabetes in adults (LADA) for use in a method of treating LADA in a subject in need thereof, wherein the method comprises:
testing a sample from the subject for the presence or absence of antibodies against peptides of the invention;
identifying the presence of antibodies against peptides of the invention in the sample; and
   administering a therapeutic agent for LADA to the subject.

This embodiment of the invention also extends to use of a therapeutic agent for latent autoimmune diabetes in adults (LADA) in the manufacture of a medicament for the treatment of LADA in a subject in need thereof by a method comprising:
testing a sample from the subject for the presence or absence of antibodies against peptides of the invention;
identifying the presence of antibodies against peptides of the invention in the sample; and
   administering a therapeutic agent for LADA to the subject.

In a first alternative to the third aspect of the invention, the present invention provides a method of diagnosing latent autoimmune diabetes in adults (LADA) in a subject comprising:
testing a sample from the subject for the presence or absence of T cells that are specific for peptides of the invention;
wherein the presence of T cells that are specific for peptides of the invention in the sample is indicative of LADA in the subject.

The first alternative to the third aspect of the invention also extends to a method of treating latent autoimmune diabetes in adults (LADA) in a subject in need thereof, comprising:
testing a sample from the subject for the presence or absence of T cells that are specific for peptides of the invention;
   identifying the presence of T cells that are specific for peptides of the invention in the sample; and
   administering a therapeutic agent for LADA to the subject.

The first alternative to the third aspect of the invention also extends to use of a therapeutic agent for latent autoimmune diabetes in adults (LADA) in the manufacture of a medicament for the treatment of LADA in a subject in need thereof by a method comprising:
testing a sample from the subject for the presence or absence of T cells that are specific for peptides of the invention;
   identifying the presence of T cells that are specific for peptides of the invention in the sample; and
   administering a therapeutic agent for LADA to the subject.

The method according to the first alternative to the third aspect of the invention can be used instead of, or in combination with, the third aspect of the invention.

In a fourth aspect, the invention provides a method of determining the therapeutic efficacy of a therapeutic agent to treat a disease associated with oxPTM insulin.

In an embodiment of the fourth aspect, the disease is T1D. In another embodiment the disease is LADA.

According to the fourth aspect, the therapeutic effectiveness of a therapeutic agent to treat T1D or LADA is determined by:
i) determining the level of antibodies against peptides of the invention in a first sample that has been obtained from a subject prior to administering the therapeutic agent to the subject;
ii) determining the level of antibodies against peptides of the invention in a second sample that has been obtained from the subject after administering the therapeutic agent to the subject; and
iii) comparing the levels of antibodies against peptides of the invention between the first and second samples;
wherein a decrease in the level of antibodies against peptides of the invention in the second sample when compared to the first sample is indicative of therapeutic effectiveness of the therapeutic agent.

In some embodiments, the method also includes the steps of obtaining a first and a second sample from the subject and administering the therapeutic agent to the subject. Accordingly, in one embodiment of the third aspect of the invention, the therapeutic effectiveness of a therapeutic agent to treat T1D or LADA is determined by:
i) obtaining a first sample from a subject;
ii) determining the level of antibodies against peptides of the invention in the first sample;
iii) administering the therapeutic agent to the subject;
iv) obtaining a second sample from the subject;
v) determining the level of antibodies against peptides of the invention in the second sample; and
vi) comparing the levels of antibodies against peptides of the invention between the first and second samples;
   wherein a decrease in the level of antibodies against peptides of the invention in the second sample when compared to the first sample is indicative of therapeutic effectiveness of the therapeutic agent.

As used herein, the term “therapeutic agent” refers to a compound that provides a desired biological or pharmacological effect when administered to a subject. Examples of therapeutic agents to treat T1D or LADA include but are not limited to insulin, immunotherapeutics administered as vaccine (e.g. oral insulin, inhaled insulin, etc.) or immunosuppressive drugs.

The method of the fourth aspect of the invention is for identifying whether a subject responds to such a diabetes medication (i.e. a therapeutic agent to treat T1D or LADA). By “respond to a diabetes medication” is meant respond to treatment with a diabetes medication, in other words reduce the severity of the symptoms of T1D in the patient and improve glucose control as measured by blood glucose, HbA1c and C-peptide. Response to treatment with a diabetes medication can be determined by assessing blood glucose control before and after treatment with the diabetes medication.

The method of the fourth aspect of the invention is typically carried out on a sample from a subject that has previously been treated or is currently being treated with one or more diabetes medications. In this aspect, the present invention relates to a method of monitoring whether a patient is responding to therapy with one or more diabetes medications (i.e. a therapeutic agent to treat T1D or LADA).

The method of the fourth aspect of the invention gives an indication of whether a patient responds to a therapeutic agent to treat T1D or LADA. The fourth aspect of the invention therefore also encompasses a method which comprises a further step of treating a patient with a therapeutic agent to treat T1D or LADA, i.e. administering a therapeutic agent for T1D or LADA to the subject. The therapeutic agent is typically insulin, an immunotherapeutic administered as vaccine or an immunosuppressive drug.

In all aspects of the invention, the method of treatment can be of a human or animal subject and this extends equally to uses in both human and veterinary medicine. The therapeutic agent to treat T1D or LADA is preferably administered to a subject in a “therapeutically effective amount”, this being sufficient to show benefit to the subject and/or to ameliorate, eliminate or prevent one or more symptoms of T1D or LADA. As used herein, “treatment” includes any regime that can benefit a human or a non-human animal, preferably a mammal. The treatment may be in respect of an existing condition or may be prophylactic (preventative treatment). The treatment is typically administered to a subject or patient “in need thereof”, i.e. a subject suffering from T1D or LADA.

In this embodiment, the therapeutic agent to treat T1D or LADA can be administered to the subject by any appropriate route, for example by oral (including buccal and sublingual), nasal, topical (including transdermal) or parenteral (including subcutaneous, intramuscular, intravenous, intraperitoneal and intradermal) administration, although when the therapeutic agent to treat T1D or LADA is insulin it will typically be administered to the subject by subcutaneous administration. The therapeutic agent to treat T1D or LADA can be formulated using methods known in the art of pharmacy, for example by admixing the therapeutic agent with carrier(s) or excipient(s) under sterile conditions to form a pharmaceutical composition. Accordingly, in one embodiment the subject is administered a pharmaceutical composition comprising a therapeutic agent to treat T1D or LADA and one or more carriers and/or excipients.

The therapeutic agent to treat T1D or LADA can also be administered in combination with one or more other therapeutically active agents. Accordingly, the pharmaceutical composition for use in accordance with this embodiment of the invention may also comprise one or more other therapeutically active agents in addition to the therapeutic agent to treat T1D or LADA.

Dosages of the therapeutic agent to treat T1D or LADA and/or pharmaceutical composition for use in the present invention can vary between wide limits, depending for example on the particular therapeutic agent used, the age and disease stage of the patient, and a physician will ultimately determine appropriate dosages to be used.

The dosage can be repeated as often as appropriate. If side effects develop, the amount and/or frequency of the dosage can be reduced, in accordance with normal clinical practice.

According to a first alternative of the fourth aspect of the invention, the therapeutic effectiveness of a therapeutic agent to treat T1D or LADA is determined by:
i) determining the level of T cells that are specific for peptides of the invention in a first sample that has been obtained from a subject prior to administering the therapeutic agent to the subject;
ii) determining the level of T cells that are specific for peptides of the invention in a second sample that has been obtained from the subject after administering the therapeutic agent to the subject; and
iii) comparing the levels of T cells that are specific for peptides of the invention between the first and second samples;
wherein a decrease in the level of T cells that are specific for peptides of the invention in the second sample when compared to the first sample is indicative of therapeutic effectiveness of the therapeutic agent.

The level of T cells that are specific for peptides of the present invention can be determined by any appropriate method known in the art (for instance, see Examples).

The method according to the first alternative to the fourth aspect of the invention can be used instead of, or in combination with, the fourth aspect of the invention.

In relation the first alternatives to the second/third/fourth aspects of the invention, T cells that are specific for peptides of the invention can instead, or in addition, be T cells that are reactive to peptides of the invention.

As used herein, T cells that are “specific for” peptides of the invention means those which selectively recognise/bind to an epitope of the peptides of the present invention. T cell specificity can be determined by any appropriate method known in the art.

In relation the first alternatives to the second/third/fourth aspects of the invention, T cells that are specific for peptides of the invention can instead, or in addition, be T cells that are stimulated by peptides of the present invention.

In relation the first alternatives to the second/third/fourth aspects of the invention, the presence or absence of T cells that are specific for/reactive to/stimulated by peptides of the invention can be determined by comparison to a control or a control sample, typically a control sample that is known not to contain T cells that are specific for/reactive to/stimulated by peptides of the invention. If the sample from the subject contains significantly higher levels of T cells that are peptides of the invention than in the control sample, this confirms the presence of T cells that are specific peptides of the invention in the subject. Conversely, if the sample from the subject does not contain significantly higher levels of T cells that are specific for peptides of the invention than in the control sample, this confirms the absence of T cells that are specific for peptides of the invention in the subject. The presence of T cells that are specific for peptides of the invention can be determined, for example, by finding a significant difference between the amounts of T cell in the sample versus a control sample. Statistical tests known in the art can be used, for example the Wilcoxon signed rank sum test or the Mann-Whitney test.

In a fifth aspect the invention provides a kit for diagnosing T1D or LADA in a subject. The kit comprises one or more peptides of the invention and optionally one or more reagents for determining the presence of antibodies against peptides of the invention. The kit is packaged and labelled for example, in a box or container which includes the necessary elements of the kit, and directions and instructions on the use of such diagnostic kit.

In some instances the kits of the present invention may be useful in predicting a subject's risk of developing T1D or LADA.

It will be clear that while the invention is described by reference to various aspects and embodiments, the features of all said aspects and embodiments may be combined with the features of the other aspects and embodiments mutatis mutandis without extending beyond the scope of the present invention.

The present invention will now be further described by way of reference to the following Examples which are present for the purposes of illustration only. In the Examples, reference is made to a number of Figures in which:

FIG. 1. Insulin sequence with peptides marked. Insulin is composed of chain A (SEQ ID NO: 13) and chain B (SEQ ID NO: 14). The locations of the following peptides are marked with solid bars and numbered arrows:

(a) (Peptide 1)
(SEQ ID NO: 10)
YLVCGERGFF;
(b) (Peptide 2)
(SEQ ID NO: 11)
LVEALYLVCGER;
(c) (Peptide 3)
(SEQ ID NO: 1)
SLYQLENYCN;
(d) (Peptide 4)
(SEQ ID NO: 4)
LVEALYLVCGERGFFYTPKT;
(e) (Peptide 5)
(SEQ ID NO: 12)
FVNQHLC;
and
(f) (Peptide 6)
(SEQ ID NO: 5)
ERGFFYTPKT.

Note that peptide 4 is a combination of peptides 2 and 6. In addition, the following oxidised amino acids are marked with boxes:
i. Chain A: Cys6, Cys7, Cys11, Tyr14, Cy20;
ii. Chain B: His5, Cys7, His10, Tyr16, Leu17, Cys19, Phe24, Phe25, Tyr26.

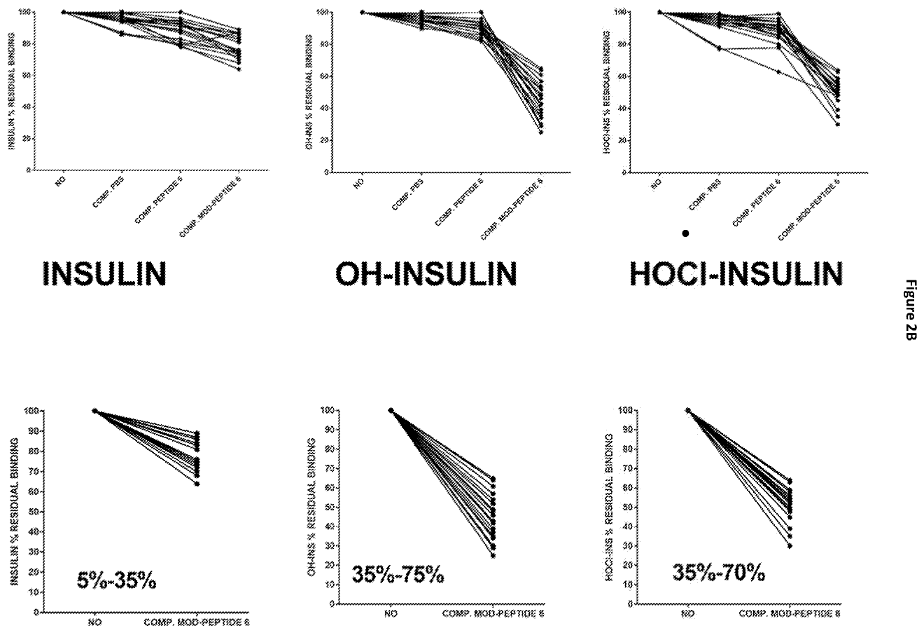

FIG. 2. Competitive ELISA for in house oxidised peptide 3 and 6 and modified forms
A. A competitive displacement assay was performed to confirm binding of autoantibodies to oxidised peptide 3. The binding to the peptide was done by indirect ELISA using native or modified peptide 3. Serum samples from children with type 1 diabetes were pre-incubated with the native synthetic peptide 3 or oxidised peptide 3 before adding to plate coated with either native or oxidised insulin for additional incubation. Bound autoantibodies was probed by anti-human IgG HRP conjugated. No is binding of serum samples without peptide 3 competitor, comp.PBS is preincubation with control PBS, comp.peptide 3 is preincubation with native peptide 3 and comp.mod-peptide 3 is preincubation with oxidised peptide 3. INSULIN is plate coated with native insulin, ^{●}OH-INSULIN is plate coated with insulin oxidised by ^{●}OH and HOCl-INSULIN is plate coated with insulin oxidised by HOCl. Oxidised peptide 3 reduced binding to oxidised whole insulin in the range of 35-80%.
B. A competitive displacement assay was performed to confirm binding of autoantibodies to oxidised peptide 6. The binding to the peptide was done by indirect ELISA using native or modified peptide 6. Serum samples from children with type 1 diabetes were pre-incubated with the native synthetic peptide 6 or oxidised peptide 6 before adding to plate coated with either native or oxidised insulin. Bound autoantibodies was probed by anti-human IgG HRP conjugated. No is binding of serum samples without peptide 6 competitor, comp.PBS is preincubation with control PBS, comp.peptide 6 is preincubation with native peptide 6 and comp.mod-peptide 6 is preincubation with oxidised peptide 6. INSULIN is plate coated with native insulin, ^{●}OH-INSULIN is plate coated with insulin oxidised by ^{●}OH and HOCl-INSULIN is plate coated with insulin oxidised by HOCl. Oxidised peptide 6 reduced binding to oxidised whole insulin in the range of 35-75%.

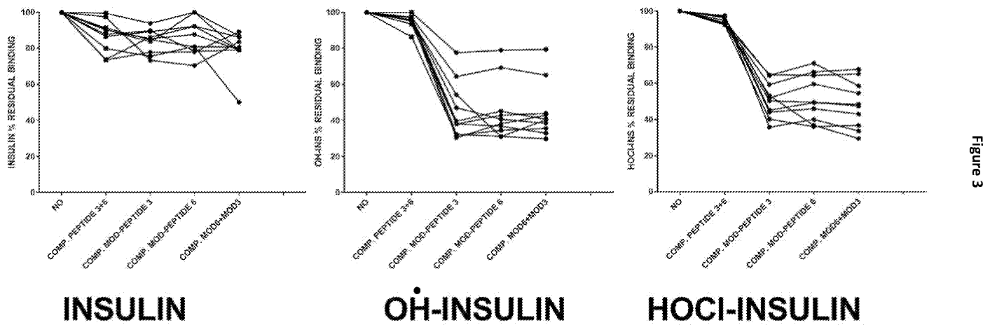

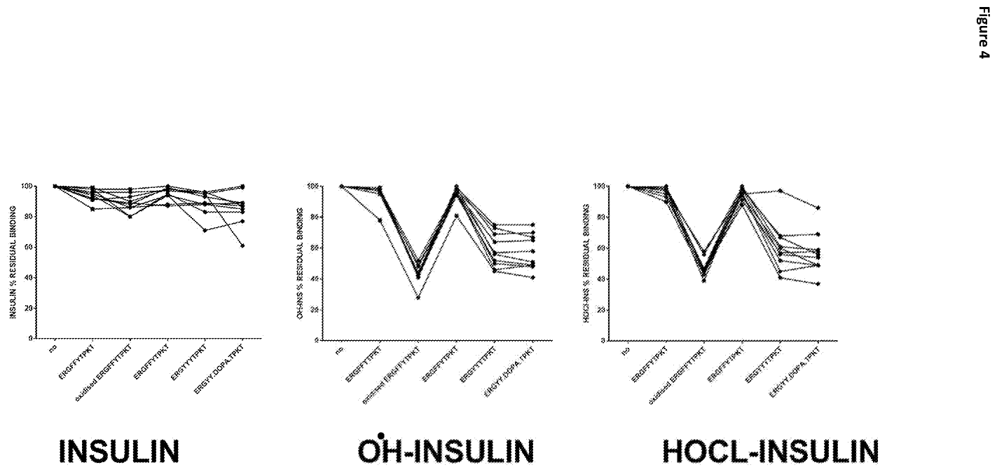

FIG. 3. Competitive ELISA for combination of in-house oxidised peptides 3 and 6
A competitive displacement assay was performed to confirm binding of autoantibodies to oxidised peptide 3 or 6 and a mixture of peptide 3 plus peptide 6 that were oxidised before mixing (comp.MOD6+MOD3). Serum samples from children with type 1 diabetes were pre-incubated with native or modified peptide 6 and 3 (COMP.PEPTIDE 3+6) or individually oxidised peptide 3 and 6 (comp.MOD6+MOD3) before adding to plate coated with either native or oxidised insulin. Bound autoantibodies were probed by anti-human IgG HRP conjugated. “NO” represents binding of serum samples without peptide competitor. INSULIN is plate coated with native insulin, ^{●}OH-INSULIN is plate coated with insulin oxidised by ^{●}OH and HOCl-INSULIN is plate coated with insulin oxidised by HOCl.

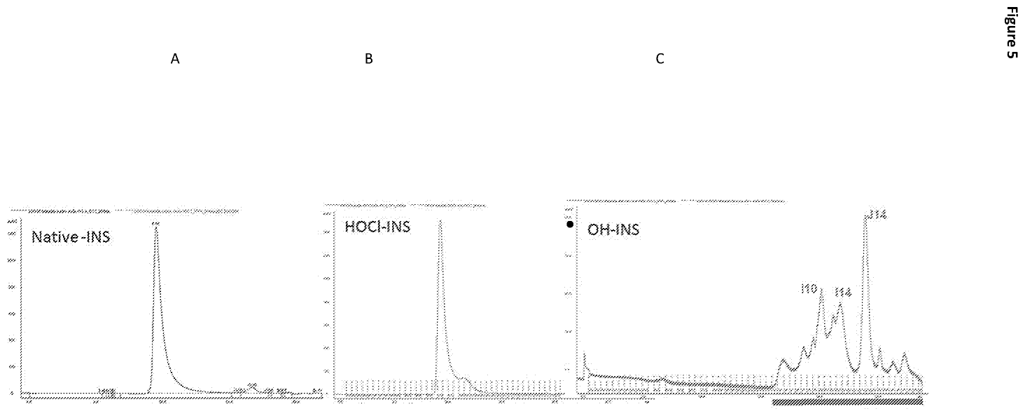

FIG. 4. Competitive ELISA for synthetically oxidised modified forms of peptide 6: (c) ERGYYYTPKT (SEQ ID NO: 6) and ERGYY-dihydroxyphenylalanine-TPKT (SEQ ID NO: 7) were used in the competition ELISA. “NO” indicates no competition, competitive peptides are given on x axis. Inhibition of binding was in the synthetically oxidised peptides were similar to the inhibition with the in-house oxidised peptide.

FIG. 5. The AKTA profile of native insulin (A) compared to insulin after exposure to HOCl (B) and ^{●}OH (C) insulin is shown. Peak fractions are after ^{●}OH modification are marked with a solid bar under the x axis.

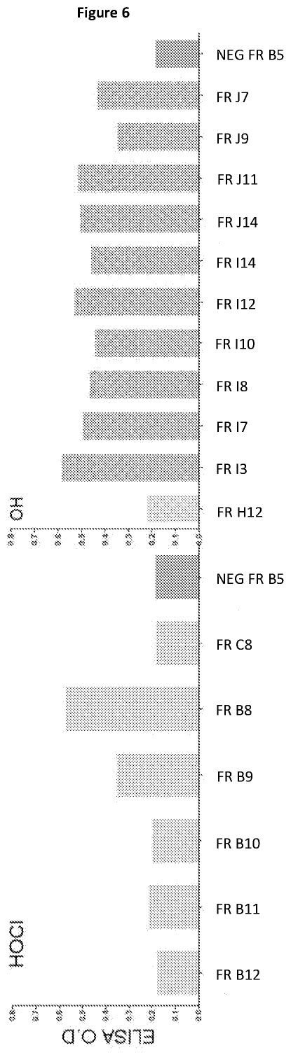

FIG. 6. ELISA of oxidised insulin fractionated by AKTA. For HOCl, the activity was in the shoulder peak fractions B9 and B8. All the peaks from the ^{●}OH modified insulin correlating to the small oxidised insulin fragments were tested positive against T1D patient sera. Fractions H12 and B5 were used as negative controls.

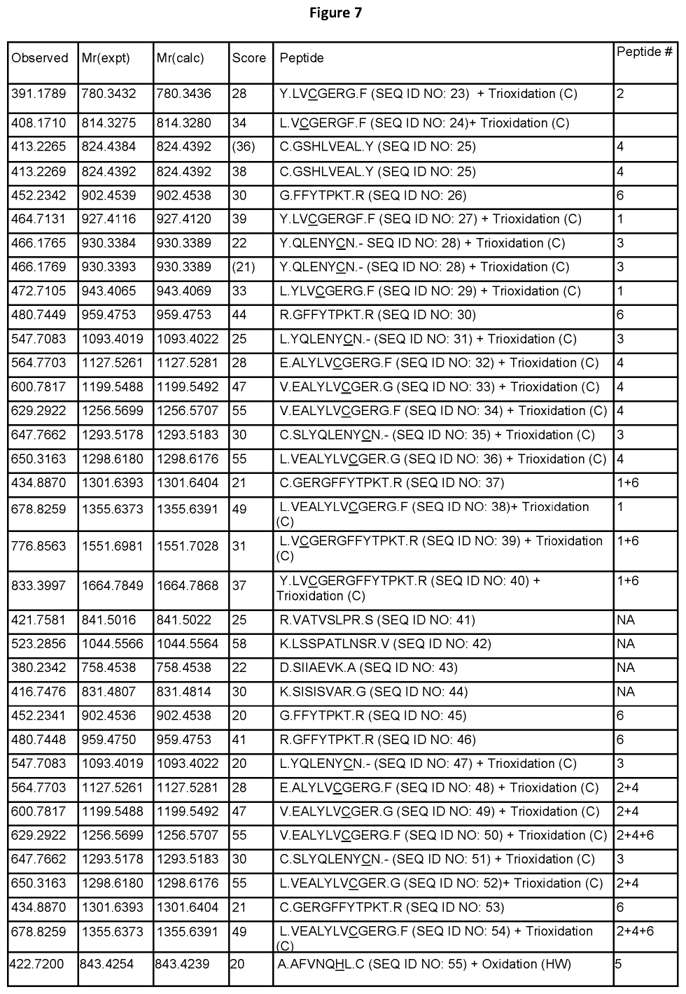

FIG. 7. Size exclusion chromatography fractions of oxPTM-INS corresponding to small insulin fragments resulting from oxPTM were collected and analysed by ELISA. Fractions that showed reactivity by ELISA were dried and analysed by LC-MS/MS. The analysis was based on the cleaved peptides following the oxidation producing singly charged ions, which are not ordinarily selected for MS/MS in a typical proteomic experiment.

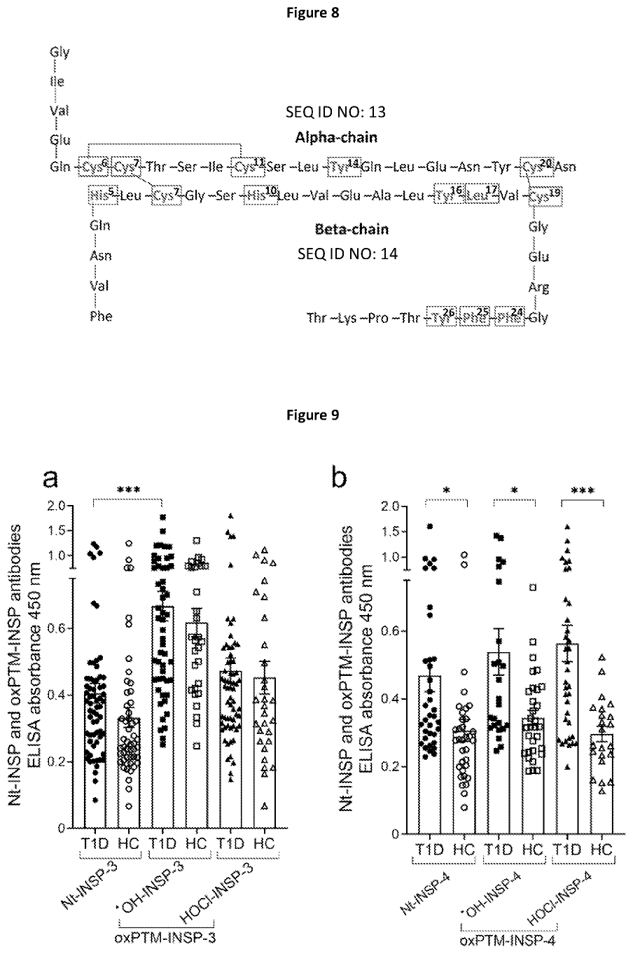

FIG. 8. Mapping the oxidized amino acids hotspot in oxidized post-translationally modified insulin (oxPTM-INS). The analyses showed that the main hotspots in oxPTM-INS involved His⁵, Cys⁷ His¹⁰, Tyr¹⁶, Leu¹⁷, Cys¹⁹, Phe²⁴, Phe²⁵, Tyr²⁶ in the insulin beta-chain. Additional hotspots were identified in the alpha-chain: Cys⁶, Cys⁷, Cys¹¹, Tyr¹⁴ and Cys²⁰. Red boxes label the newly discovered oxidation hotspots and blue boxes indicate previously described amino acid hotspots.

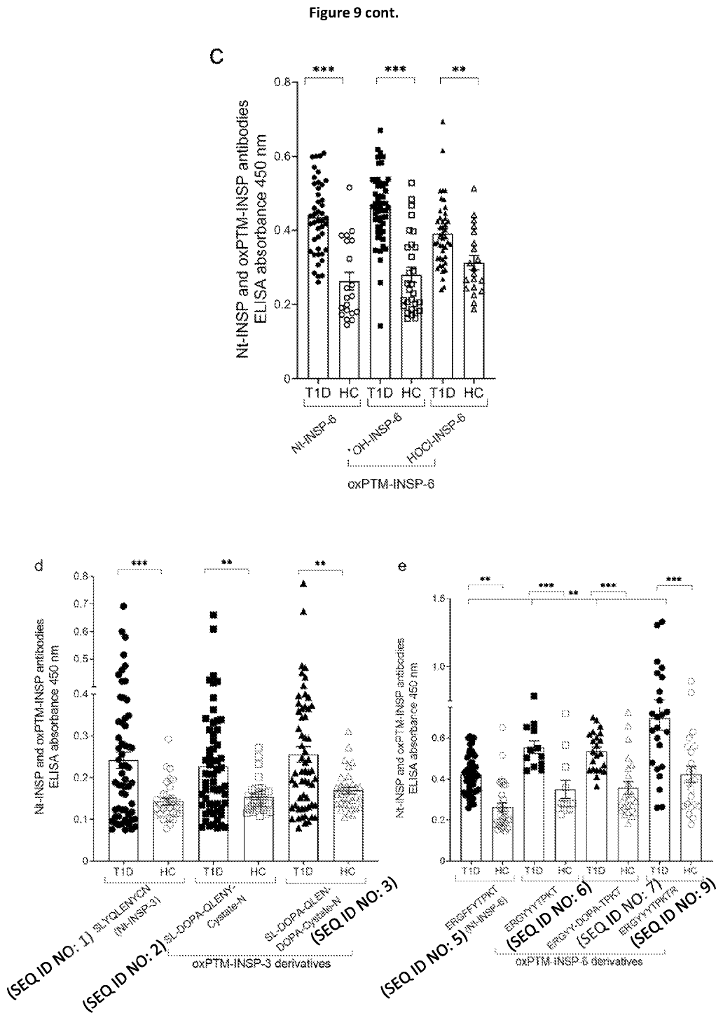

FIG. 9. Antibody binding reactivity to neo-antigenic insulin peptides (oxPTM-INSPs) in type 1 diabetes. a. After oxidation of insulin peptides by either ^{●}OH or HOCl, reactivity of type 1 diabetes serum samples against each of the native or oxPTM peptides (oxPTM-INSP) candidates were tested in ELISA. oxPTM-INSP-4 (LVEALYLVCG-ERGFFYTPKT (SEQ ID NO: 4)) and oxPTM-INSP-6 (ERGFFYTPKT (SEQ ID NO: 5)) displayed significant greater reactivity in type 1 diabetes samples compared to healthy controls (p=0.0204, p=0.0176 and p=0.0005 for native, ^{●}OH and HOCl oxPTM-INSP-4 and p<0.0001, p<0.0001 and p=0.0187, for native, ^{●}OH and HOCl oxPTM-INSP-6); oxPTM-INSP-3 showed higher reactivity compared to Nt-INSP-3 (p<0.001). The highest reactivity for oxPTM-INSP we observed was for: ^{●}OH-modified oxPTM-INSP-3 (OD=0.667±0.044), HOCl-modified oxPTM-INSP-4 (0.563±0.053) and ^{●}OH-modified oxPTM-INSP-6 (0.461±0.013). High background binding in healthy controls was observed for oxPTM-INSP-3. b. In silico oxidized oxPTM-INSP-3 derivatives of SLYQLENYCN (SEQ ID NO: 1) included: SL-DOPA-QLENY-Cysteate-N(SEQ ID NO: 2); SL-DOPA-QLEN-DOPA-Cysteate-N(SEQ ID NO: 3). In silico oxidized oxPTM-INSP-6 derivatives of ERGFFYTPKT (SEQ ID NO: 5) included ERGYYYTPKT (SEQ ID NO: 6), ERGYY-DOPA-TPKT (SEQ ID NO: 7) and ERGYYYTPKTR (SEQ ID NO: 9). Binding to SL-DOPA-QLENY-Cysteate-N(SEQ ID NO: 2) and SL-DOPA-QLEN-DOPA-Cysteate-N(SEQ ID NO: 3) was similar to SLYQLENYCN (SEQ ID NO: 1) (p=NS). In type 1 diabetes patients, binding to ERGYYYTPKT (SEQ ID NO: 6) or ERGYYYTPKTR (SEQ ID NO: 9) was higher than native ERGFFYTPKT (SEQ ID NO: 5) (p≤0.008). Similarly, significant increase in binding to ERGYY-DOPA-TPKT (SEQ ID NO: 7) was observed compared to the native ERGFFYTPKT (SEQ ID NO: 5) (p=0.008). Multiple comparisons were adjusted using the Holm-Sidak's test. p values are highlighted by asterisks: *p<0.05; **p<0.01; ***p<0.001. Cut-off points of positivity (binders) in the antibody ELISA for each peptide were defined by the mean absorbance of healthy controls to the corresponding Nt-INSP plus three times the standard error of the mean (SEM).

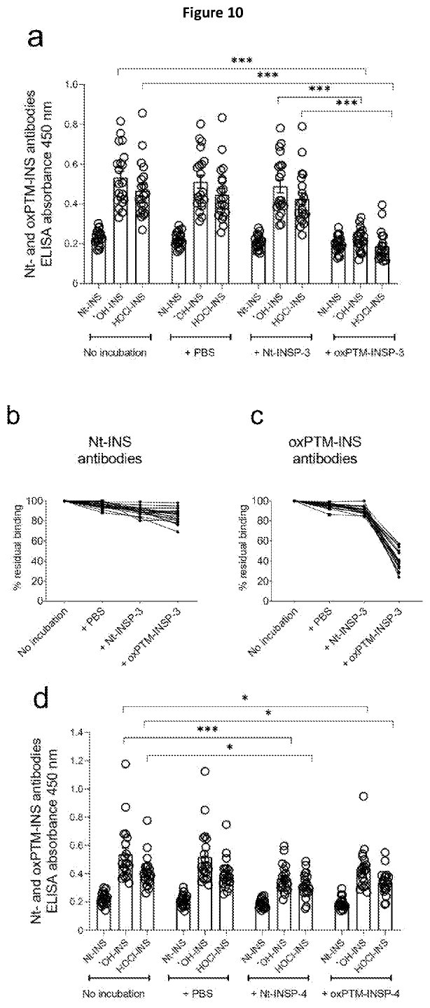

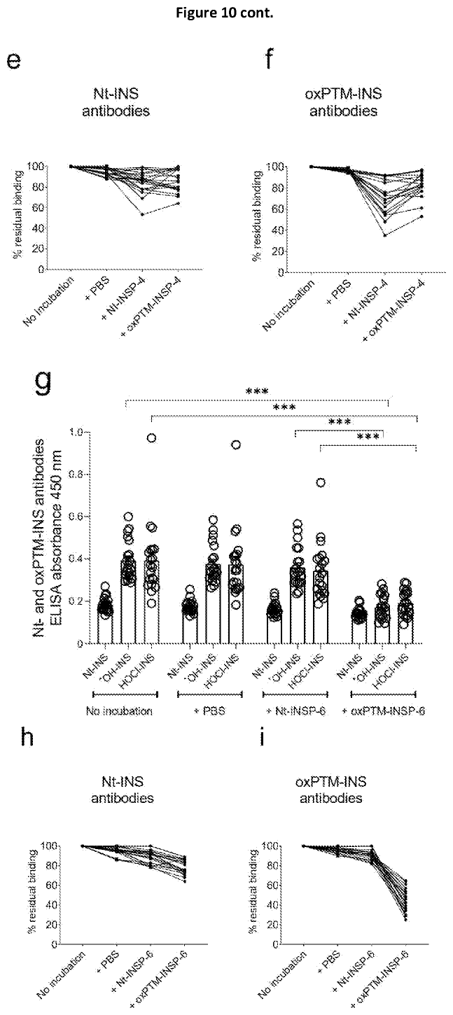

FIG. 10. Serum binding specificity to neo-antigenic insulin peptides (oxPTM-INSP). The Figure shows the residual antibody binding to Nt-INS or oxPTM-INS with and without preincubation of type 1 diabetes serum samples with native or oxPTM-INSP-3 (a, b, c), native or oxPTM-INSP-4 (d, e, f) or native or oxPTM-INSP-6 (g, h, i). Pre-incubation of type 1 diabetes serum samples with oxPTM-INSP-3 and oxPTM-INSP-6, but not with unmodified native peptides, strongly inhibited binding to oxPTM-INS (p<0001), indicating the presence of antigen-binding sites specific to these oxPTM-INSPs. Native insulin peptide 4 (Nt-INSP-4), displayed a comparable inhibition as oxPTM-INSP-4. Percent residual binding is shown for each tested type 1 diabetes sample binding to Nt-INS (b, e, h) and oxPTM-INS antibodies (c, f, i). Antibodies to ^{●}OH-INS are used as an example for oxPTM-INS. Each line in the Figure panels represent a percent binding of a serum sample from a single donor with type 1 diabetes to either Nt-INS (b, e, h) or oxPTM-INS (c, f, i) after preincubation with Nt-INSP3 or oxPTM-INSP3 (b, c); Nt-INSP-4 or oxPTM-INSP4 (e, f); Nt-INSP-6 or oxPTM-INSP6 (h, i) relative to binding to Nt-INS or oxPTM-INS without peptides competitors (100%). Multiplicity was adjusted using the Holm-Sidak's test. p values are highlighted by asterisks: *p<0.05; and ***p<0.001.

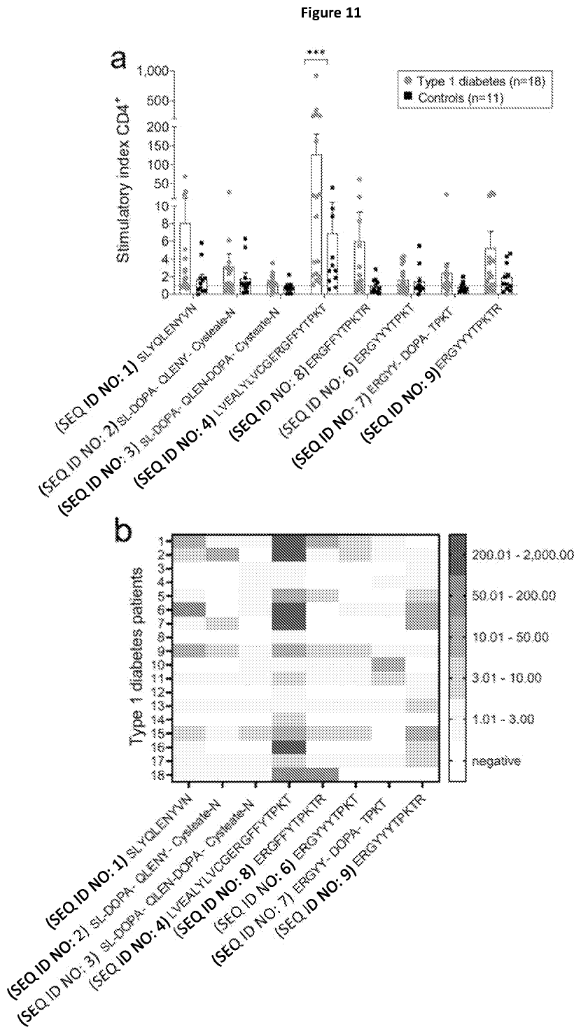

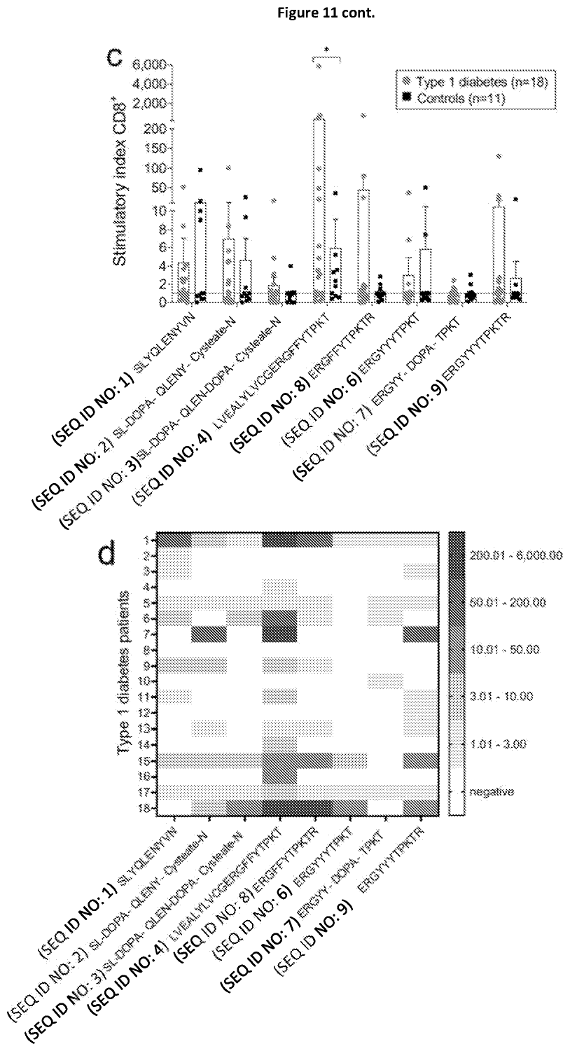

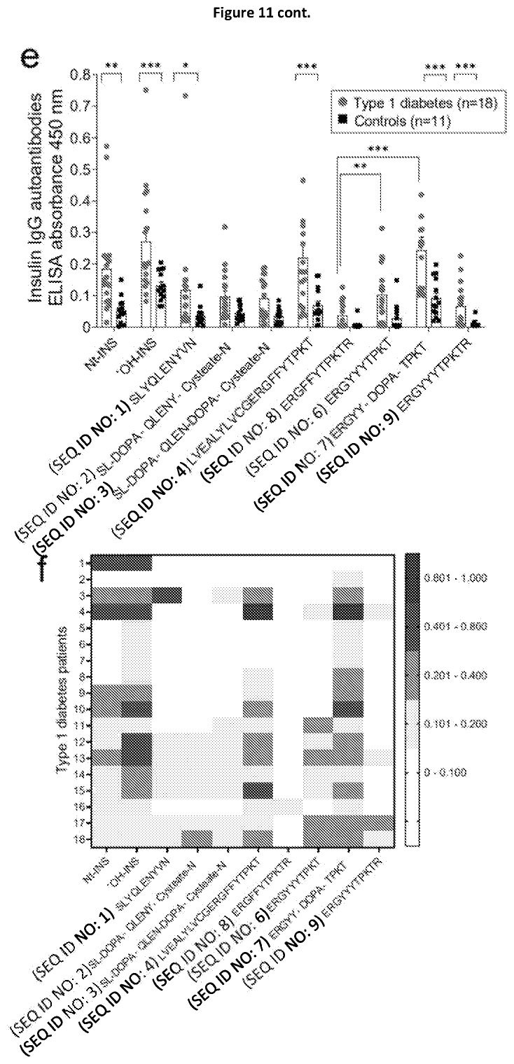

FIG. 11. oxPTM-INSPs stimulate T-cell and autoantibody responses in type 1 diabetic subject. The Figure shows the CD4⁺ (a-b), CD8⁺ (c-d) and the IgG autoantibody (e-f) responses against Nt-INS-Ps and oxPTM-INSPs. Native Peptide 4 and different oxidative derivatives of oxPTM-INSP-6 are the main targets. Heatmaps show the degree of response heterogeneity within type 1 diabetes subjects with some patients preferentially responding to different oxPTM-INSP formats derived from the same peptide sequence. CD4⁺ response (a-b, red dotted), CD8⁺ response (c-d, blue) and IgG response (e-f, green). Right panels: b, d and f show heatmap of reactivity for each individual type 1 diabetes patient tested against the various Nt-INS-Ps and oxPTM-INSPs. Multiplicity adjusted p values are highlighted by asterisks: *p<0.05; **p<0.01; ***p<0.001.

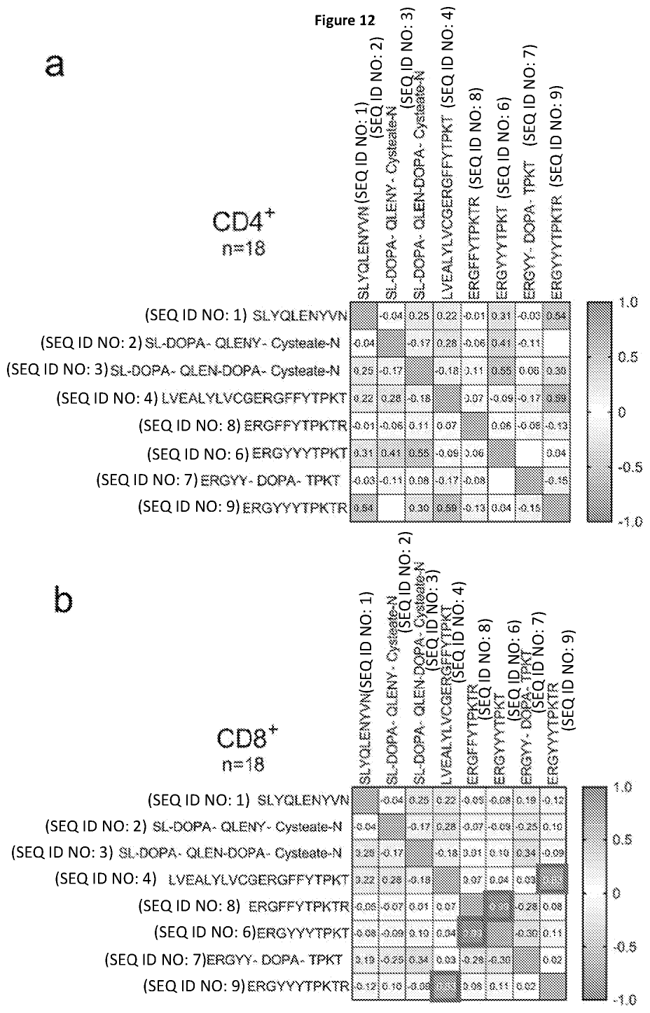

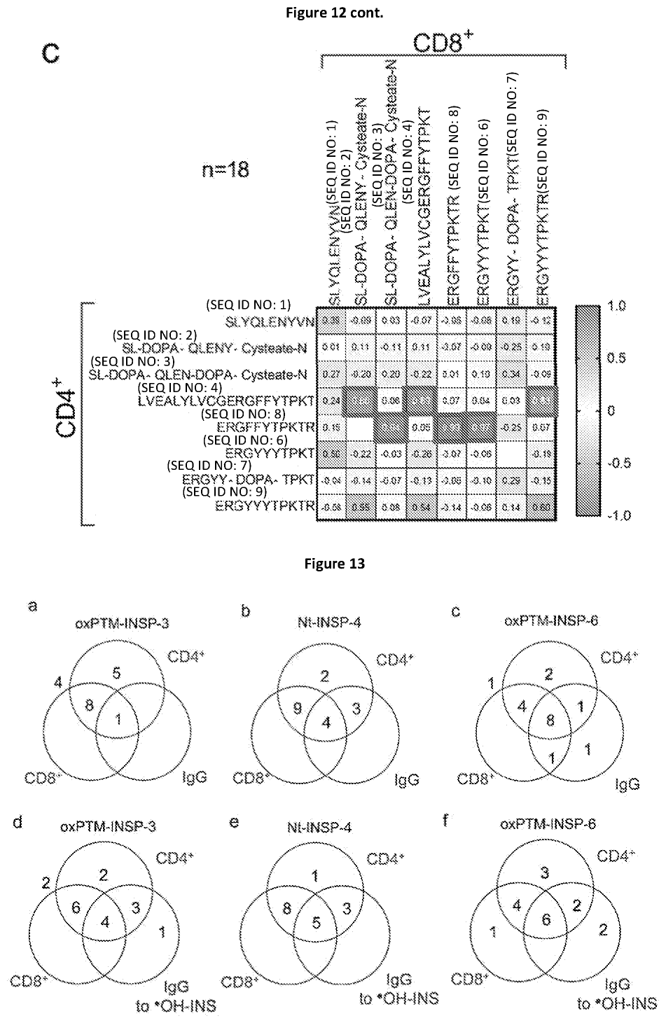

FIG. 12. Correlation matrix of CD4⁺ and CD8⁺ responses to oxPTM-INSP. T-cell stimulation with Nt-INSP-4 (LVEALYLVCGERGFFYTPKT (SEQ ID NO: 4)) strongly correlated with the ox-PTM-INSP-6, ERGYYYTPKTR (SEQ ID NO: 9) containing two aminoacidic oxidations (conversion of Phe²⁴ and Phe²⁵ to Tyr) for CD8⁺ (r=0.83, p=0.002) responses. There was no correlation between stimulation by Nt-INSP-4 and the Nt-INSP-6 (ERGFFYTPKTR (SEQ ID NO: 8)) containing the native aminoacidic sequence (a and b). CD4⁺ stimulatory response to Nt-INSP-4 correlated with CD8⁺ responses to Nt-INSP-4 (r=0.89; p=0.0017), oxPTM-INSP-6 (ERGYYYTPKTR (SEQ ID NO: 9)) (r=0.83; p=0.002) and oxPTM-INSP-3 (SL-DOPA-QLENY-Cysteate-N(SEQ ID NO: 2)) (r=0.86; p=0.0018) (c). All analyses are corrected for multiple comparisons, with statistically significant correlations highlighted by green squares.

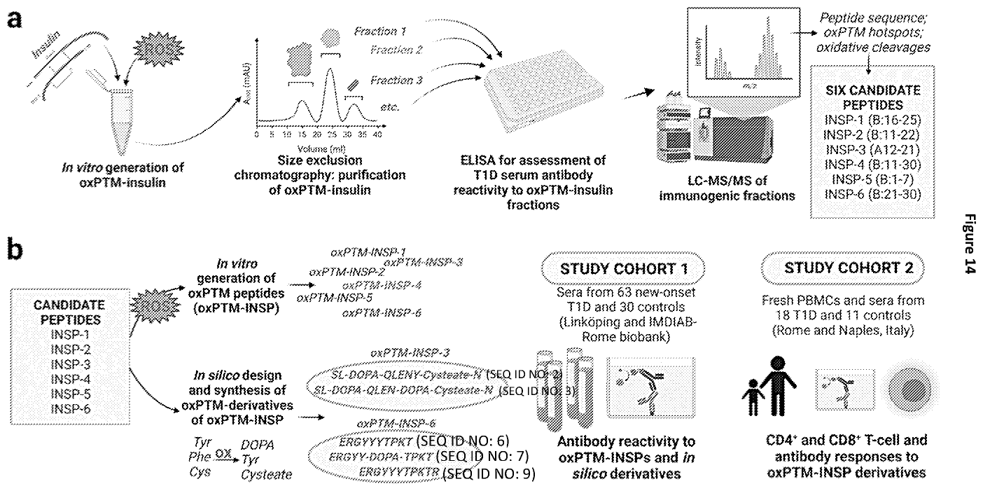

FIG. 13. Venn diagrams of CD4⁺, CD8⁺ and antibody response to oxPTM-INSPs. Panels a, b, and c show the overlap between IgG autoantibody and T-cell (CD4⁺ and CD8⁺) responses specific to oxPTM-INSP-3 (a), Nt-INSP-4 (b) and oxPTM-INSP-6 (c). Panels d, e, and f display the overlap between T-cell responses to the three insulin peptides and IgG to oxPTM-INS modified by hydroxyl radical (^{●}OH-INS). Overall, 5.5%, 22.2%, and 44.4% of patients had a concordant response involving simultaneously CD4⁺, CD8⁺ and IgG towards oxPTM-INSP-3, Nt-INSP-4 and oxPTM-INSP-6, respectively. There is concordance between reactivity to oxPTM-INS and CD4⁺ and CD8⁺ reactivity for all three tested peptides (7/17, 8/18 and 8/18 for oxPTM-INSP-3, Nt-INSP-4 and oxPTM-INSP-6, respectively). The stimulatory index >1 was used for definition of positive T-cell response.

FIG. 14. Epitope mapping strategy. a Oxidative posttranslational modifications (oxPTM) of insulin were generated using various reactive oxidants (ROS). Peptide fractions of oxPTM-insulin (oxPTM-INS) were separated by size exclusion chromatography, and immunogenic fractions further characterized by LC/MS-MS. b. The identified peptide candidates were then oxidized in house (oxPTM-INS peptides), or in silico modified to generate multiple oxPTM-INS peptides derivatives corresponding to one or more aminoacidic modifications. The A:12-21 peptide SLYQLENYCN (SEQ ID NO: 1) (Nt-INSP-3) was oxidized to the oxPTM-INSP-3 derivatives SL-DOPA-QLENY-Cysteate-N(SEQ ID NO: 2) and SL-DOPA-QLEN-DOPA-Cysteate-N(SEQ ID NO: 3); the B:21-30 peptide ERGFFYTPKT (SEQ ID NO: 5) (Nt-INS-P6) was oxidized to the oxPTM-INSP-6 derivatives ERGYYYTPKT (SEQ ID NO: 6), ERGYY-DOPA-TPKT (SEQ ID NO: 7), and ERGYYYTPKTR (SEQ ID NO: 9). Autoantibody reactivity to the Nt-INSP and oxPTM-INS peptides was tested using sera from 63 subjects with new-onset type 1 diabetes (T1D) (Linkoping and IMDIAB biobanks). In a second cohort we evaluated, in parallel, CD4⁺ and CD8⁺ T-cell and autoantibody responses to the oxPTM-INS peptides derivatives by using fresh PBMCs and sera collected from 18 recent onset and 5 additional established T1D patients.

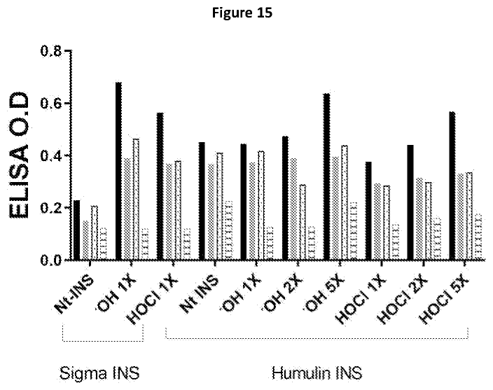

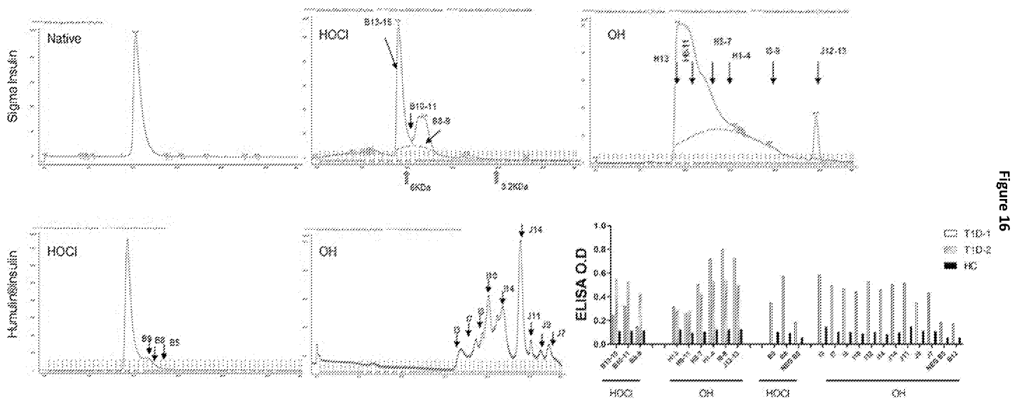

FIG. 15. Reactivity pattern of type 1 diabetes samples to oxPTM Humulin R® insulin versus Sigma oxPTM-INS. We show reactivity for three type 1 diabetes (T1D) patients (black, grey and dotted bars) and compared it to one healthy control (strike pattern). The term 1×^{●}OH represent exposure of insulin to 4.5 mM CuCl₂ and 9 mM hydrogen peroxide (modification introduced by the Fenton reaction). 1×HOCl means exposure to 2 mM HOCl. Different oxidation conditions were tested to optimized for Humulin R® insulin that was formulated differently by the manufacturer. The terms “2×” and “5×” denotes the scaled-up of the above treatment conditions. Humulin R® oxPTM displays similar reactivity as for the Sigma insulin.

FIG. 16. The size exclusion chromatography ÄKTA. Superdex 30 column suitable for the detection of low molecular weights (100-7000 kDa) was used for separating the various insulin fragments that were obtained after the oxidative posttranslational modification (oxPTM) of either Sigma insulin or Humulin R® insulin. Sigma and Humulin R® native insulin (Nt-INS) display a single peak at around 10 ml. Following oxidation, oxPTM-INS different small (<6 kDa) insulin fragments were observed. Sigma insulin exposed to HOCl showed a major peak at around 10 ml with additional peak at lower molecular weight. Exposing Sigma insulin to ^{●}OH resulted in a broad peak of small molecular fragments <6 kDa at elution volumes between 10 ml and 25 ml, and an additional peak at 25 ml. Humulin R® insulin, subjected to oxidation with HOCl displayed a major peak at 10 ml with an additional shoulder of a smaller molecular weight. For ^{●}OH modification of Humulin R® insulin, the major native peak corresponding to native insulin disappeared but instead we observed a set of multiple peaks towards the end of the chromatograph corresponding to small molecular weights <6 kDa. c. Type 1 diabetes (T1D) serum samples but not healthy controls (HC) bound to the small molecular weight fractions that resulted from either HOCl or ^{●}OH modification.

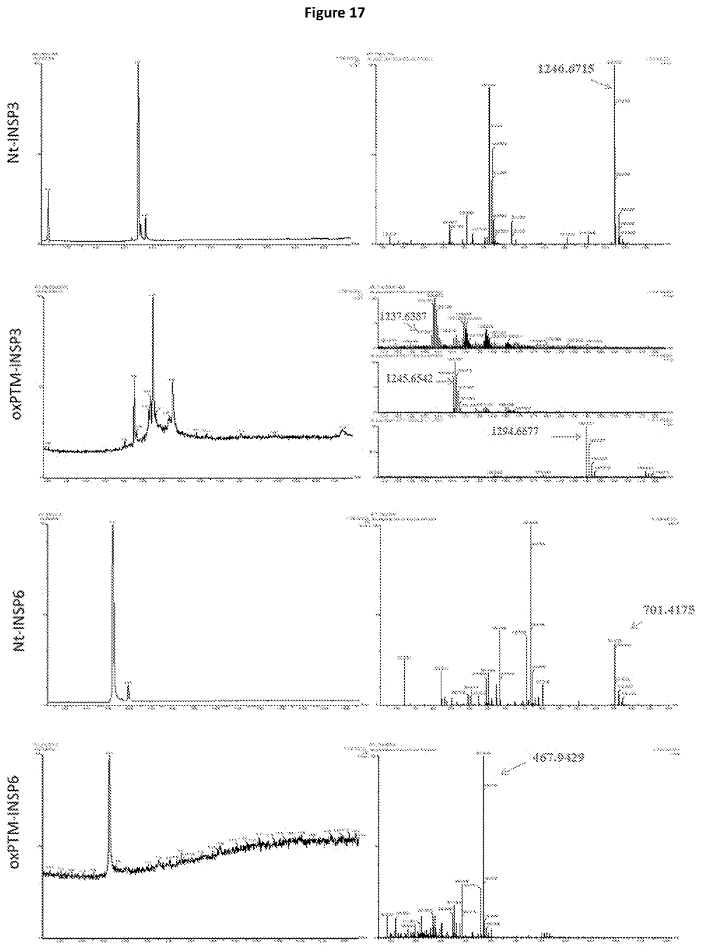

FIG. 17. Testing peptides oxidation by TOF-MS/ES+TIC. Peptide candidates were exposed to either HOCl or ^{●}OH and were assessed by TOF-MS/ES+TIC. Native insulin peptide 3 (Nt-INSP3) display a single ionization peak corresponding to SLYQLENYCN (SEQ ID NO: 1) [R—NH3]+, m/z 1246.6751. oxPTM-INSP-3 corresponding to m/z 1237.6387, 1245.6542 and 1294.6677 a shift in mass as an indication of oxidative modification (potentially corresponding to addition of single or multiple OH (16 Da). Native insulin peptide 6 (Nt-INSP6, ERGFFYTPKTR (SEQ ID NO: 8), m/z 1401.57) display a single LC-MS/MS peak at 701.4175 corresponding to double ionization mass. After oxidation, there was significant degradation and only a single peak of 467.9429 m/z is seen.

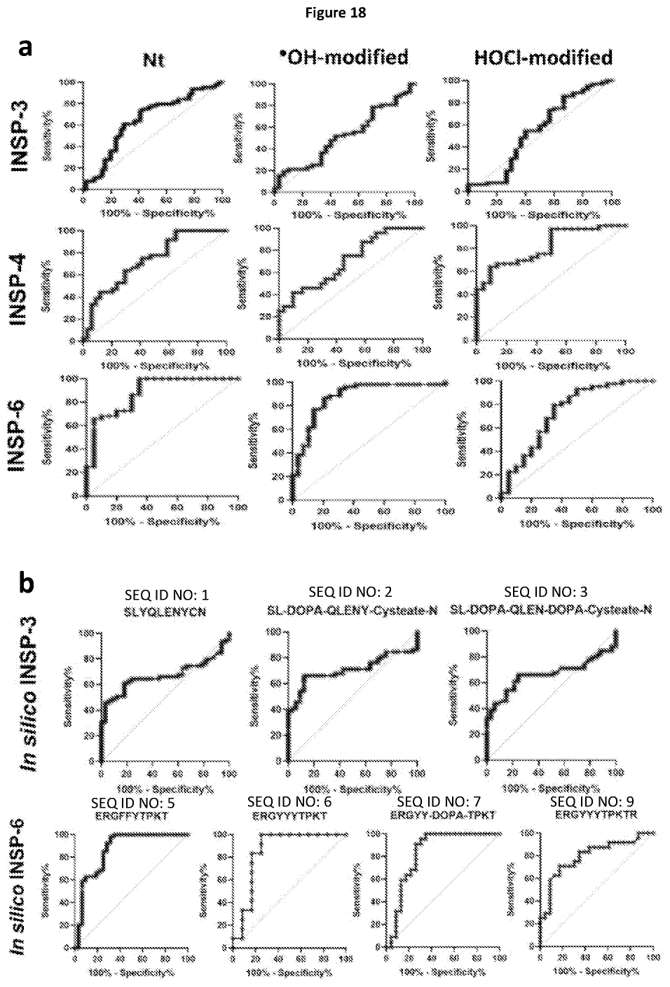

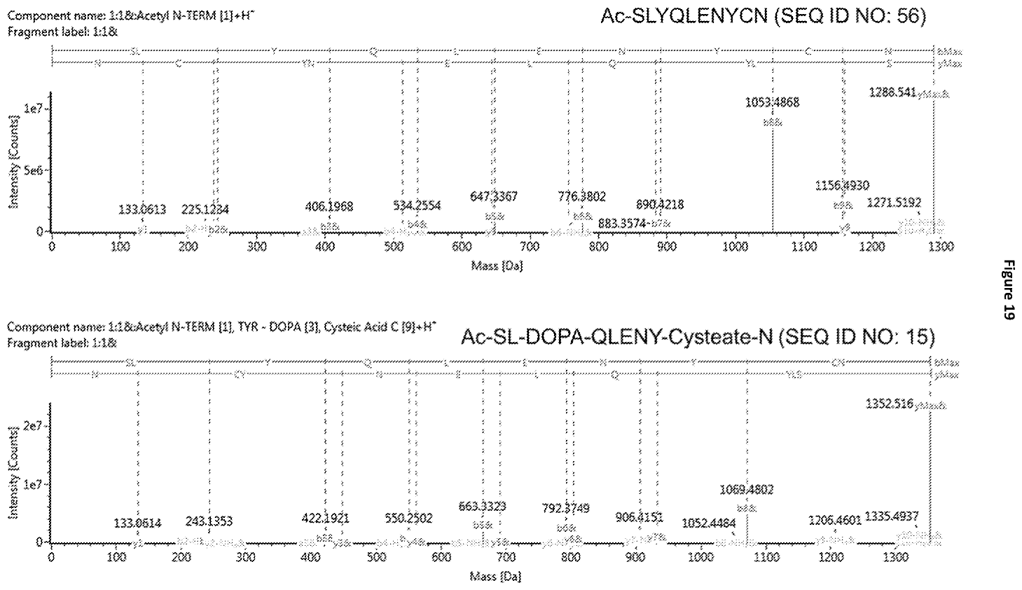

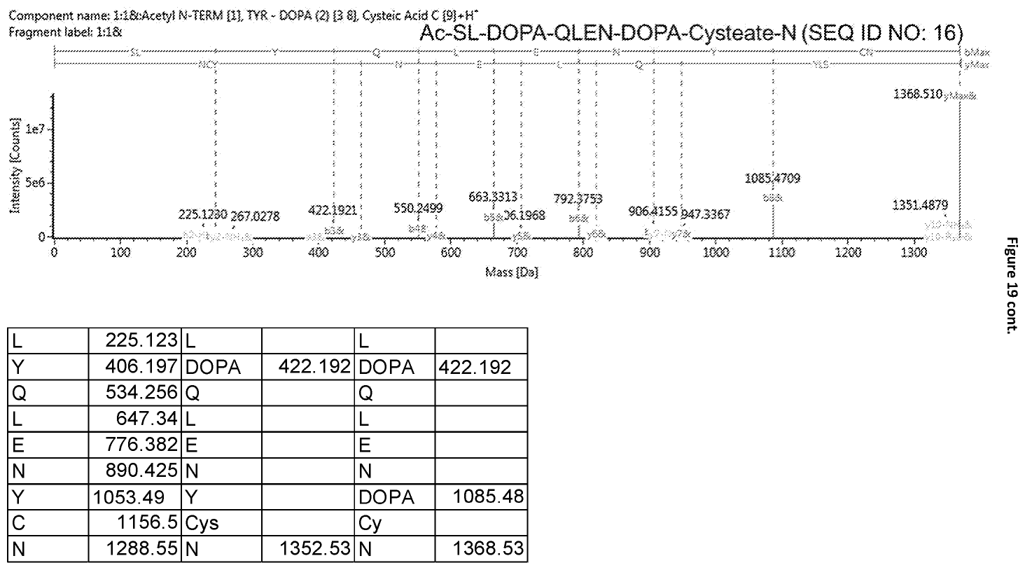

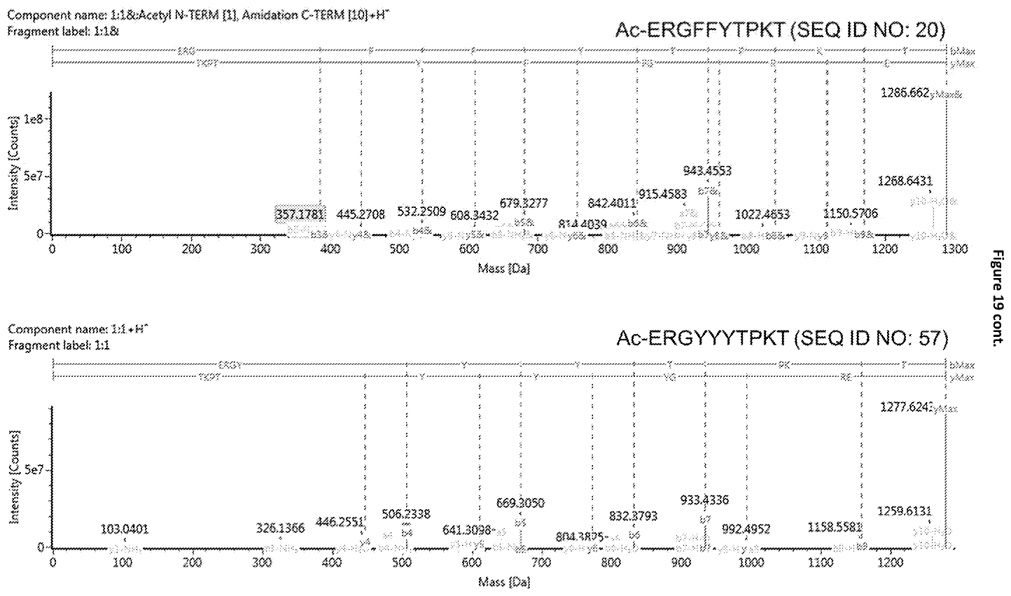

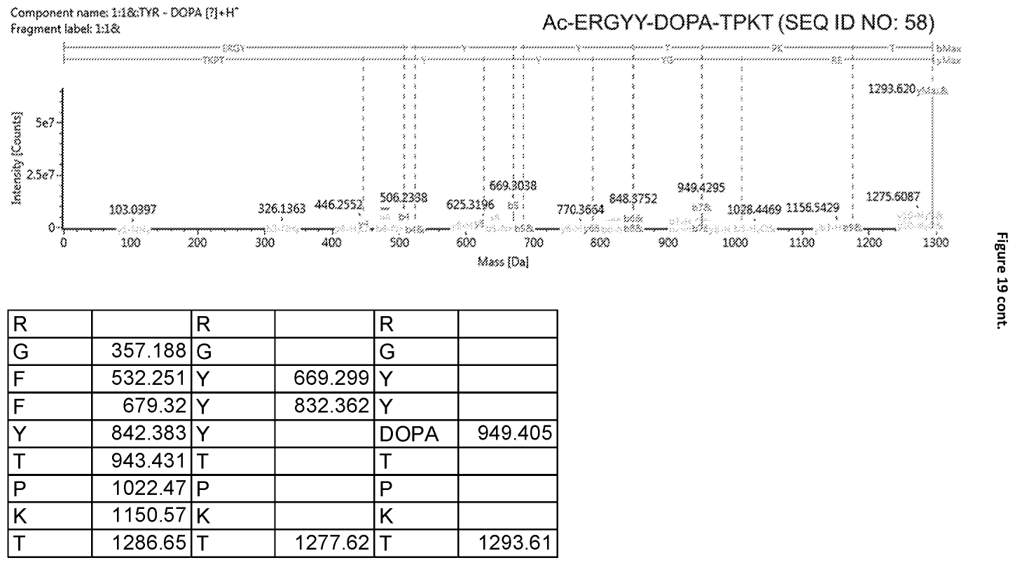

FIG. 18. ROC Curve analysis for insulin peptides INSP-3, INSP-4 and INSP-6, native and modified in house (a) or in silico (b). The highest specificity/sensitivity observed is for peptide 6 whether it was oxidized in house by ^{●}OH or HOCl, or in silico oxidized, which showed ROC-AUC>0.8 for all peptide versions.

FIG. 19. Peptide sequence confirmation by UPLC-qTOF/MS^{e}. Fragmentation spectra of the three versions of peptides 3 (INSP-3) and 6 (INSP-6) is shown. INSP-3 has common sequence backbone, SLYQLENYCN (SEQ ID NO: 1) [R—NH3]⁺, m/z 1288.551), Ac-SL-DOPA-QLENY-cysteate-N(SEQ ID NO: 15); [R—NH3]⁺, m/z 1352.5162) and Ac-SL-DOPA-QLEN-DOPA-cysteate-N(SEQ ID NO: 16); [R—NH3]⁺, m/z 1368.5091). The fragmentation pattern of native INSP-6 and oxPTM-INSP6, ERGFFYTPKT (SEQ ID NO: 5)-NH2; [R—NH3]⁺, m/z 1286.6626); ERGYYYTPKT (SEQ ID NO: 6); [R—NH3]+, m/z 1277.6253) and ERGYY-DOPA-TPKT (SEQ ID NO: 7); [R—NH3]+, m/z 1293.6213 is also shown. Table highlights the fragment ions identified with different m/z as a result of modification.

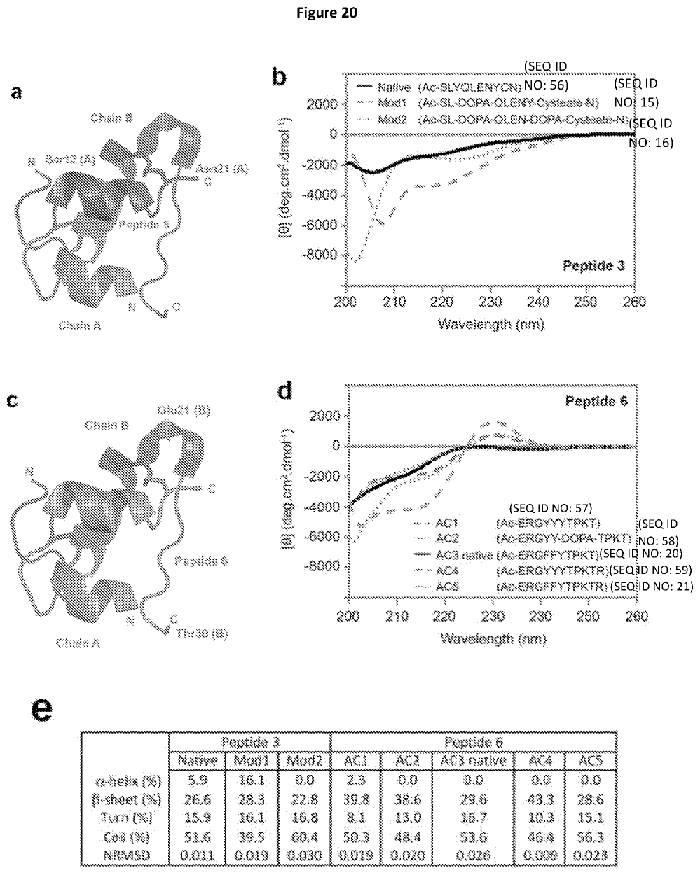

FIG. 20. Structural changes in the neo-antigenic insulin peptides. a. Structure of human insulin (PDB ID: 5bts) highlighting the location of peptide 3 in chain A (coloured green). b. Circular dichroism spectra for native, SL-DOPA-QLENY-cysteate-N(SEQ ID NO: 2) (Mod1) and SL-DOPA-QLEN-DOPA-cysteate-N(SEQ ID NO: 3) (Mod 2) forms of peptide 3. c. Structure of human insulin highlighting the location of peptide 6, Ac-ERGFFYTPKT (SEQ ID NO: 20) within chain B (coloured green). d. Circular dichroism spectra of native Ac-ERGFFYTPKT (SEQ ID NO: 20) (AC3) and Ac-ERGFFYTPKTR (SEQ ID NO: 21) (AC5) versus oxPTM-INSP-6 ERGYYYTPKT (SEQ ID NO: 6) (AC1), ERGYY-DOPA-TPKT (SEQ ID NO: 7) (AC2) or ERGYYYTPKTR (SEQ ID NO: 9) (AC4). e. Secondary structure analysis of the circular dichroism data using BeStSel implies significant structural changes of peptides upon oxPTM but which are not correlated with the degree of modification.

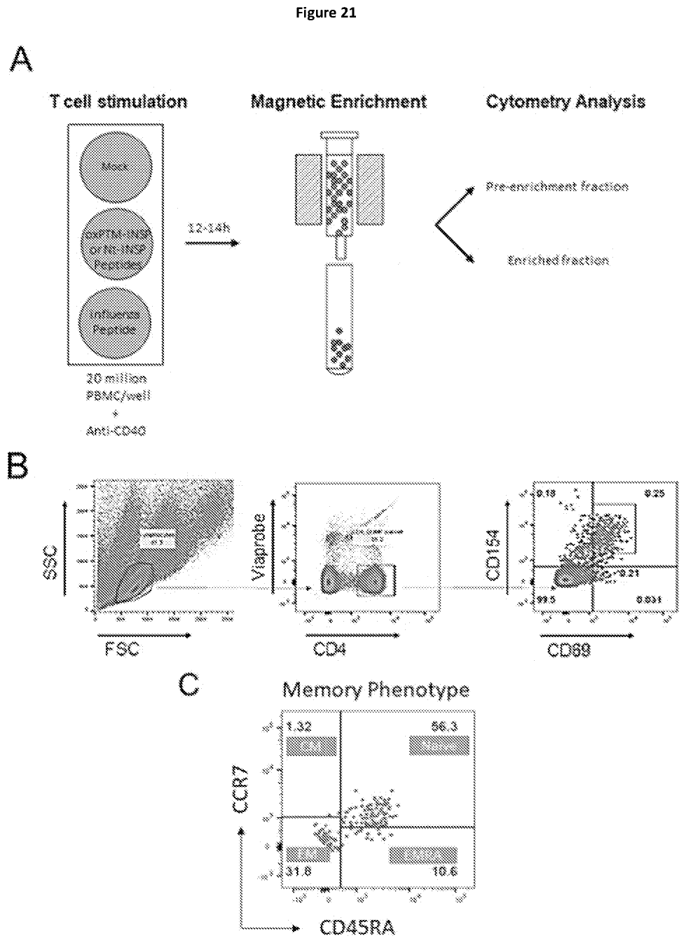

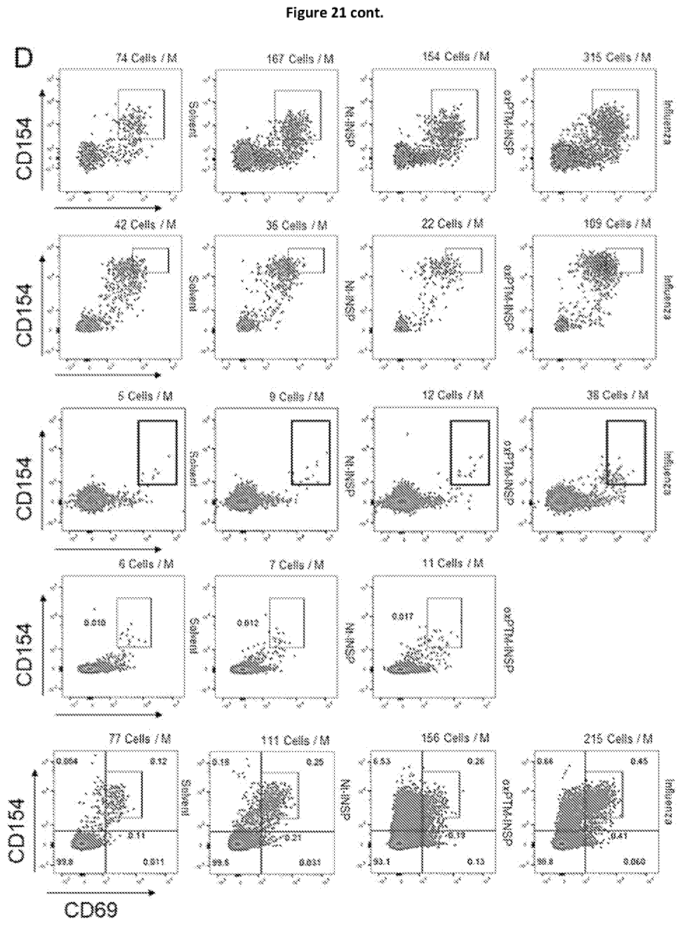

FIG. 21. Strategy for CD154 based detection of CD4+ T cell responses. (A) The surface marker upregulation assay consists of a short-term activation of PBMC with solvent only (Mock) or specific peptides of interest (oxPTM-INSP, Nt-INSP, or an influenza peptide) in individual wells for 10-14 hours in the presence of an anti-CD40 blocking antibody to prevent downregulation of CD154. The recently activated CD154+ cells are then magnetically labeled and then enriched over an MS column, reserving a pre-enriched fraction to estimate the total number of CD4+ T cells in the sample. Both the pre-enriched and enriched samples are then analyzed by flow cytometry. (B) The gating scheme for the CD154 assay is to select lymphocytes on a forward scatter (FSC) versus side scatter (SSC) plot, select live CD4+ T cells on a CD4 versus Viaprobe plot, and then select and count CD154+CD69+ cells as the recently activated antigen specific population. (C) A biaxial plot of CD45RA versus CCR7 was gated based on total CD4+ T cells and the utilized to partition CD154+ cells into naïve (CD45RA+CCR7+), TCM (CD45RA−CCR7+), TEM (CD45RA−CCR7−), and TEMRA (CD45RA+CCR7−) phenotypes. (D) Surface marker upregulation results obtained for five subjects with T1D are all shown. In some subjects, the frequency of CD154+CD69+ (epitope specific) T cells was higher for native insulin peptides, oxidized insulin peptides, and the influenza peptide than the solvent only control.

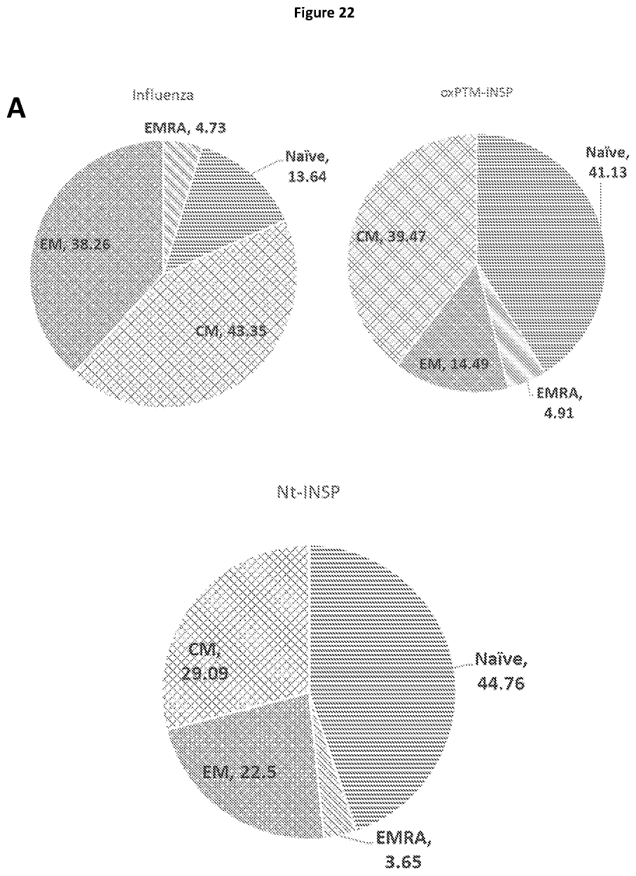

FIG. 22. CD4+ T cell responses toward native and oxidized insulin peptides. Across the five T1D subjects assayed, we utilized surface staining for CD45RA and CCR7 on CD154+CD69+ T cells to classify epitope specific T cells as Naïve (CD45RA+CCR7+), TCM (CD45RA−CCR7+), TEM (CD45RA−CCR7−) or TEMRA (CD45RA+CCR7−). (A) Averaging across the five subjects, influenza specific T cells were predominantly TCM and TEM as expected. Native insulin and oxidized insulin specific T cells trended toward having a higher percentage of naïve cells (44.7% and 41.1% respectively) but also had notable proportions of TCM and TEM, suggesting that there is an existing pool of memory T cells that recognizes these insulin peptides in subjects with T1D. (B) Plotting the same data as individual data points for each subject preserved the same tendencies, but reveals some heterogeneity in the relative proportion of Naïve, TCM, and TEM cells.

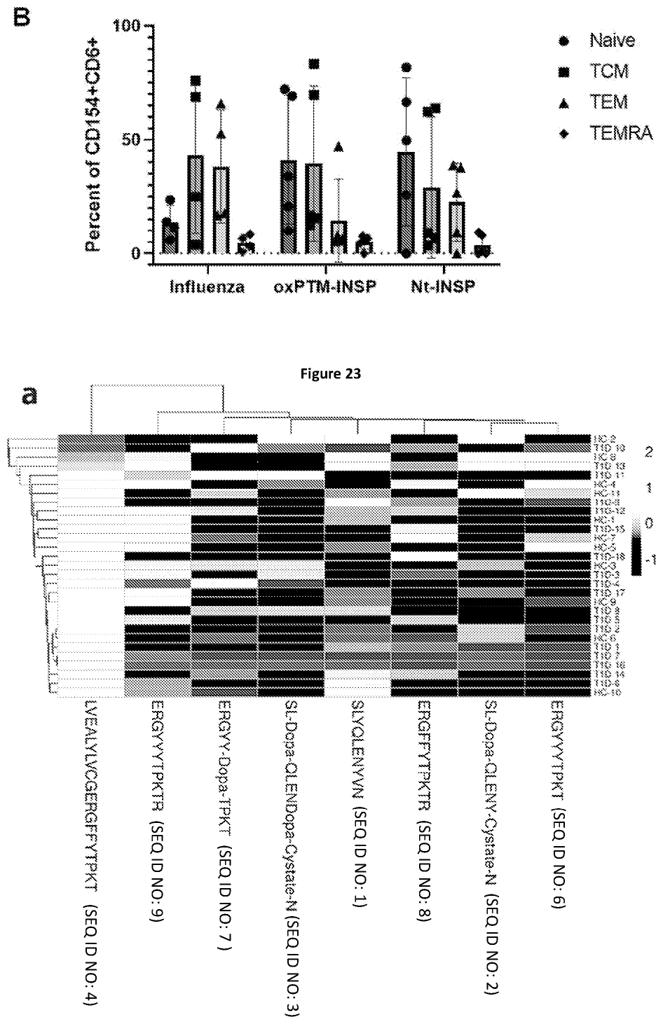

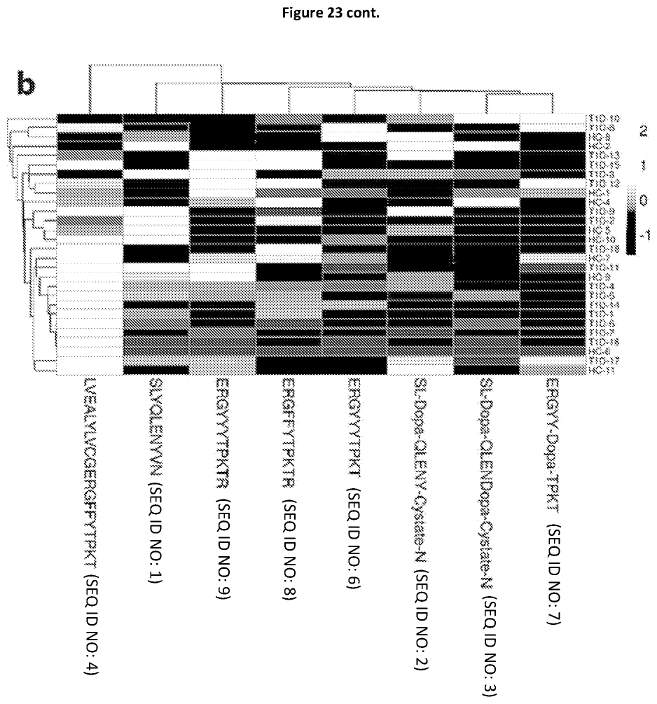

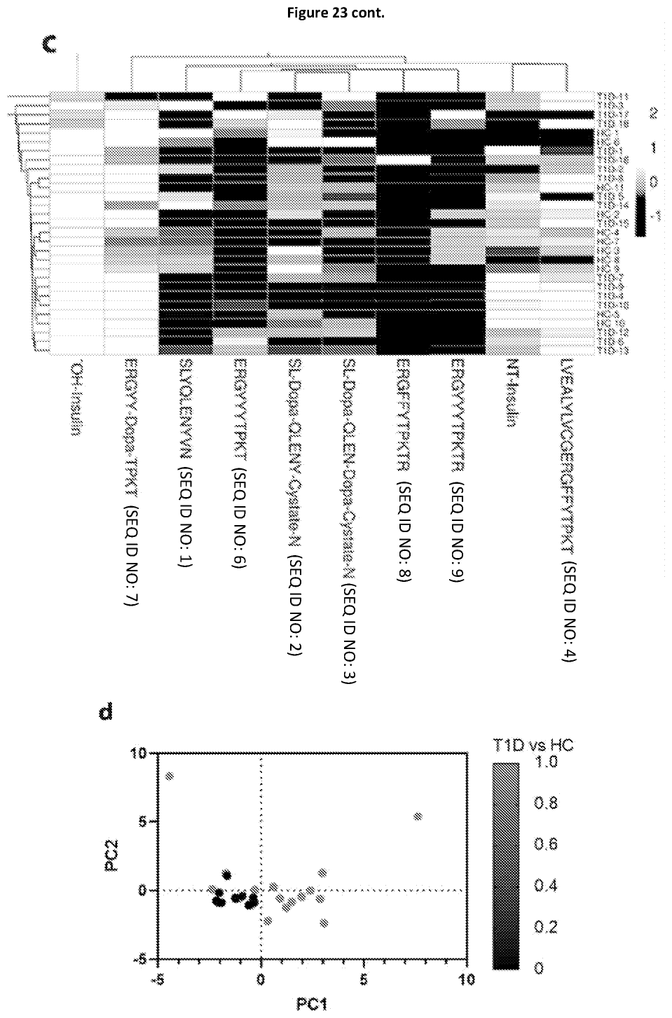

FIG. 23. Hierarchical cluster analysis. Hierarchical cluster analysis and principal component analysis (PCA) revealed association between the responses to different oxPTM-INSP and identify clustering of type 1 diabetes versus healthy control samples. We observed association between Nt-INSP-4 and ox-PTM-INS-P6, ERGYYYTPKTR (SEQ ID NO: 9) for CD4⁺ (a) and CD8⁺ (b). For CD4⁺ response Nt-INSP-4 is also closely associated to ERGYY-DOPA-TPKT (SEQ ID NO: 7). For IgG response (c) ERGYY-DOPA-TPKT (SEQ ID NO: 7) is associated with ^{●}OH-Insulin. We also observed clustering of response of type 1 diabetes samples using PCA analysis of all responses, CD4, CD8 and IgG (d). We observed cluster of 11 type one diabetes samples with PC1>0 while the rest clustered with healthy control samples with PC1<0.

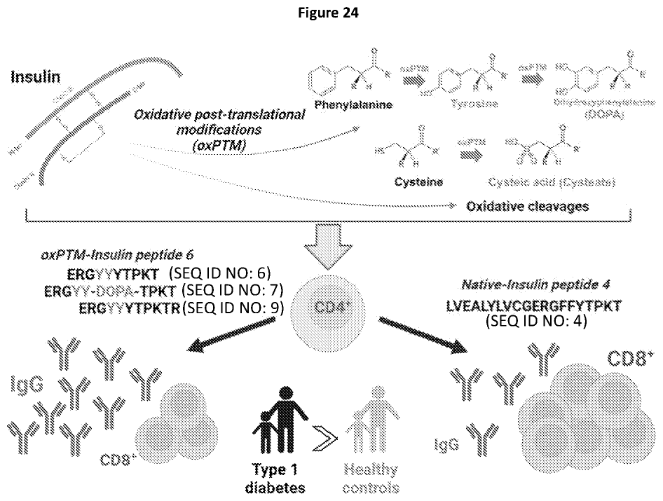

FIG. 24. Graphic overview of the generation and testing of oxidative post-translational modifications (oxPTM) of insulin (oxPTM-INS) peptides. Antibodies specific to oxidative post-translational modifications (oxPTM) of insulin (oxPTM-INS) are present in individuals with type 1 diabetes. Antibody responses to oxPTM-INS neoepitope peptides (oxPTM-INSP) were investigated and evaluated for their ability to stimulate humoral and T-cell responses in type 1 diabetes.

### EXAMPLES

### Example 1—Analysis of Oxidised Insulin Fragments by Size Exclusion Chromatography

The inventors tested a cohort of patients with T1D early in the onset for their reactivity to both native and oxidised insulin peptides. The later were exposed to reactive oxidants such as hydroxyl radicals (^{●}OH), hypochlorous acid (HOCl), and ribose. The structural modification was then analysed AKTA and mass spectrometry (MS).

The inventors have used size exclusion chromatography using the AKTA purifier system (GE healthcare) to monitor the molecular profile of native versus oxidised insulin, collect the various fractions and test their activities by ELISA further to MS analysis. We used Superdex 30 increase 10/30 GE Healthcare that is suitable for detecting low molecular weights of 100-7000 Da (FIG. 17). For native insulin, we observed a single peak at around 10 ml which corresponded to 5900 Da of insulin. Contrastingly, with the ^{●}OH and HOCl modification, the major native peak corresponding to native insulin disappeared but instead we observed a set of multiple peaks corresponding to small fragment towards the end of the column corresponding to small molecular weight (lower than 3 kDa).

Fractions correlating from individual peaks were collected for both ELISA. For HOCl insulin we collected fractions corresponding to native insulin fractions B (B10-B12) and the shoulder peak (B9 and B8). For ^{●}OH insulin we collected: I2, I3, I4, I5, I6, I7, I8, I9, I10, I11, I12, I13, I14, I15, J15, J14, J13, J12, J11, J10, J9, J8, J7, J6, J5 (FIG. 17). ELISA showed reactivity to oxidised insulin in fraction B8 which corresponds to the shoulder peak of HOCl but not to fractions B10-B12 of native insulin. For the ^{●}OH modification, we observed reactivity to all the peaks corresponding to the small molecular weight modified fragments (FIG. 17).

### Example 2—Analysis of Oxidised Insulin Fragments by Mass Spectrometry

AKTA collected fractions that showed reactivity in ELISA were dried down and resuspended in around 30 μl of 0.1% formic acid. The fractions were then analysed by LC-MS/MS (Orbitrap Velos, Thermo Scientific) at the Cambridge university UK core facility. The analysis has been more complex than we first anticipated due to the fact that ^{●}OH oxidation resulted in cleaved peptides that are relatively small and produce singly charged ions which are not ordinarily selected for MS/MS in a typical proteomic experiment. Secondly, the lack of specificity of the cleavage point makes the database searching more challenging. In a typical proteomic experiment, proteins are digested with specific enzymes such as trypsin and so the peptide cleavage sites are very well defined but in the current experiment, we are relying on the breakdown of the protein due to oxidation, which is a much more random. We are pleased to report that we found a way to overcome the problems and that the amino acid sequence of the active peaks following ^{●}OH modification, J12-14, I12-I14 and J9-J11 is ERGFFYTPKTR (SEQ ID NO: 8). Additional sequence that was identified with less hits and scoring is AAFVNQHLC (SEQ ID NO: 22). The amino acid sequence ERGFFYTPKTR (SEQ ID NO: 8) is at the C terminus end of the beta chain while AAFVNQHLC (SEQ ID NO: 22) is at the N terminus of beta chain. Additional AKTA fractionation and MS analysis identified in addition peptides from the beta chain that included; YLVCGERGFF (SEQ ID NO: 10); LVEALYLVCGER (SEQ ID NO: 11); and LVEALYLVCGERGFFYTPKT (SEQ ID NO: 4). One peptide was identified from the alpha chain: SLYQLENYCN (SEQ ID NO: 1).

In total MS identified 6 peptides:

(a) (Peptide 1)
(SEQ ID NO: 10)
YLVCGERGFF;
(b) (Peptide 2)
(SEQ ID NO: 11)
LVEALYLVCGER;
(c) (Peptide 3)
(SEQ ID NO: 1)
SLYQLENYCN;
(d) (Peptide 4)
(SEQ ID NO: 4)
LVEALYLVCGERGFFYTPKT;
(e) (Peptide 5)
(SEQ ID NO: 12)
FVNQHLC;
and
(f) (Peptide 6)
(SEQ ID NO: 5)
ERGFFYTPKT.

The location of these peptides in the insulin amino acid sequence is shown in FIG. 8.

### Example 3—Antibody Binding to Native and Oxidised Peptides in Sera from Subjects with T1D

A competitive displacement assay was performed to map the exact epitope, namely the exact amino acid sequence binding specificities and to test the binding to native peptides versus oxidised insulin peptides. The binding to the peptide was done by direct ELISA on the native or modified peptides or by competition ELISA. Initially native synthetic peptide was oxidised by exposing to 0.05 mM CuCl₂; 90 mM H₂O₂; 10 mM NaOCl. Native and modified peptides were fractionated by AKTA to evaluated changes in molecular profile. AKTA fractions were then tested by ELISA for reactivity (FIGS. 2 and 3). We next perform competition ELISA with the native peptides and peptides exposed to 0.05 mM CuCl₂; 90 mM H₂O₂; 10 mM NaOCl. Serum samples were pre-incubated with either native insulin peptides or oxidised insulin peptides. Finally, based on the amino acid sequence of the identified peptides we predicted the amino acids that would be oxidised by ^{●}OH and HOCl and designed synthetic peptides that contain the oxidised amino acids in the synthesis. Synthetic peptides were produced by standard commercially available methods, specifically by Peptide Protein Research Ltd. Briefly, peptide synthesis was performed by automated solid phase peptide synthesis (SPPS), which was followed by purification. Each individual amino acid was added to the growing chain according to the sequence. Where specified, dihydroxyphenylalanine was added to replace Tyr or cysteate were added to replace Cys. Similarly, these peptides were tested by competition ELISA. The table below describe the different competition ELISA performed:

Plate coating/
competitor

| | | | |
|---|---|---|---|
| Native insulin | Native peptide | In house oxidised | Synthetic oxidised |
| | | peptide | peptide |
| Oxidised | Native peptide | In house oxidised | Synthetic oxidised |
| insulin | | peptide | peptide |

Serum samples from children with type 1 diabetes were pre-incubated with the synthetic peptides before they were added to the native insulin or oxidised that were previously coated on the ELISA plate. If the antibodies in the serum sample bind to the peptide they will no longer bind to the insulin or oxidised insulin on the plate-competition ELISA. 1:200 dilutions of serum samples from T1D serum samples were pre-incubated for 2 hr at room temperature with the various peptides as shown in the table above. After incubation, serum/peptide mix was added to the ELISA plate previously coated overnight with either native insulin or oxidised insulin for an additional 2 hr incubation. Plates were then washed in a plate washer three times with PBS 0.05% tween. After washing, 100 ul of anti-human IgG diluted 1:1000 in 1% milk-PBS 0.05% tween were added for additional 1 hour and 30 min incubation. After washing, ELISA was developed by adding 100 ul of TMB (10 mg/ml in DMSO) in 10 ml of 0.1 M sodium acetate plus 2 ul of H₂O₂. Reaction was stopped by adding 0.5 M H₂SO₄ (usually 20 min for insulin ELISA).

When type 1 diabetes sera were pre-incubated with an excess amount of native insulin peptide no significant displacement occurred. In contrast, pre-incubation of sera with an excess of oxPTM-INS peptide whether they were oxidised in house or via oxidative synthesis we observed a strong reduction in binding (see FIGS. 2 and 3), indicating that oxidised peptides 6 and 3 are the neoepitope to which auto-antibodies in T1D bind. Oxidised peptide 3 reduced binding to oxidised whole insulin in the range of 40-80% while peptide 6 reduce binding in the range of 35-70%. Nt-INSP-4, however, displayed a comparable inhibition compared to oxPTM-INSP-4 (FIG. 11)

### Example 4—Antibody Binding Combinations of Oxidised Peptides in Sera from Subjects with T1D

When we used a combination of oxidised peptides we did not see a significant increase in blocking the binding to oxidised insulin compared to blocking by either oxidised peptide 3 or oxidised peptide 6 on its own. We used 5 ug peptides and further analysis with reduced peptides need to be performed. We believe that we have used access of peptides in the current experiments and further reduction in the dose of each peptide competitor will give us a better outcome. Furthermore a combination of third competitor peptide will need to be tested.

### Example 5—Autoantibody and T Cell Responses to Oxidative Post-Translationally Modified Insulin Neo-Antigenic Peptides in Type 1 Diabetes

### Methods

Study design. oxPTM-INS was generated in vitro by exposing human recombinant insulin to reactive oxidants. Size exclusion chromatography (ÄKTA) in combination with ELISA was employed to analyse the oxPTM-INS profile and to identify immunogenic fractions resulting from the oxPTM further to LC/MS-MS. Peptides discovered by ÄKTA/ELISA/LC/MS-MS were made and exposed to reactive oxidants, to generate oxPTM-INS peptides (oxPTM-INSP). We also synthesised oxPTM-INSP derivatives designed in silico with oxidised amino acids such as dihydroxyphenylalanine (DOPA) instead of tyrosine, cysteate instead of cysteine, or tyrosine instead of phenylalanine. Autoantibodies to oxPTM-INSPs (in house modified and in silico derivatives) were tested using sera from our biobanks of new onset type 1 diabetes (Study cohort 1). For T-cell stimulation we collected fresh blood samples from type 1 diabetes patients (Study cohort 2) to evaluate, in parallel, CD4⁺ and CD8⁺ T-cells and autoantibody responses to the oxPTM-INSPs (FIG. 14).

### Patient Cohorts.

Study cohort 1: serum sample obtained from the following biobanks: (i) Linköping University (n=50), including sera from young patients at 10 days after type 1 diabetes diagnosis, under insulin therapy for 10 days; (ii) the Immunotherapy of Diabetes (IMDIAB) cohort (n=13) including sera from young subjects with newly diagnosed type 1 diabetes collected before insulin therapy. Thirty age- and sex-comparable non-diabetic subjects were used as controls (Table 1).

Study cohort 2: fresh blood samples collected from 18 subjects with type 1 diabetes: 13 adults with disease duration ≤two years, and five newly-diagnosed children naive to insulin treatment. Eleven non-diabetic subjects were used as controls. Blood samples were collected at Università Campus Bio-Medico (Rome, Italy) and Università Federico II (Naples, Italy) (Table 1).

Study cohort 3: fresh blood samples from five type 1 diabetes adult subjects with disease duration between 2 and 10 years (Table 3).

Type 1 diabetes was diagnosed according to ADA criteria in most cases diagnosis was confirmed by islet-autoantibodies. The ethical committees at Università Campus Bio-Medico, Rome, Italy; Università Federico II, Naples, Italy; Linköping University, Linköping, Sweden and Benaroya Research Institute, Seattle, Washington, US have approved the use of blood samples for research with informed consent signed by the participants or their parents/caregivers.

Insulin modifications. The insulin used for epitope mapping were from two different sources (i) human recombinant insulin from Sigma (product code #I2643) and (ii) human recombinant insulin Humulin R® (Eli Lilly). Sigma insulin was dissolved in PBS (1 mg/ml) while Humulin R® was formulated by the manufacturer at a concentration of 3.47 mg/ml. Insulin was chemically modified as previously described [18] while testing a range of oxidation conditions with NaOCl (HOCl modification, BDH, Oxford, UK) and/or with CuCl₂ (Sigma, Haverhill, UK) plus hydrogen peroxide (^{●}OH modification, Sigma, Haverhill, UK) to further optimise modifications.

ÄKTA pure protein purification. Size exclusion chromatography (ÄKTA purifier system) was used to fractionate the various insulin fragments obtained from oxPTM. Superdex 30 increase column (GE Healthcare) was suitable for the detection of low molecular weights (100-7000 kDa). Chromatographic profiles at the absorbance wavelength of 280 nm were recorded of both native insulin (Nt-INS) and oxPTM-INS.

ELISA for autoantibody detection. The ELISA analysis of Nt-INS and oxPTM-INS autoantibodies was performed as previously described [18](detailed in ESM methods, ELISA assay for antibody detection).

Mass spectrometry. Fractions that showed reactivity in ELISA were dried and resuspended in 30 μl of 0.1% formic acid. Fractions were analysed by LC-MS/MS (Orbitrap Velos, Thermo Scientific) at the Cambridge University UK core facility. The analysis was based on the cleaved peptides following the oxidation producing singly charged ions which are not ordinarily selected for MS/MS in a typical proteomic experiment usually digested with specific enzymes resulting in well-defined peptide cleavage. The breakdown of the insulin was dependent on oxidation whereby cleavage sites are less-well defined and more as a result of random events.

Analysis of insulin synthetic peptides (produced by Peptide Protein Research Ltd, ESM methods, Peptides synthesis-modification-assessment) was done by ultra-performance liquid chromatography coupled with an electrospray ionization quadrupole time-of flight mass spectrometry operating in MSE mode (UPLC-qTof/MS^{e}), which was used to identify all peptides, and to generate fragment ions upon collision induced dissociation (CID) to positively confirm their sequences (ESM methods, Peptide Fractionation and Mass Spectrometry). Structure changes induced by oxPTM were studied by circular dichroism data analysis using the BeStSel server [20] (ESM methods, Structural changes analysis by Circular dichroism).

In vitro peptide stimulation and T-cell proliferation assay. Peripheral blood mononuclear cells (PBMCs) were freshly isolated from type 1 diabetes and healthy individuals using Ficoll-Hypaque density gradient centrifugation. PBMCs were labelled with the fluorescent dye CellTrace Violet (Invitrogen, Thermo Fisher Scientific) and cultured (2×10⁵ cells/well) in round-bottom 96-well plates (Falcon, Becton Dickinson) with RPMI-1640 medium (Gibco, Thermo Fisher Scientific) supplemented with 5% autologous plasma in the presence or not of insulin peptides (20 μg/ml); PPD (10 μg/ml) and anti-CD3 (0.1 μg/ml; clone OKT3) were used as positive control. Two scrambled peptides: DNRDGNVYYF (SEQ ID NO: 17), GRKAETELLVYPTCVYLFFG (SEQ ID NO: 18), and the Exendin 9-39 fragment were used as negative controls. After 7 days, PBMCs were stained with PE-Cy7 anti-CD8 (clone RPA-T8, BD Pharmingen) and FITC anti-CD3 (clone UCHT1, BD Pharmingen). Samples were analysed by using a FACSCanto II (BD Bioscience) to evaluate T-cell proliferation measured as CellTrace Violet dilution. Cytofluorimetric analyses were performed using FlowJo Software (FlowJo, LLC). The results were given as stimulation index (SI), calculated as percentage of stimulated T-cell subset proliferation/percentage of unstimulated T-cell subset proliferation. Assay for detection of peptide specific T cells subsets was done as previously described [37] (detailed in ESM methods, Assay for detection of peptide specific T cells)

Statistical analyses. Statistical analyses were performed using Prism Software 9.0 (GraphPad, San Diego, CA, USA). Cut-off points of positivity (binders) in the antibody ELISA for each peptide were defined by the mean of optical absorbance (O.D) of healthy controls to the corresponding Nt-INSP plus three times the standard error of the mean (SEM). Specificity and sensitivity were evaluated by receiver operating characteristic (ROC) curve analysis. Area under the curve (AUC) is reported as absolute value and was tested for equality according to DeLong et al [21]. Differences in antibody levels and T-cell stimulation indices (SI) between groups were tested by the One way ANOVA or Student's t-tests as appropriate. Correlation analyses were tested by Pearson or Spearman's test, as appropriate. Categorical analyses were performed by Chi-square or McNemar's tests, as appropriate. For each set of experiments, p values were adjusted for multiple comparison using the Holm-Sidak's test. Hierarchical cluster and Principal Component Analysis (PCA) was done using Clustvis software (https://biit.cs.ut.ee/clustvis/) and Prism Software 9.0, respectively.

### Results

Mapping of the oxidized amino acid hotspots in the oxPTM-INS. For the epitope mapping, we used multiple size exclusion chromatography (ÄKTA), ELISA and LC-MS/MS experiments for Sigma insulin and Humulin R® insulin. We first confirmed that reactivity pattern of type 1 diabetes samples to Humulin R® oxPTM-INS was similar to Sigma oxPTM-INS (FIG. 15). Size exclusion chromatography fractions of oxPTM-INS corresponding to small insulin fragments resulting from oxPTM were collected and analysed by ELISA. Fractions that showed reactivity by ELISA were dried and analysed by LC-MS/MS (FIG. 7 and FIG. 16). We have previously reported that amino acids His⁵, Cys⁷, Tyr¹⁶, Phe²⁴ and Tyr²⁶ in the beta-chain are oxidized hotspots. In the current study, additional new oxidized amino acid modification hotspots were discovered: His¹⁰, Leu¹⁷, Cys¹⁹ and Phe²⁵ of the beta-chain and Cys⁶, Cys⁷, Cys¹¹, Tyr¹⁴ and Cys²⁰ of the alpha-chain. Oxidation of Cys⁶ in the alpha-chain was also seen in the Nt-INS (FIG. 8).

LC-MS/MS experiments data mapped neoepitopes to six potential oxPTM-INSP that span both insulin alpha- and beta-chains. Candidate insulin peptides (INSPs) included: SLYQLENYCN (SEQ ID NO: 1) (A:12-21, INSP-3) from the alpha-chain and additional five peptides from the beta-chain: YLVCGERGFF (SEQ ID NO: 10) (B:16-25, INSP-1), LVEALYLVCGER (SEQ ID NO: 11) (B:11-22, INSP-2), LVEALYLVCGERGFFYTPKT (SEQ ID NO: 4) (B:11-30, INSP-4), FVNQHLC (SEQ ID NO: 12) (B:1-7, INSP-5), ERGFFYTPKT (SEQ ID NO: 5) (B:21-30, INSP-6). We also included another version of INSP-6 with the addition of a C-terminal arginine (R) as this sequence was seen in several MS profiles and R is the amino acid in the junction with the proinsulin C-peptide (Table 2).

Antibody reactivity of type 1 diabetes serum against the candidate oxPTM-INSP. Identified peptide candidates were synthesised and exposed to either HOCl or ^{●}OH to generate oxPTM-INSPs that were first assessed by TOF-MS/ES+TIC to confirm modification (FIG. 17). Antibody response against native INSPs (Nt-INSPs) and oxPTM-INSPs was evaluated by ELISA using sera from Study cohort 1. Serum antibody binding experiments revealed the highest number of type 1 diabetes binders (cut-off defined as mean binding of healthy control to Nt-INSP-3 plus three times SEM) for ^{●}OH-modified oxPTM-INSP-3 (86% binders, mean OD=0.667±0.044), HOCl-modified oxPTM-INSP-4 (66% binders, mean OD=0.563±0.053, (cut-off defined as mean binding of healthy control to Nt-INSP-4 plus three times SEM) and ^{●}OH-modified oxPTM-INSP-6 (83% binders, mean OD=0.461±0.013, (cut-off defined as mean binding of healthy control to Nt-INSP-6 plus three times SEM) (FIG. 9a-c, Table 4). No significant reactivity was observed for INSP-1, INSP-2 and INSP-5 (data not shown). For oxPTM-INSP-3 we observed high background binding of healthy controls (p>0.05, FIG. 9a, Table 4). For oxPTM-INSP-4 and oxPTM-INSP-6, binding of type 1 diabetes serum was significantly stronger compared to controls (p=0.0204, p=0.0176 and p=0.0005, for native, ^{●}OH and HOCl oxPTM-INSP-4 and p<0.0001, p<0.0001 and p=0.0187, for native, ^{●}OH and HOCl oxPTM-INSP-6; FIG. 9b-c, Table 4). INSP-6 showed the highest specificity and sensitivity with AUC of 0.879, 0.875 and 0.740 for native, ^{●}OH- and HOCl-modified INSP-6 (Table 4, FIG. 18).

Designing in silico oxPTM-INSPs. We designed in silico multiple oxPTM-INSP derivatives corresponding to one or more aminoacidic modifications. For INSP-3 (SLYQLENYCN (SEQ ID NO: 1)) we synthesised the following oxPTM-INSP-3 derivatives: SL-DOPA-QLENY-Cysteate-N(SEQ ID NO: 2) where tyrosine (Y) was converted to DOPA only in one position and cysteine (C) to Cysteate: SL-DOPA-QLENY-Cysteate-N (SEQ ID NO: 2). An additional oxPTM-INSP-3 was synthesised where both Y residues were converted to DOPA: SL-DOPA-QLEN-DOPA-Cysteate-N(SEQ ID NO: 3). To make the in silico oxPTM-INSP-6 of ERGFFYTPKT (SEQ ID NO: 5), phenylalanine (F) was converted to Y, and Y to DOPA. We thus synthesised two oxPTM-INSP-6 versions of ERGFFYTPKT (SEQ ID NO: 5): ERGYYYTPKT (SEQ ID NO: 6) and ERGYY-DOPA-TPKT (SEQ ID NO: 7). We also included another oxPTM-INSP-6 version with a C-terminal arginine (R), ERGYYYTPKTR(SEQ ID NO: 9), as this sequence was seen in several MS profiles and R is the amino acid in the junction with the proinsulin C-peptide (FIG. 7). Peptide sequence of native and their corresponding in silico oxPTM-peptides were confirmed by UPLC-qTOF/MS (detailed ESM Result section ‘Peptide sequence confirmation by UPLC-qTOF/MS^{e}’, FIG. 19). Structure changes induced by oxPTM were then studied by circular dichroism analysis (ESM results, Structural changes in the oxPTM-INSPs compared to native peptides, FIG. 20)

Antibody reactivity of type 1 diabetes serum against in silico modified oxPTM-INSPs. In type 1 diabetes patients (study cohort 1), we observed a non-significant increase binding to SL-DOPA-QLENY-Cysteate-N(SEQ ID NO: 2) and SL-DOPA-QLEN-DOPA-Cysteate-N(SEQ ID NO: 3) (54% and 57% binders, respectively) compared to SLYQLENYCN (SEQ ID NO: 1) (49% binders, p>0.05); binding of type 1 diabetes samples was, however, significantly more frequent compared to healthy controls (9%, 17% and 30% controls bound to SLYQLENYCN (SEQ ID NO: 1), SL-DOPA-QLENY-Cysteate-N(SEQ ID NO: 2) and SL-DOPA-QLEN-DOPA-Cysteate-N(SEQ ID NO: 3), with p=0.0006, p=0.0112 and p=0.0029 versus type 1 diabetes, respectively) (FIG. 9d, Table 4). There was no increase in specificity/sensitivity in binding to oxPTM-INSP-3 derivatives compared to the Nt-INSP-3 with AUC 0.670, 0.707 and 0.664, for SLYQLENYCN (SEQ ID NO: 1), SL-DOPA-QLENY-Cysteate-N(SEQ ID NO: 2) and SL-DOPA-QLEN-DOPA-Cysteate-N(SEQ ID NO: 3), respectively (Table 4, FIG. 18).

We observed a significant increased binding of type 1 diabetes samples to both ERGYYYTPKT (SEQ ID NO: 6) and ERGYYYTPKTR (SEQ ID NO: 9) with 100% and 88% binders, respectively, compared to 25% and 48% binders in controls, respectively (p23 0.004). In type 1 diabetes patients, binding to oxPTM-INSP-6 derivatives ERGYYYTPKT (SEQ ID NO: 6) or ERGYYYTPKTR (SEQ ID NO: 9) was significantly higher compared to the native ERGFFYTPKT (SEQ ID NO: 5) (p≤0.008). Similarly, a significant increase in binding to ERGYY-DOPA-TPKT (SEQ ID NO: 7) was observed, compared to the native ERGFFYTPKT (SEQ ID NO: 5) (p=0.008, FIG. 9e). We did not observe a significant difference in specificity/sensitivity of Nt-INSP-6 vs. in silico modified oxPTM-INSP-6 derivatives with AUC 0.8686, 0.8542 and 0.8340, respectively (Table 4, FIG. 18).

A competitive displacement assay was performed to evaluate serum binding specificities to oxPTM-INSPs by pre-incubating sera with Nt- or oxPTM-INSPs. Interestingly, oxPTM-INSP-3 and oxPTM-INSP-6, but not Nt-INSP-3 or Nt-INSP-6, were able to inhibit the binding of type 1 diabetes samples to oxPTM-INS (p<0.001), but not to Nt-INS (FIG. 10 a-c; g-i). Competition with combined oxPTM-INSP-3 and oxPTM-INSP-6 did not increase blocking to oxPTM-INS binding compared to a single peptide (data not shown). Nt-INSP-4, however, displayed a comparable inhibition compared to oxPTM-INSP-4 (FIG. 10 d-f.

T-cell stimulation with oxPTM-INSPs. To evaluate the immune cell response against the oxPTM-INSPs, we performed CD4+ and CD8+ T cells proliferation experiments using freshly isolated PBMCs (Study cohort 2, Table 1). Response was calculated as stimulatory index (SI) over unstimulated T-cells.

We found that Nt-INSP-4 (LVEALYLVCGERGFFYTPKT (SEQ ID NO: 4)) induced the strongest stimulation in type 1 diabetes compared to controls for both CD4⁺ (mean SI: 119.8±51.69 vs. 6.89±3.4, p<0.001; FIG. 11a) and CD8⁺ T-cells (mean SI: 405.8±325.5 vs. 5.948±3.125, p=0.049; FIG. 11c). Of note, as highlighted by the heatmaps in FIGS. 11b and 4d, heterogeneous response also to other peptides was evident across different type 1 diabetes individuals, with some patients preferentially responding to various derivatives of oxPTM-INSP-3 (SL-DOPA-QLENY-Cysteate-N(SEQ ID NO: 2), SL-DOPA-QLEN-DOPA-Cysteate-N(SEQ ID NO: 3)) and oxPTM-INSP-6 (ERGYYYTPKT (SEQ ID NO: 6), ERGYY-DOPA-TPKT (SEQ ID NO: 7), ERGYYYTPKTR (SEQ ID NO: 9)). To better assess specificity of T-cell stimulation in type 1 diabetes compared to controls, we analysed response according to different SI cut-offs. When using a SI>3, we found a larger number of type 1 diabetes subjects with a CD4⁺ response to oxPTM-INSP-6 derivatives compared to controls (66.7% vs. 27.3%; p=0.039), while response to Nt-INSP-4 and oxPTM-INSP-3 was similar between patients and controls (Nt-INSP-4: 66.7% vs. 45.5%; oxPTM-INSP-3 22.2% vs. 9.1%) (Table 5). When comparing response to oxPTM-INSPs and Nt-INSPs among type 1 diabetes patients, we found that CD4⁺ response to oxPTM-INSP-6 was more frequent compared to Nt-INSP-6 (66.7% vs 27.8%; p=0.045) (FIG. 11 a-b, Table 5). CD8⁺ T-cells responses to the tested peptides were also common in type 1 diabetes subjects, who responded with similar frequency to oxPTM-INSP-6 and Nt-INSP-4 (72.2% patients showed a SI>1 for both); such response was higher in type 1 diabetes compared to controls for oxPTM-INSP-6 (72.2% vs. 27.3%; p=0.02), but not for Nt-INSP-4 (72.2% vs. 63.6%; p=NS) (Table 6). Higher SI cut-offs did not reveal significant differences between groups (Table 6).

Correlation analysis showed association between T-cell responses to the oxPTM-INS-6 derivative ERGYYYTPKTR (SEQ ID NO: 9) (but not Nt-INSP-6) and Nt-INSP-4, for both CD4⁺ (r=0.59, p=0.12; FIG. 5a) and CD8⁺ (r=0.83, p=0.002; FIG. 12b). The CD4⁺ T cell response to Nt-INSP-4 was also strongly correlated to the CD8⁺ T cell response of oxPTM-INSP derivatives ERGYYYTPKTR (SEQ ID NO: 9) and SL-DOPA-QLENY-Cysteate-N(SEQ ID NO: 2) (r≤0.83, p≤0.002), but not their native counterparts (FIG. 12c), suggesting an overlap in CD4⁺ and CD8⁺ T-cell responses involving Nt-INSP-4, oxPTM-INSP-3 and oxPTM-INSP-6.

We next utilized surface staining for CD45RA and CCR7 on CD154+CD69+ T-cells to classify epitope specific T-cells as Naïve (CD45RA+CCR7+), central memory (TCM, CD45RA−CCR7+), effector memory (TEM, CD45RA−CCR7−) or effector memory cells re-expressing CD45RA (TEMRA, CD45RA+CCR7−). Across five representative subjects with established type 1 diabetes (Cohort 3, Table 3) we detected TCM, TEM and TEMRA, with naïve cells also present. Nt-INS and oxPTM-INS specific T-cells had a higher percentage of naïve cells than the influenza control (44.7% and 41.1%, respectively) but appreciable percentage of TCM and TEM were also present, suggesting that there is an existing pool of memory T-cells that recognized these insulin peptides in subjects with type 1 diabetes (FIG. 21-22).

Correlation between T-cell stimulation and antibody response. Subjects evaluated for T-cell stimulation were also tested for antibody reactivity to either oxPTM-INSPs or oxidised intact insulin (oxPTM-INS) to assess correlations between humoral and cellular responses (FIG. 11-e-f). In study cohort 2, antibody reactivity to oxPTM-INSP-6 was the highest as observed in the study cohort 1, with 11/18 (61.1%) binding to at least one oxPTM-INSP-6 derivatives (p<0.001 oxPTM-INSP-6 vs. Nt-INSP-6). Detailed analysis of autoantibody response in this cohort is described in the ESM (ESM results Antibody binding to oxPTM-INSPs in Study cohort 2).

We then analysed the extent of correlation between CD4⁺, CD8⁺ and IgG antibody responses. CD4⁺ and CD8⁺ responses to oxPTM-INSP-3 overlapped in 9/18 (50.0%), but only 1/18 patients (5.5%) showed concordant antibody reactivity (FIG. 13a). The CD4⁺ T cell response to Nt-INSP-4 frequently overlapped with CD8⁺ (13/18 [72.2%]), and to a lesser extent to antibodies (7/18 [38.8%]). Overall, 4/18 (22.2%) patients had a concordant CD4⁺, CD8⁺ and antibody response to Nt-INSP-4 (FIG. 13b). CD4⁺ response to oxPTM-INSP-6 was linked to both CD8⁺ and/or antibodies: 12/18 (72.2%) patients had concordant CD4⁺ and CD8⁺ responses, while 9/18 (50%) patients had concordant CD4⁺ and antibody responses. Overall, 8/18 (44.4%) patients showed an immune response involving simultaneously CD4⁺, CD8⁺ and antibodies (FIG. 13c). CD4⁺ T-cell stimulation to Nt-INSP-4, oxPTM-INSP-6 and oxPTM-INSP-3 was associated with antibody reactivity to oxPTM-INS in 8/18 (44.4%), 8/18 (44.4%) and 7/18 (38.9%) type 1 diabetes patients. Concordant autoimmune response to oxPTM-INSP involving simultaneously CD4⁺, CD8⁺ T-cells and autoantibodies to oxPTM-INS was seen in 5/18 (27.8%), 6/18 (33.3%), and 4/18 (22.2%) type 1 diabetes patients for Nt-INSP-4, oxPTM-INSP-6 and oxPTM-INSP-3, respectively (FIG. 13d-f), suggesting that CD4⁺ T-cell response to these peptides is required to generate CD8⁺ and/or antibody responses to oxPTM-INS.

We next performed hierarchical cluster analysis (Euclidean distance, Ward's method) of patients, and of peptides. Hierarchical cluster analysis and principal component analysis (PCA) revealed association between the responses to different oxPTM-INSP and identify clustering of type 1 diabetes versus healthy control samples. We observed association between Nt-INSP-4 and ox-PTM-INS-P6, ERGYYYTPKTR (SEQ ID NO: 9) for CD4⁺ and CD8⁺. For IgG response ERGYY-DOPA-TPKT (SEQ ID NO: 7) is associated with ^{●}OH-Insulin. We also observed clustering of response of type 1 diabetes samples using PCA analysis of all responses, CD4, CD8 and IgG. We observed cluster of 11 type 1 diabetes samples with PC1>0 while the rest clustered with healthy control samples with PC1<0 (FIG. 23).

### SUMMARY

In this study, we show that neo-antigenic insulin peptides generated by oxPTM are targeted by both circulating autoantibodies and T-cells in patients with type 1 diabetes. The main autoimmune response involved three insulin peptides: B:11-30, B:21-30, A:12-21 and, their respective oxPTM-INSP derivatives: oxPTM-INSP-4 (B:11-30), oxPTM-INSP-6 (B:21-30) and oxPTM-INSP-3 (A:12-21).

We identified multiple cleavage sites after exposure of insulin to oxidants (^{●}OH and HOCl). Consistent with literature, cleavage resulting from oxidative damage occur preferentially between the residues phenylalanine, cysteine, glycine, leucine, valine, and tyrosine, as well as near the cysteine bridges, especially in the alpha-chain [23]. As previously described [22], we observed structural changes within insulin derived peptides oxPTM-INSP-3 and oxPTM-INSP-6 as a results of oxidations.

It appears that the nature of the modification itself provides new interaction properties (e.g. additional hydrogen bonding potential), or opens the access to hidden epitopes that could contribute to the formation of immunogenic products. Peptide cleavage makes self-antigens more accessible to the immune system and represents a step required for antigen presentation. It has been shown that B:21-29 is a CD8 epitope generated by proteasome cleavage during antigen presentation [22]. Our data suggest that a similar epitope (B:21-30) can result from beta-chain cleavage by oxidation. We speculate that oxidative cleavage may facilitate antigen presentation via a proteasome independent pathway by providing readily accessible peptides to the immune system.

We found antibodies to oxPTM-INSP-6 in most individuals with type 1 diabetes. Of note, we observed the same pattern of response for oxPTM-INSP-6 that was oxidised in house compared to in silico designed derivatives (ERGYYYTPKT (SEQ ID NO: 6) and ERGYY-DOPA-TPKT (SEQ ID NO: 7)), suggesting that oxidation of F to Y and Y to DOPA generates neoepitopes that are recognised by specific antibodies. Interestingly, methyldopa (an analogue of DOPA) can block the activation of insulin autoreactive T-cells in NOD mice and prevented beta cell loss and IAA in recent onset type 1 diabetes [24]. In contrast to oxPTM-INSP-6, oxPTM-INSP-3 was less specific revealing increased background in control subjects. This can be due to the spontaneous oxidation of cysteine or to the presence of a free thiol, which could result in non-specific interaction of SLYQLENYCN (SEQ ID NO: 1), SL-DOPA-QLENY-Cysteate-N(SEQ ID NO: 2) and SL-DOPA-QLEN-DOPA-Cysteate-N(SEQ ID NO: 3). Further chemistry studies will need to address this point in future work. Data from oxPTM-INSP-4 did not clearly substantiate the importance of oxPTM for antibody response to INSP-4. Indeed, Nt-INSP-4 blocked type 1 diabetes serum binding to oxPTM-INS like oxPTM-INSP-4. A similar result was observed for the T-cells, as stimulation with Nt-INSP-4 was stronger compared to other oxPTM-INSPs.

Antibodies to oxPTM-INSP-6 coincided with cellular responses in most cases, implying that antibody reactivity to oxPTM-INS is dependent of CD4⁺ T-cell activation and often associates with CD8⁺ response. Antibody reactivity to oxPTM-INSP-6 (B:21-30) and Nt-INSP-4 (B:11-30) strongly correlated with antibodies to oxPTM-INS. Furthermore, immune responses to oxPTM-INSP-6 and Nt-INSP-4 often coexisted within patients. Sequence homology between the two peptides cannot fully explain the overlap in immune response, because the association was specific to oxPTM-INSP-6 rather than Nt-INSP-6. It is possible that, when modified, oxPTM-INSP-6 gains a structural conformation similar to the C-terminal part of the longer Nt-INSP-4. A second possibility is that the B:11-30 peptide is autoxidised during the experimental procedures. We were not able to systematically design/analyse in silico derivatives of INSP-4. Within the INSP-4 sequence LVEALYLVCGERGFFYTPKT (SEQ ID NO: 4) there are at least 5 oxidatively modifiable amino acid residues corresponding to dozens of potential combinations, in which various numbers of the cysteine, tyrosines and phenylalanines are either oxidised or not oxidised at various locations within the peptide. Thus, we had to restrict our in silico oxidised peptides analysis to the shorter INSP-6 ERGFFYTPKT (SEQ ID NO: 5) and INSP-2 LVEALYLVCGER (SEQ ID NO: 11) containing lower numbers of oxidation-susceptible amino acid residues. No reactivity was observed against LVEALYLVCG (SEQ ID NO: 19). Previously, it has been shown that simple exposure to ambient air can induce oxidation of the insulin peptide B:9-23 [25], which is targeted by γδT-cells in the NOD mouse. Intermolecular epitope spreading, involving native and oxPTM-INS and their derived peptides (Nt-INSP-4 and oxPTM-INSP-6), is another potential mechanism. Taken together, these findings suggest that Nt-INSP-4 and oxPTM-INSP-6 peptides are potential T-cell and antibody neoepitopes in type 1 diabetes. We performed a pilot study to identify the T-cell subsets that are stimulated by oxPTM-INSPs finding an existing pool of memory T-cells that recognize oxPTM-INSPs in subjects with type 1 diabetes. Further studies with a larger sample size will be needed to confirm this observation.

In conclusion, our findings support the concept that oxidative stress, and neo-antigenic epitopes generated by oxPTM of beta cell antigens such as insulin, may involve in the pathogenesis of type 1 diabetes.

TABLE 1
Clinical and biochemical features of the study populations.
Study cohort 1

| | Type 1 diabetes | Healthy controls |
|---|---|---|
| | (N = 63) | (N = 30) |
| Age, years | 11 ± 4.9 | 13.8 ± 0.69 |
| Males, n (%) | 33 (52.4%) | 10 (33.3%) |
| C-peptide, nmol/l | 0.132 ± 0.129 | NA |
| GADA+, n/n (%) | 33/42 (78.5%) | NA |
| IA-2A+, n/n (%) | 35/43 (81.4%) | NA |

Study cohort 2

| | Type 1 diabetes | Healthy controls |
|---|---|---|
| | (N = 18) | (N = 11) |
| Age, years* | 18.75 ± 12.48 | 29.7 ± 8.59 |
| Males, n (%) | 8 (44.4%) | 3 (27.3%) |
| BMI, Kg/m² | 20.00 ± 4.29 | 22.00 ± 5.31 |
| Children, n (%) | 5 (27.7%) | 2 (18.2%) |
| Adults, n (%) | 14 (77.7%) | 9 |
| Insulin-naïve, n (%) | 5 (27.7%) | NA |
| Disease duration, | 1.07 ± 3.08 | NA |
| years | | |
| New-onset, <1 month, | 5 (27.7%) | NA |
| n (%) | | |
| Duration >1 month | 13 (68.4%) | NA |
| and <2 years, n (%) | | |
| HbA1c, % | 8.94 ± 2.23 | NA |
| HbA1c, mmol/mol | 74.2 ± 0.87 | |
| C-peptide (nmol/l) | 0.21 ± 0.13 | NA |

Table shows features related to serum samples of i) Study cohort 1 used for the neo-antigenic peptide discovery experiments, collected at the Linkoping and IMDIAB-Rome biobanks and ii) Study cohort 2 tested in the T cell stimulation experiments, newly recruited in Rome, Università Campus Bio-Medico, and Naples, Università Federico II.
Categorical analyses were performed by Chi-square test.
*p = 0.0498

TABLE 2
Six oxidative insulin neoantigenic peptides. List of insulin
peptide (INSP) candidates that were detected by LC-MS/MS following
insulin oxPTM. The table highlights the aminoacidic modifications
detected by LC-MS/MS that are graphically represented in FIG. 8.

| Insulin peptide | | | Oxidised amino |
|---|---|---|---|
| (INSP) | Sequence and SEQ ID NO | | acid hotspots |
| INSP-1 (Peptide 1) | B:16-25 | YLVCGERGFF | Y¹⁶; L¹⁷; C¹⁹; |
| | | (SEQ ID NO: 10) | F²⁴; F²⁵ |
| INSP-2 (Peptide 2) | B:11-22 | LVEALYLVCGER | Y¹⁶; L¹⁷; C¹⁹ |
| | | (SEQ ID NO: 11) | |
| INSP-3 (Peptide 3) | A:12-21 | SLYQLENYCN | Y¹⁴; C²⁰ |
| | | (SEQ ID NO: 1) | |
| INSP-4 (Peptide 4) | B:11-30 | LVEALYLVCGERGFFYTPKT | Y¹⁶; L¹⁷; C¹⁹; |
| | | (SEQ ID NO: 4) | F²⁴; F²⁵; Y²⁶ |
| INSP-5 (Peptide 5) | B:1-7 | FVNQHLC | H⁵; C⁷ |
| | | (SEQ ID NO: 12) | |
| INSP-6 (Peptide 6) | B:21-30 | ERGFFYTPKT | F²⁴; F²⁵; Y²⁶ |
| | | (SEQ ID NO: 5) | |
| INSP-6 + R | B:21-30 | ERGFFYTPKTR | F²⁴; F²⁵; Y²⁶ |
| (Peptide 6 + R) | | (SEQ ID NO: 8) | |

### Electronic Supplementary Material (ESM)

ESM Methods ELISA assay for antibody detection. The ELISA analysis of native (Nt-INS) and modified insulin (oxPTM-INS) was performed as previously described [18-19]. Briefly, the ELISA plate was coated overnight at 4° C. with 100 μl per well of modified or native insulin in 0.05 M carbonate/bicarbonate buffer pH 9.6 at 10 μg/ml. The next day, the plates were washed 3 times with 0.1% Tween PBS, followed by blocking for 2 hours at room temperature with 200 μl per well of 5% BSA in 0.1% Tween PBS. After washing, 100 μl of 1:200-diluted serum samples in 5% BSA-0.1% Tween PBS were added for additional 2 hours incubation. ELISA plates were then washed 3 times with 0.1% Tween PBS followed by probing with anti-human IgG horse radish peroxidase conjugated (HRP, Sigma) at 1:1,000 dilution in 5% BSA-0.1% Tween PBS for another 1.5 hours incubation. The ELISA plates were washed, and 100 μg/ml TMB substrate in 0.1M sodium acetate pH 6.0 plus 2 μl/10 ml of H₂O₂, were added. Subsequently, the reaction was stopped with 20% v/v sulphuric acid. The optical density (O.D.) was measured at 450 nm using a GENios plate reader and Magellan software (TECAN, Dorset, UK).

Binding of serum to for oxPTM-INS fragments was also done by ELISA. Fraction from the AKTA runs were collected further to 1:10 dilution in PBS before coating microtiter plates for further overnight incubation at 37° C. which followed the assay as above.

To assess the binding specificity of serum samples to identified peptides, a competitive ELISA was performed. The ELISA was carried out following the method described above, except that the serum samples were pre-incubated for 2 h with and without 10 μg/ml peptides as competitors, before adding the serum samples to the insulin coated ELISA plate.

Peptides synthesis-modification-assessment. The peptides were synthesized using standard Fmoc solid phase chemistry (Shepherd and Atherton). Peptides were cleaved from the appropriate resin using TFA containing silanes and ethane dithiol, which removed all side chain protecting groups. If the acetonide protecting group had not been fully cleaved using this methodology the peptide was dissolved in an aqueous buffer containing 0.1% TFA and the peptide monitored by MS until complete removal of the group was verified. The peptides were then purified using HPLC on a C18 column (Phenomenex) using a gradient elution profile of water:acetonitrile and 0.1% TFA. Peptides containing cysteate were synthesised using Fmoc-L-Cysteic acid (Carbosynth) and those containing dihydroxyphenylalanine (DOPA) were synthesised using Fmoc-DOPA (acetonide)-OH (Carbosynth). These two amino acids were treated the same as standard Fmoc amino acids.

Peptide Fractionation and Mass Spectrometry. Ultra-performance liquid chromatography coupled with an electrospray ionization quadrupole time-of flight mass spectrometry operating in MSE mode (UPLC-qTof/MS^{e}) was used to identify all peptides, and to generate fragment ions upon collision induced dissociation (CID) to positively confirm their sequences. The analysis was executed on an ACQUITY H-Class UPLC system (Waters, Milford, USA) coupled to a qTOF High Definition Mass Spectrometer (HDMS) Synapt G2Si, equipped with an electrospray ionisation (ESI) interface (Waters, Milford, USA). All six peptide samples were separated chromatographically using a Waters Acquity UPLC BEH C18 column (1.7 μm, 2.1 mm×50 mm). The mobile phases consisted of A (LCMS grade Water, 0.1% formic acid) and B (LCMS grade Acetonitrile, 0.1% formic acid), with the following gradient: 0-2 min, 5% B; 2.0-3.0 min, 5%-45% B; 3.0-3.1 min, 45%-90% B; 3.1-4.0 min, 90% B; 4.0-4.1 min, 90%-5% B, 4.1%-5% B. The flow rate was set at 0.45 mL/min. The temperature in the auto sampler and in the column oven was set at 10° C. and 60° C., respectively. MS data were collected from m/z 100-1500 Da in positive MSE continuum mode. The electrospray ionization conditions were set as follows: capillary voltage, 3.5 kV; cone voltage, 40 V; cone gas flow, 50 L/h; source temperature, 120° C.; desolvation gas flow, 800 L/h; and desolvation temperature, 450° C. Collision energy was set at 4 V in low energy acquisition; whereas high energy collision energy ramp wat set at 10-40 V. Leucine Encephalin (m/z 556.2771) was used for lock mass at a concentration of 200 μg/mL and a flow rate of 20 μl/min. Data acquisition was carried out with Masslynx v4.2 (Waters, Milford, USA), whereas data were processed using the UNIFI Scientific Information System v1.8 software (Waters, Milford, USA).

Structural changes analysis by Circular dichroism. Structural changes were determined by Circular dichroism. Nt-INSP-6 and oxPTM-INSP-6 were resuspended in PBS buffer while Nt-INSP-3 was resuspended in 50% (v/v) methanol, 50% (v/v) PBS buffer, oxPTM-INSP-3 Ac-SL-DOPA-QLENY-Cysteate-N (SEQ ID NO: 15) peptide was resuspended in 25% (v/v) methanol, 75% (v/v) PBS buffer and oxPTM-INSP-3 Ac-SL-DOPA-QLEN-DOPA-Cysteate-N(SEQ ID NO: 16) peptide was resuspended in 15% (v/v) methanol, 85% (v/v) PBS buffer. Far-UV CD spectra were measured in a Chirascan (Applied Photophysics) spectropolarimeter thermostated at 20° C. The spectra of peptides (0.05 mg/ml) were recorded from 260 to 200 nm, at 0.5 nm intervals, 1 nm bandwidth, and a scan speed of 10 nm/min. Three accumulations were averaged for each spectrum, Data analysis was carried out using the BeStSel server [20].

Assay for detection of peptide specific T cells. An activation induced marker (AIM) to label and characterize native (Nt-INS) and modified insulin (oxPTM-INS) responsive T cells and influenza specific T cells (as a positive control) was performed as previously described [37]. Briefly, PBMC from subjects with type 1 diabetes were plated at a density of 10 million cells per ml in 24 well plates and pulsed with solvent only (Mock) or specific peptides of interest (oxPTM-INSP, Nt-INSP, or an influenza peptide) in individual wells for 10-14 hours in the presence of an anti-CD40 blocking antibody (Miltenyi Biotec). Activated T cells were labelled with anti-CD154 APC (Miltenyi Biotec) followed by anti-APC beads (Miltenyi Biotec) and magnetically enriched using columns (Miltenyi Biotec), reserving a 1% fraction of the non-enriched cells to determine the total number of T cells in the sample. The enriched and reserved cells were labelled with anti-CD4 BUV395 (BD Biosciences), anti-CD69 PE-Cy7 (BioLegend), anti-CD45RA AF700 (BD Biosciences), anti-CCR7 APC-Cy7 (BioLegend), plus anti-CD14 PerCP-Cy5.5 (BD Biosciences) anti-CD19 PerCP-Cy5.5 (BD Biosciences) and Viaprobe (BD Biosciences) as cell exclusion markers acquired using an BD LSR II cytometer and analyzed using FlowJo software (Tree Star).

### ESM Results

Peptide sequence confirmation by UPLC-qTOF/MS^{e}. FIG. 19 shows the fragmentation spectra of the three versions of synthetically oxidative modified peptides 3 (oxPTM-INSP-3) and 6 (oxPTM-INSP-6). Native and oxPTM-INSP-3 correspond to common sequence backbone of SLYQLENYCN (SEQ ID NO: 1), [R—NH3]⁺, m/z 1288.551); Ac-SL-DOPA-QLENY-cysteate-N(SEQ ID NO: 15), [R—NH3]⁺, m/z 1352.5162) and Ac-SL-DOPA-QLEN-DOPA-cysteate-N(SEQ ID NO: 16), [R—NH3]⁺, m/z 1368.5091). Fragmentation pattern of oxPTM-INSP-6 versions, ERGFFYTPKT (SEQ ID NO: 5)-NH2, [R—NH3]⁺, m/z 1286.6626; ERGYYYTPKT (SEQ ID NO: 6), [R—NH3]+, m/z 1277.6253 and ERGYY-DOPA-TPKT (SEQ ID NO: 7), [R—NH3]+, m/z 1293.6213 is also shown. Table in FIG. 19 highlights the fragment ions identified with different m/z values as a result of modifications. Detailed CID spectra with 67%-83% matched first generation primary ions within a filtered upper limit of error of measurement of 30 ppm of peptide y and b assigned fragment ions positively identified against their respective putative sequences and expected modifications was collected (not shown).

Structural changes in the oxPTM-INSPs compared to native peptides. Within the context of intact insulin, residues within native peptide 3 form a helix (FIG. 20a). However, the free peptide is significantly unstructured (52%) but with sheet forming propensity (27%) and minor helical conformations (6%). After oxPTM modification, the SL-DOPA-QLENY-cysteate-N(SEQ ID NO: 2) peptide became more structured with increased helicity (16%). However, the SL-DOPA-QLEN-DOPA-cysteate-N(SEQ ID NO: 3) peptide was more disordered (60%) with complete loss of helical structure. Residues corresponding to native peptide 6 are extended within intact insulin and the free peptide (ERGFFYTPKT (SEQ ID NO: 5)) showed both coil (54%) and sheet (29%) conformations. After oxPTM modification ERGYYYTPKT (SEQ ID NO: 6), ERGYY-DOPA-TPKT (SEQ ID NO: 7) and ERGYYYTPKTR (SEQ ID NO: 9) became significantly more sheet-like in structure (10 to 14% increase), while addition of arginine at the C-terminus in peptide Ac-ERGFFYTPKTR (SEQ ID NO: 21) had no effect.

Antibody binding to oxPTM-INSPs in Study cohort 2. When analysing the IgG autoantibody reactivity in Study cohort 2, we found increased binding to NT-INS, ^{●}OH-INS, and insulin peptides compared to healthy controls (p<0.001), with 6/18 (33.3%), 9/18 (50%), and 11/18 (61.1%) type 1 diabetes binders to Nt-INS, ^{●}OH-INS, and insulin peptides, respectively. Of note, 11/18 (61.1%) bound to at least one oxPTM-INSP-6 derivatives, with ERGYY-Dopa-TPKT (SEQ ID NO: 7) being the most reactive peptide (10/18 [55.5%]), while none bound to the Nt-INSP-6 (p<0.001); among type 1 diabetes binders to oxPTM-INSP-6, two patients also showed reactivity to Nt-INSP-3 and oxPTM-INSP-3 (SL-Dopa-QLENY-Cysteate-N(SEQ ID NO: 2), respectively, while 7/18 (38.9%) type 1 diabetes patients showed reactivity to the Nt-INSP-4 peptide. Reactivity to the oxPTM-INSP-6 derivative ERGYY-dopa-TPKT (SEQ ID NO: 7), but not the Nt-INSP-6, displayed a positive correlation with Nt-INSP-4 (LVEALYLVCGERGFFYTPKT (SEQ ID NO: 4)) (r=0.92; p<0.001). Antibody reactivity to both ERGYY-DOPA-TPKT (SEQ ID NO: 7) and LVEALYLVCGERGFFYTPKT (SEQ ID NO: 4) correlated with ^{●}OH-INS (r=0.75, p<0.001; and r=0.76, p<0.001). By contrast, neither Nt-INSP-6 or Nt-INSP-3 correlated with ^{●}OH-INS antibody reactivity.

TABLE 3
Clinical features of subjects with established type 1
diabetes (T1D) from the Benaroya Research Institute.

| | Age at Draw | | Disease duration |
|---|---|---|---|
| Patient | (years) | Gender | (years) |
| T1D #1 | 29 | male | 10.0 |
| T1D #2 | 37 | male | 7.1 |
| T1D #3 | 39 | male | 2.1 |
| T1D #4 | 33 | male | 8.0 |
| T1D #5 | 32 | male | 7.1 |

TABLE 4
Statistical analysis of binding of the various native and oxPTM-insulin
peptides (INSP). The most significant binding observed is for insulin
peptide 6 (INSP-6) that demonstrate the highest specificity with ROC-AUC > 0.8
and 100% binding to the in silico modified oxPTM-INSP-6 (ERGYYYTPKT
(SEQ ID NO: 6) and ERGYY-DOPA-TPKT (SEQ ID NO: 7)). Cut-off points of
positivity (binders) for each peptide were defined by the mean absorbance
of healthy controls to the corresponding Nt-INSP plus three times the
standard error of the mean (SEM). All analyses were corrected for multiple
comparisons using Holm Sidak's test.

| Type 1 diabetes | | Healthy controls | | | |
|---|---|---|---|---|---|
| | % | | % | | p |
| Mean ± SEM | binders | Mean ± SEM | binders | AUC | value |

In house modifications

| | | | | | | | |
|---|---|---|---|---|---|---|---|
| INSP-3 | Native | 0.405 ± 0.029 | 42 | 0.333 ± 0.030 | 19 | 0.648 | 0.4744 |
| | oxPTM-•OH | 0.667 ± 0.044 | 86 | 0.617 ± 0.042 | 86 | 0.515 | 0.6651 |
| | oxPTM-HOCI | 0.473 ± 0.037 | 53 | 0.452 ± 0.048 | 40 | 0.543 | 0.7259 |
| INSP-4 | Native | 0.469 ± 0.047 | 50 | 0.306 ± 0.032 | 20 | 0.730 | 0.0204 |
| | oxPTM-•OH | 0.539 ± 0.068 | 58 | 0.344 ± 0.022 | 27 | 0.705 | 0.0176 |
| | oxPTM-HOCI | 0.563 ± 0.053 | 66 | 0.296 ± 0.022 | 27 | 0.818 | 0.0005 |
| INSP-6 | Native | 0.435 ± 0.014 | 66 | 0.263 ± 0.023 | 10 | 0.879 | <0.0001 |
| | oxPTM-•OH | 0.461 ± 0.013 | 83 | 0.280 ± 0.020 | 12 | 0.875 | <0.0001 |
| | oxPTM-HOCI | 0.391 ± 0.013 | 43 | 0.312 ± 0.019 | 25 | 0.740 | 0.0187 |

In silico modifications

| | | | | | | | |
|---|---|---|---|---|---|---|---|
| INSP-3 | SLYQLENYCN | 0.242 ± 0.019 | 49 | 0.143 ± 0.007 | 9 | 0.670 | 0.0006 |
| | (SEQ ID NO: 1) | | | | | | |
| | SL-DOPA-QLENY- | 0.228 ± 0.016 | 54 | 0.154 ± 0.006 | 17 | 0.707 | 0.0112 |
| | Cysteate-N | | | | | | |
| | (SEQ ID NO: 2) | | | | | | |
| | SL-DOPA-QLEN- | 0.256 ± 0.018 | 57 | 0.169 ± 0.008 | 30 | 0.664 | 0.0029 |
| | DOPA-Cysteate-N | | | | | | |
| | (SEQ ID NO: 3) | | | | | | |
| INSP-6 | ERGFFYTPKT | 0.423 ± 0.012 | 63 | 0.263 ± 0.020 | 16 | 0.868 | <0.0001 |
| | (SEQ ID NO: 5) | | | | | | |
| | ERGYYYTPKT | 0.556 ± 0.031 | 100 | 0.351 ± 0.044 | 25 | 0.854 | 0.004 |
| | (SEQ ID NO: 6) | | | | | | |
| | ERGYY-DOPA-TPKT | 0.536 ± 0.021 | 100 | 0.357 ± 0.030 | 30 | 0.834 | 0.0006 |
| | (SEQ ID NO: 7) | | | | | | |
| | ERGYYYTPKTR | 0.697 ± 0.058 | 88 | 0.423 ± 0.039 | 48 | 0.797 | <0.0001 |
| | (SEQ ID NO: 9) | | | | | | |

TABLE 5
CD4⁺ T-cell response in subjects with type 1 diabetes and
controls according to different stimulatory index (SI) cut-offs.

| Type 1 diabetes (N = 18) | | | | Controls (N = 11) | | | |
|---|---|---|---|---|---|---|---|
| SI > 1 | SI > 3 | SI > 5 | SI > 10 | SI > 1 | SI > 3 | SI > 5 | SI > 10 |

Nt-INSP-3

| | | | | | | | | |
|---|---|---|---|---|---|---|---|---|
| SLYQLENYVN | 13 | 5 | 4 | 3 | 6 | 2 | 1 | 0 |
| (SEQ ID NO: 1) | (72.2%) | (27.8%) | (21.1%) | (16.7%) | (54.5%) | (18.2%) | (9.1%) | (0.0%) |

oxPTM-INSP-3 derivatives

| | | | | | | | | |
|---|---|---|---|---|---|---|---|---|
| SL-Dopa-QLENY- | 9 | 3 | 2 | 1 | 6 | 2 | 2 | 0 |
| Cysteate-N | (50.0%) | (16.7%) | (10.5%) | (5.6%) | (54.5%) | (18.2%) | (18.2%) | (0.0%) |
| (SEQ ID NO: 2) | | | | | | | | |
| SL-Dopa-QLEN-Dopa- | 12 | 1 | 0 | 0 | 3 | 0 | 0 | 0 |
| Cysteate-N | (66.7%) | (5.6%) | (0.0%) | (0.0%) | (27.3%) | (0.0%) | (0.0%) | (0.0%) |
| (SEQ ID NO: 3) | | | | | | | | |
| at least one oxPTM- | 15 | 4 | 4 | 1 | 8 | 1 | 2 | 0 |
| INSP-3 derivative | (83.3%) | (22.2%) | (21.1%) | (5.6%) | (72.2%) | (9.1%) | (18.2%) | (0.0%) |

Nt-INSP-4

| | | | | | | | | |
|---|---|---|---|---|---|---|---|---|
| LVEALYLVCGERGFFYTPKT | 18 | 12 | 10 | 9 | 9 | 5 | 3 | 1 |
| (SEQ ID NO: 4) | (100%) | (66.7%) | (5.3%) | (50.0%) | (81.8%) | (45.5%) | (27.3%) | (9.1%) |

Nt-INSP-6

| | | | | | | | | |
|---|---|---|---|---|---|---|---|---|
| ERGFFYTPKTR | 10 | 5 | 4 | 2 | 4 | 0 | 0 | 0 |
| (SEQ ID NO: 8) | (55.6%) | (27.8%) | (21.1%) | (11.1%) | (36.4%) | (0.0%) | (0.0%) | (0.0%) |

oxPTM-INS-6 derivatives

| | | | | | | | | |
|---|---|---|---|---|---|---|---|---|
| ERGYY-Dopa-TPKT | 9 | 1 | 1 | 1 | 3 | 0 | 0 | 0 |
| (SEQ ID NO: 7) | (50.0%) | (5.6%) | (5.2%) | (5.6%) | (27.3%) | (0.0%) | (0.0%) | (0.0%) |
| ERGYYYTPKT | 9 | 4 | 0 | 0 | 4 | 2 | 1 | 0 |
| (SEQ ID NO: 6) | (50.0%) | (22.2%) | (0.0%) | (0.0%) | (36.4%) | (18.2%) | (9.1%) | (0.0%) |
| ERGYYYTPKTR | 13 | 8 | 3 | 3 | 7 | 3 | 0 | 0 |
| (SEQ ID NO: 9) | (72.2%) | (38.9%) | (15.8%) | (16.7%) | (63.6%) | (27.3%) | (0.0%) | (0.0%) |
| at least one | 15 | 12 | 4 | 4 | 7 | 3 | 1 | 0 |
| oxPTM-INSP-6 | (83.3%) | (66.7%) | (21.1%) | (22.2%) | (63.6%) | (27.3%) | (9.1%) | (0.0%) |
| derivative | | | | | | | | |

TABLE 6
CD8⁺ T-cell response in subjects with type 1 diabetes and
controls according to different stimulatory index (SI) cut-offs.

| Type 1 diabetes (N = 18) | | | | Controls (N = 11) | | | |
|---|---|---|---|---|---|---|---|
| SI > 1 | SI > 3 | SI > 5 | SI > 10 | SI > 1 | SI > 3 | SI > 5 | SI > 10 |

Nt-INSP-3

| | | | | | | | | |
|---|---|---|---|---|---|---|---|---|
| SLYQLENYVN | 9 | 4 | 2 | 1 | 4 | 4 | 4 | 2 |
| (SEQ ID NO: 1) | (50.0%) | (22.2%) | (11.1%) | (5.6%) | (36.4%) | (36.4%) | (36.4%) | (18.2%) |

oxPTM-INSP-3 derivatives

| | | | | | | | | |
|---|---|---|---|---|---|---|---|---|
| SL-Dopa-QLENY- | 8 | 5 | 3 | 1 | 4 | 3 | 3 | 2 |
| Cysteate-N | (44.4%) | (27.8%) | (16.7%) | (5.6%) | (36.4%) | (27.3%) | (27.3%) | (18.2%) |
| (SEQ ID NO: 2) | | | | | | | | |
| SL-Dopa-QLENDopa- | 6 | 3 | 1 | 1 | 2 | 1 | 0 | 0 |
| Cysteate-N | (33.3%) | (16.7%) | (5.5%) | (5.6%) | (18.2%) | (9.10%) | (0.0%) | (0.0%) |
| (SEQ ID NO: 3) | | | | | | | | |
| at least one oxPTM- | 10 | 6 | 2 | 2 | 5 | 3 | 3 | 2 |
| INSP-3 derivative | (55.6%) | (33.3%) | (10.5%) | (10.5%) | (45.5%) | (27.3%) | (27.3%) | (18.2%) |

Nt-INSP-4

| | | | | | | | | |
|---|---|---|---|---|---|---|---|---|
| LVEALYLVCGERGFFYTPKT | 13 | 10 | 7 | 6 | 7 | 5 | 3 | 2 |
| (SEQ ID NO: 4) | (72.2%) | (55.6%) | (38.9%) | (33.3%) | (63.6%) | (45.5%) | (27.3%) | (18.2%) |

Nt-INSP-6

| | | | | | | | | |
|---|---|---|---|---|---|---|---|---|
| ERGFFYTPKTR | 8 | 3 | 3 | 3 | 3 | 0 | 0 | 0 |
| (SEQ ID NO: 8) | (44.4%) | (16.7%) | (16.7%) | (16.7%) | (27.3%) | (0.0%) | (0.0%) | (0.0%) |

oxPTM-INS-6 derivatives

| | | | | | | | | |
|---|---|---|---|---|---|---|---|---|
| ERGYY-Dopa-TPKT | 5 | 0 | 0 | 0 | 2 | 1 | 0 | 0 |
| (SEQ ID NO: 7) | (27.8%) | (0.0%) | (0.0%) | (0.0%) | (18.2%) | (9.10%) | (0.0%) | (0.0%) |
| ERGYYYTPKT | 4 | 2 | 2 | 1 | 3 | 2 | 2 | 1 |
| (SEQ ID NO: 6) | (22.2%) | (11.1%) | (11.1%) | (5.6%) | (27.3%) | (18.2%) | (18.2%) | (9.1%) |
| ERGYYYTPKTR | 10 | 3 | 3 | 3 | 2 | 1 | 1 | 1 |
| (SEQ ID NO: 9) | (55.6%) | (16.7%) | (16.7%) | (16.7%) | (18.2%) | (9.10%) | (9.1%) | (9.1%) |
| at least one oxPTM- | 13 | 3 | 3 | 3 | 3 | 2 | 2 | 0 |
| INS-6 derivative | (72.2%)* | (16.7%) | (16.7%) | (16.7%) | (27.3%) | (18.2%) | (18.2%) | (0.0%) |

*p vs. controls = 0.02

### REFERENCES

[1] Atkinson M A, Eisenbarth G S, Michels A W (2014) Type 1 diabetes. Lancet 383(9911): 69-82. 10.1016/S0140-6736(13)60591-7
[2] Bluestone J A, Herold K, Eisenbarth G (2010) Genetics, pathogenesis and clinical interventions in type 1 diabetes. Nature 464(7293): 1293-1300. 10.1038/nature08933
[3] Bonifacio E (2015) Predicting type 1 diabetes using biomarkers. Diabetes Care 38(6): 989-996. 10.2337/dc15-0101
[4] Ziegler A G, Rewers M, Simell O, et al. (2013) Seroconversion to multiple islet autoantibodies and risk of progression to diabetes in children. JAMA 309(23): 2473-2479. 10.1001/jama.2013.6285 1697963 [pii]
[5] Babon J A, DeNicola M E, Blodgett D M, et al. (2016) Analysis of self-antigen specificity of islet-infiltrating T cells from human donors with type 1 diabetes. Nat Med 22(12): 1482-1487. 10.1038/nm.4203
[6] Coppieters K T, Dotta F, Amirian N, et al. (2012) Demonstration of islet-autoreactive CD8 T cells in insulitic lesions from recent onset and long-term type 1 diabetes patients. J Exp Med 209(1): 51-60. 10.1084/jem.20111187
[7] Pietropaolo M, Towns R, Eisenbarth G S (2012) Humoral autoimmunity in type 1 diabetes: prediction, significance, and detection of distinct disease subtypes. Cold Spring Harb Perspect Med 2(10). 10.1101/cshperspect.a012831
[8] Michels A W, Landry L G, McDaniel K A, et al. (2017) Islet-Derived CD4 T Cells Targeting Proinsulin in Human Autoimmune Diabetes. Diabetes 66(3): 722-734. 10.2337/db16-1025
[9] James E A, Mallone R, Kent S C, DiLorenzo T P (2020) T-Cell Epitopes and Neo-epitopes in Type 1 Diabetes: A Comprehensive Update and Reappraisal. Diabetes 69(7): 1311-1335. 10.2337/dbi19-0022
[10] Laine A P, Holmberg H, Nilsson A, et al. (2007) Two insulin gene single nucleotide polymorphisms associated with type 1 diabetes risk in the Finnish and Swedish populations. Dis Markers 23(3): 139-145. 10.1155/2007/574363
[11] McGinty J W, Chow I T, Greenbaum C, Odegard J, Kwok W W, James E A (2014) Recognition of posttranslationally modified GAD65 epitopes in subjects with type 1 diabetes. Diabetes 63(9): 3033-3040.10.2337/db13-1952 db13-1952 [pii]
[12] Rondas D, Crevecoeur I, D'Hertog W, et al. (2015) Citrullinated glucose-regulated protein 78 is an autoantigen in type 1 diabetes. Diabetes 64(2): 573-586. 10.2337/db14-0621 db14-0621 [pii]
[13] Arribas-Layton D, Guyer P, Delong T, et al. (2020) Hybrid Insulin Peptides Are Recognized by Human T Cells in the Context of DRB1*04:01. Diabetes 69(7): 1492-1502. 10.2337/db19-0620
[14] Delong T, Wiles T A, Baker R L, et al. (2016) Pathogenic CD4 T cells in type 1 diabetes recognize epitopes formed by peptide fusion. Science 351(6274): 711-714. 10.1126/science.aad2791 351/6274/711 [pii]
[15] Yang C, Kelaini S, Caines R, Margariti A (2018) RBPs Play Important Roles in Vascular Endothelial Dysfunction Under Diabetic Conditions. Front Physiol 9: 1310. 10.3389/fphys.2018.01310
[16] Mannering S I, Harrison L C, Williamson N A, et al. (2005) The insulin A-chain epitope recognized by human T cells is posttranslationally modified. J Exp Med 202(9): 1191-1197. 10.1084/jem.20051251
[17] Acevedo-Calado M, James E A, Morran M P, et al. (2017) Identification of Unique Antigenic Determinants in the Amino Terminus of IA-2 (ICA512) in Childhood and Adult Autoimmune Diabetes: New Biomarker Development. Diabetes Care 40(4): 561-568. 10.2337/dc16-1527
[18] Strollo R, Vinci C, Arshad M H, et al. (2015) Antibodies to post-translationally modified insulin in type 1 diabetes. Diabetologia 58(12): 2851-2860. 10.1007/s00125-015-3746-x
[19] Strollo R, Vinci C, Napoli N, Pozzilli P, Ludvigsson J, Nissim A (2017) Antibodies to post-translationally modified insulin as a novel biomarker for prediction of type 1 diabetes in children. Diabetologia 60(8): 1467-1474. 10.1007/s00125-017-4296-1
[20] Micsonai A, Wien F, Kernya L, et al. (2015) Accurate secondary structure prediction and fold recognition for circular dichroism spectroscopy. Proc Natl Acad Sci USA 112(24): E3095-3103. 10.1073/pnas.1500851112
[21] DeLong E R, DeLong D M, Clarke-Pearson D L (1988) Comparing the areas under two or more correlated receiver operating characteristic curves: a nonparametric approach. Biometrics 44(3): 837-845
[22] Torosantucci R, Mozziconacci O, Sharov V, Schoneich C, Jiskoot W (2012) Chemical modifications in aggregates of recombinant human insulin induced by metal-catalyzed oxidation: covalent cross-linking via michael addition to tyrosine oxidation products. Pharm Res 29(8): 2276-2293. 10.1007/s11095-012-0755-z
[23] Guedes S, Vitorino R, Domingues M R, Amado F, Domingues P (2010) Oxidative modifications in glycated insulin. Anal Bioanal Chem 397(5): 1985-1995. 10.1007/s00216-010-3757-x
[24] Ostrov D A, Alkanani A, McDaniel K A, et al. (2018) Methyldopa blocks MHC class II binding to disease-specific antigens in autoimmune diabetes. J Clin Invest 128(5): 1888-1902. 10.1172/JC197739
[25] Aydintug M K, Zhang L, Wang C, et al. (2014) gammadelta T cells recognize the insulin B:9-23 peptide antigen when it is dimerized through thiol oxidation. Mol Immunol 60(2): 116-128. 10.1016/j.molimm.2014.04.007
[26] Hanson C, Lyden E, Furtado J, Van Ormer M, Anderson-Berry A (2016) A Comparison of Nutritional Antioxidant Content in Breast Milk, Donor Milk, and Infant Formulas. Nutrients 8(11). E681
   [pii]
   nu8110681 [pii]
   10.3390/nu8110681
[27] Sharma V, Shahina K, Srivastava M K, Nanda S, Mishra S (2009) Oxidative stress and coxsackievirus infections as mediators of beta cell damage: A review. Sci Res Essays 4(2): 042-058
[28] Cnop M, Welsh N, Jonas J C, Jorns A, Lenzen S, Eizirik D L (2005) Mechanisms of pancreatic beta-cell death in type 1 and type 2 diabetes: many differences, few similarities. Diabetes 54 Suppl 2: S97-107. 54/suppl_2/S97 [pii]
[29] Burg A R, Das S, Padgett L E, Koenig Z E, Tse H M (2018) Superoxide Production by NADPH Oxidase Intensifies Macrophage Antiviral Responses during Diabetogenic Coxsackievirus Infection. J Immunol 200(1): 61-70. 10.4049/jimmunol.1700478
[30] Marra G, Cotroneo P, Pitocco D, et al. (2002) Early increase of oxidative stress and reduced antioxidant defenses in patients with uncomplicated type 1 diabetes: a case for gender difference. Diabetes Care 25(2): 370-375
[31] Matteucci E, Giampietro O (2000) Oxidative stress in families of type 1 diabetic patients. Diabetes Care 23(8): 1182-1186
[32] Liu C W, Bramer L, Webb-Robertson B J, Waugh K, Rewers M J, Zhang Q (2018) Temporal expression profiling of plasma proteins reveals oxidative stress in early stages of Type 1 Diabetes progression. J Proteomics 172: 100-110. 10.1016/j.jprot.2017.10.004
[33] Ellervik C, Mandrup-Poulsen T, Nordestgaard B G, et al. (2001) Prevalence of hereditary haemochromatosis in late-onset type 1 diabetes mellitus: a retrospective study. Lancet 358(9291): 1405-1409. 10.1016/S0140-6736(01)06526-6
[34] Ellervik C, Mandrup-Poulsen T, Andersen H U, et al. (2011) Elevated transferrin saturation and risk of diabetes: three population-based studies. Diabetes Care 34(10): 2256-2258. 10.2337/dc11-0416
[35] Lenzen S (2008) The mechanisms of alloxan- and streptozotocin-induced diabetes. Diabetologia 51(2): 216-226. 10.1007/s00125-007-0886-7
[36] Weide L G, Lacy P E (1991) Low-dose streptozocin-induced autoimmune diabetes in islet transplantation model. Diabetes 40(9): 1157-1162. 10.2337/diab.40.9.1157
[37] Yang J, Wen X, Xu H et al (2017). Antigen-Specific T Cell Analysis Reveals That Active Immune Responses to p Cell Antigens Are Focused on a Unique Set of Epitopes. J Immunol 199(1):91-96. 10.4049/jimmunol.1601570

1. A peptide comprising an amino acid sequence selected from the group comprising:
(i)
(SEQ ID NO: 1)
SLYQLENYCN
or an oxidised version thereof;
(ii)
(SEQ ID NO: 2)
SL-dihydroxyphenylalanine-QLENY-Cysteate-N;
(iii)
(SEQ ID NO: 3)
SL-dihydroxyphenylalanine-QLEN-
dihydroxyphenylalanine-cysteate-N;
(iv)
(SEQ ID NO: 4)
LVEALYLVCGERGFFYTPKT
or an oxidised version thereof;
(v)
(SEQ ID NO: 5)
ERGFFYTPKT
or an oxidised version thereof;
(vi)
(SEQ ID NO: 6)
ERGYYYTPKT;
(vii)
(SEQ ID NO: 7)
ERGYY-dihydroxyphenylalanine-TPKT;
(viii)
(SEQ ID NO: 8)
ERGFFYTPKTR
or an oxidised version thereof;
(ix)
(SEQ ID NO: 9)
ERGYYYTPKTR;
(x)
(SEQ ID NO: 10)
YLVCGERGFF
or an oxidised version thereof;
(xi)
(SEQ ID NO: 11)
LVEALYLVCGER
or an oxidised version thereof; and
(xii)
(SEQ ID NO: 12)
FVNQHLC
or an oxidised version thereof.

2. A method of diagnosing type 1 diabetes (T1D) in a subject comprising:
testing a sample from the subject for the presence or absence of antibodies against one or more peptides as defined in claim 1;
wherein the presence of antibodies against one or more peptides as defined in claim 1 in the sample is indicative of T1D in the subject.

3. A method according to claim 2, wherein the sample is tested for the presence or absence of antibodies against at least 2, at least 3, or 4 of the peptides as defined in claim 1.

4. A method according to any one of claims 1 to 3, wherein the sample is blood or serum or plasma.

5. A method according to any one of claims 1 to 4, wherein the presence or absence of antibodies against one or more peptides as defined in claim 1 is determined using an ELISA assay.

6. A method according to any one of claims 1 to 5, wherein the subject has not been diagnosed with diabetes.

7. A method according to any one of claims 1 to 6, wherein the subject has no insulin autoantibodies (IAA), ICA, GADA, IA2A and/or ZnT8A.

8. A method of treating type 1 diabetes (T1D) in a subject in need thereof, comprising:
testing a sample from the subject for the presence or absence of antibodies against one or more peptides as defined in claim 1;
identifying the presence of antibodies against one or more peptides as defined in claim 1; and
administering a therapeutic agent for T1D to the subject.

9. The method of claim 8, wherein the therapeutic agent is insulin, an immunotherapeutic administered as vaccine or an immunosuppressive drug.

10. A method of diagnosing latent autoimmune diabetes in adults (LADA) in a subject comprising:
testing a sample from the subject for the presence or absence of antibodies against one or more peptides as defined in claim 1;
wherein the presence of antibodies against one or more peptides as defined in claim 1 in the sample is indicative of LADA in the subject.

11. A method according to claim 10, wherein the subject has been diagnosed with type 2 diabetes.

12. A method according to claim 10 or 11, wherein the sample is blood or serum or plasma.

13. A method according to any one of claims 10 to 12, wherein the subject has no IAA, IA2A, GADA, ICA and/or ZnT8A.

14. A method of treating latent autoimmune diabetes in adults (LADA) in a subject in need thereof, comprising:
testing a sample from the subject for the presence or absence of antibodies against one or more peptides as defined in claim 1;
identifying the presence of antibodies against one or more peptides as defined in claim 1 in the sample; and
administering a therapeutic agent for LADA to the subject.

15. The method of claim 14, wherein the therapeutic agent is insulin, an immunotherapeutic administered as vaccine or an immunosuppressive drug.

16. A method of determining the therapeutic effectiveness of a therapeutic agent in treating T1D or LADA in a subject diagnosed with T1D or LADA comprising:
i) determining the level of antibodies against one or more peptides as defined in claim 1 in a first sample that has been obtained from a subject prior to administering the therapeutic agent to the subject;
ii) determining the level of antibodies against one or more peptides as defined in claim 1 in a second sample that has been obtained from the subject after administering the therapeutic agent to the subject; and
iii) comparing the levels of antibodies against one or more peptides as defined in claim 1 between the first and second samples;
wherein a decrease in the level of antibodies against one or more peptides as defined in claim 1 in the second sample when compared to the first sample is indicative of therapeutic effectiveness of the therapeutic agent.

17. A method of determining the therapeutic effectiveness of a therapeutic agent in treating T1D or LADA in a subject diagnosed with T1D or LADA comprising:
i) obtaining a first sample from the subject;
ii) determining the level of antibodies against one or more peptides as defined in claim 1 in the first sample;
iii) administering the therapeutic agent to the subject;
iv) obtaining a second sample from the subject;
v) determining the level of antibodies against one or more peptides as defined in claim 1 in the second sample; and
vi) comparing the levels of antibodies against one or more peptides as defined in claim 1 between the first and second samples;
wherein a decrease in the level of antibodies against one or more peptides as defined in claim 1 in the second sample when compared to the first sample is indicative of therapeutic effectiveness of the therapeutic agent.

18. The method of claim 17, further comprising the step of administering a therapeutic agent for T1D or LADA to the subject.

19. The method of claim 18, wherein the therapeutic agent is insulin, an immunotherapeutic administered as vaccine or an immunosuppressive drug.

20. A kit for diagnosing T1D or LADA in a subject comprising at least two peptides as defined in claim 1.

21. A method of diagnosing type 1 diabetes (T1D) in a subject comprising:
testing a sample from the subject for the presence or absence of T cells that are specific for one or more peptides as defined in claim 1;
wherein the presence of T cells that are specific for one or more peptides as defined in claim 1 in the sample is indicative of T1D in the subject.

22. A method of treating type 1 diabetes (T1D) in a subject in need thereof, comprising:
testing a sample from the subject for the presence or absence of T cells that are specific for one or more peptides as defined in claim 1;
identifying the presence of T cells that are specific for one or more peptides as defined in claim 1; and
administering a therapeutic agent for T1D to the subject.

23. A method of diagnosing latent autoimmune diabetes in adults (LADA) in a subject comprising:
testing a sample from the subject for the presence or absence of T cells that are specific for one or more peptides as defined in claim 1;
wherein the presence of T cells that are specific for one or more peptides as defined in claim 1 in the sample is indicative of LADA in the subject.

24. A method of treating latent autoimmune diabetes in adults (LADA) in a subject in need thereof, comprising:
testing a sample from the subject for the presence or absence of T cells that are specific for one or more peptides as defined in claim 1;
identifying the presence of T cells that are specific for one or more peptides as defined in claim 1 in the sample; and
administering a therapeutic agent for LADA to the subject.

25. A method of determining the therapeutic effectiveness of a therapeutic agent in treating T1D or LADA in a subject diagnosed with T1D or LADA comprising:
i) determining the level of T cells that are specific for one or more peptides as defined in claim 1 in a first sample that has been obtained from a subject prior to administering the therapeutic agent to the subject;
ii) determining the level of T cells that are specific for one or more peptides as defined in claim 1 in a second sample that has been obtained from the subject after administering the therapeutic agent to the subject; and
iii) comparing the levels of T cells that are specific for one or more peptides as defined in claim 1 between the first and second samples;
wherein a decrease in the level of T cells that are specific for one or more peptides as defined in claim 1 in the second sample when compared to the first sample is indicative of therapeutic effectiveness of the therapeutic agent.

26. A method of determining the therapeutic effectiveness of a therapeutic agent in treating T1D or LADA in a subject diagnosed with T1D or LADA comprising:
i) obtaining a first sample from the subject;
ii) determining the level of T cells that are specific for one or more peptides as defined in claim 1 in the first sample;
iii) administering the therapeutic agent to the subject;
iv) obtaining a second sample from the subject;
v) determining the level of T cells that are specific for one or more peptides as defined in claim 1 in the second sample; and
vi) comparing the levels of T cells that are specific for one or more peptides as defined in claim 1 between the first and second samples;
wherein a decrease in the level of T cells that are specific for one or more peptides as defined in claim 1 in the second sample when compared to the first sample is indicative of therapeutic effectiveness of the therapeutic agent.
